SEQUENCE LISTING (53 sequences)

SEQ ID NO: 1 - DNA, 81 bp, Homo sapiens
  source 1..81: mol_type = genomic DNA
ttgaaaatccgggggagagggtgtaaatctcgcgccgggccgtacccatatccgcagcaggtctccaaggtgaacagcctc

SEQ ID NO: 2 - DNA, 61 bp, Homo sapiens
  source 1..61: mol_type = genomic DNA
acttcgaacgcacttgcggccccgggttcctcccggggctacgcctgtctgagcgtcgctt

SEQ ID NO: 3 - DNA, 5066 bp, Homo sapiens
  source 1..5066: mol_type = genomic DNA
cgcgacctcagatcagacgtggcgacccgctgaatttaagcatattagtcagcggaggagaagaaactaaccaggattccctcagtaacggcgagtgaacagggaagagcccagcgccgaatccccgccccgcggcggggcgcgggacatgtggcgtacggaagacccgctccccggcgccgctcgtggggggcccaagtccttctgatcgaggcccagcccgtggacggtgtgaggccggtagcggcccccggcgcgccgggcccgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgggtccggccgtgtcggcggcccggcggatctttcccgccccccgttcctcccgacccctccacccgccctcccttcccccgccgcccctcctcctcctccccggagggggcgggctccggcgggtgcgggggtgggcgggcggggccgggggtggggtcggcgggggaccgtcccccgaccggcgaccggccgccgccgggcgcatttccaccgcggcggtgcgccgcgaccggctccgggacggctgggaaggcccggcggggaaggtggctcggggggccccgtccgtccgtccgtccgtcctcctcctcccccgtctccgccccccggccccgcgtcctccctcgggagggcgcgcgggtcggggcggcggcggcggcggcggtggcggcggcggcggcggcggcgggaccgaaaccccccccgagtgttacagcccccccggcagcagcactcgccgaatcccggggccgagggagcgagacccgtcgccgcgctctcccccctcccggcgcccacccccgcggggaatcccccgcgaggggggtctcccccgcgggggcgcgccggcgtctcctcgtgggggggccgggccacccctcccacggcgcgaccgctctcccacccctcctccccgcgcccccgccccggcgacggggggggtgccgcgcgcgggtcggggggcggggcggactgtccccagtgcgccccgggcgggtcgcgccgtcgggcccgggggaggttctctcggggccacgcgcgcgtcccccgaagagggggacggcggagcgagcgcacggggtcggcggcgacgtcggctacccacccgacccgtcttgaaacacggaccaaggagtctaacacgtgcgcgagtcgggggctcgcacgaaagccgccgtggcgcaatgaaggtgaaggccggcgcgctcgccggccgaggtgggatcccgaggcctctccagtccgccgagggcgcaccaccggcccgtctcgcccgccgcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcagacccgacgcacccccgccacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcgagagtggacgggagcggcgggggcggcgcgcgcgcgcgcgcgtgtggtgtgcgtcggagggcggcggcggcggcggcggcgggggtgtggggtccttcccccgcccccccccccacgcctcctcccctcctcccgcccacgccccgctccccgcccccggagccccgcggacgctacgccgcgacgagtaggagggccgctgcggtgagccttgaagcctagggcgcgggcccgggtggagccgccgcaggtgcagatcttggtggtagtagcaaatattcaaacgagaactttgaaggccgaagtggagaagggttccatgtgaacagcagttgaacatgggtcagtcggtcctgagagatgggcgagcgccgttccgaagggacgggcgatggcctccgttgccctcggccgatcgaaagggagtcgggttcagatccccgaatccggagtggcggagatgggcgccgcgaggcgtccagtgcggtaacgcgaccgatcccggagaagccggcgggagccccggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccgcgcgcgccgctcgctccctccccgccccgcgccctctctctctctctctcccccgctccccgtcctcccccctccccgggggagcgccgcgtgggggcggcggcggggggagaagggtcggggcggcaggggccggcggcggcccgccgcggggccccggcggcgggggcacggtcccccgcgaggggggcccgggcacccggggggccggcggcggcggcgactctggacgcgagccgggcccttcccgtggatcgccccagctgcggcgggcgtcgcggccgcccccggggagcccggcgggcgccggcgcgcccccccccccaccccacgtctcgtcgcgcgcgcgtccgctgggggcggggagcggtcgggcggcggcggtcggcgggcggcggggcggggcggttcgtccccccgccctacccccccggccccgtccgccccccgttcccccctcctcctcggcgcgcggcggcggcggcgggcggcggaggggccgcgggccggtcccccccgccgggtccgcccccggggccgcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctgcgggccgccggtgaaataccactactctgatcgttttttcactgacccggtgaggcgggggggcgagccccgaggggctctcgcttctggcgccaagcgcccggccgcgcgccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcggaacgatacggcagcgccgcggagcctcggttggcctcggatagccggtcccccgcctgtccccgccggcgggccgcccccccctccacgcgccccgcgcgcgcgggagggcgcgtgccccgccgcgcgccgggaccggggtccggtgcggagtgcccttcgtcctgggaaacggggcgcggctggaaaggcggccgccccctcgcccgtcacgcaccgcacgttcgtggggaacctggcgctaaaccattcgtagacgacctgcttctgggtcggggtttcgtacgtagcagagcagctccctcgctgcgatctattgaaagtcagccctcgacacaagggtttgtc

SEQ ID NO: 4 - DNA, 1869 bp, Homo sapiens
  source 1..1869: mol_type = genomic DNA
tacctggttgatcctgccagtagcatatgcttgtctcaaagattaagccatgcatgtctgagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 5 - DNA, 157 bp, Homo sapiens
  source 1..157: mol_type = genomic DNA
cgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcgctt

SEQ ID NO: 6 - DNA, 3654 bp, Homo sapiens
  source 1..3654: mol_type = genomic DNA
gctgacacgctgtcctctggcgacctgtcgctggagaggttgggcctccggatgcgcgcggggctctggcctaccggtgacccggctagccggccgcgctcctgcttgagccgcctgccggggcccgcgggcctgctgttctctcgcgcgtccgagcgtcccgactcccggtgccggcccgggtccgggtctctgacccacccgggggcggcggggaaggcggcgagggccaccgtgcccccgtgcgctctccgctgcgggcgcccggggcggccgcgacaaccccaccccgctggctccgtgccgtgcgtgtcaggcgttctcgtctccgcggggttgtccgccgccccttccccggagtggggggttggccggagccgatcggctcgctggccggccggccggcctccgctcccggggggctcttcgtgatcgatgtggtgacgtcgtgctctcccgggccgggtccgagccgcgacgggcgaggggcggacgttcgtggcgaacgggaccgtccttctcgctccgccccgcgggggtcccctcgtctctcctctccccgcccgccggcggtgcgtgtgggaaggcgtggggtgcggaccccggcccgacctcgccgtcccgcccgccgccttctgcgtcgcggggcgggccggcggggtcctctgacgcggcagacagccctcgctgtcgcctccagtggttgtcgacttgcgggcggcccccctccgcggcggtgggggtgccgtcccgccggcccgtcgtgctgccctctcggggggtttgcgcgagcgtcggctccgcctgggcccttgcggtgctcctggagcgctccgggttgtccctcaggtgcccgaggccgaacggtggtgtgtcgttcccgcccccggcgccccctcctccggtcgccgccgcggtgtccgcgcgtgggtcctgagggagctcgtcggtgtggggttcgaggcggtttgagtgagacgagacgagacgcgcccctcccacgcggggaagggcgcccgcctgctctcggtgagcgcacgtcccgtgctcccctctggcgggtgcgcgcgggccgtgtgagcgatcgcggtgggttcgggccggtgtgacgcgtgcgccggccggccgccgaggggctgccgttctgcctccgaccggtcgtgtgtgggttgacttcggaggcgctctgcctcggaaggaaggaggtgggtggacgggggggcctggtggggttgcgcgcacgcgcgcaccggccgggcccccgccctgaacgcgaacgctcgaggtggccgcgcgcaggtgtttcctcgtaccgcagggccccctcccttccccaggcgtccctcggcgcctctgcgggcccgaggaggagcggctggcgggtggggggagtgtgacccaccctcggtgagaaaagccttctctagcgatctgagaggcgtgccttgggggtaccggatcccccgggccgccgcctctgtctctgcctccgttatggtagcgctgccgtagcgacccgctcgcagaggaccctcctccgcttccccctcgacggggttgggggggagaagcgagggttccgccggccaccgcggtggtggccgagtgcggctcgtcgcctactgtggcccgcgcctcccccttccgagtcgggggaggatcccgccgggccgggcccggcgttcccagcgggttgggacgcggcggccggcgggcggtgggtgtgcgcgcccggcgctctgtccggcgcgtgaccccctccgccgcgagtcggctctccgcccgctcccgtgccgagtcgtgaccggtgccgacgaccgcgtttgcgtggcacggggtcgggcccgcctggccctgggaaagcgtcccacggtgggggcgcgccggtctcccggagcgggaccgggtcggaggatggacgagaatcacgagcgacggtggtgcgggcgtgtcgggttcgtggctgcggtcgctccggggcccccggtggcggggccccggggctcgcgaggcggttctcggtgggggccgagggccgtccggcgtcccaggcggggcgccgcgggaccgccctcgtgtctgtggcggtgggatcccgcggccgtgttttcctggtggcccggccgtgcctgaggtttctccccgagccgccgcctctgcgggctcccgggtgcccttgccctcgcggtccccggccctcgcccgtctgtgccctcttccccgcccgccgcccgccgatcctcttcttccccccgagcggctcaccggcttcacgtccgttggtggccccgcctgggaccgaacccggcaccgcctcgtggggcgccgccgccggccactgatcggcccggcgtccgcgtcccccggcgcgcgccttggggaccgggtcggtggcgccccgcgtggggcccggtgggcttcccggagggttccgggggtcggcctgcggcgcgtgcgggggaggagacggttccgggggaccggccgcgactgcggcggcggtggtgggggcagccgcggggatcgccgagggccggtcggccgccccgggtgccgcgcggtgccgccggcggcggtgaggccccgcgcgtgtgtcccggccgcggtcggccgcgctcgaggggtccccgtggcgtccccttccccgccggccgcctttctcgcgccttccccgtcgccccggcctcgcccgtggtctctcgtcttctcccggcccgctcttccgaaccgggtcggcgcgtcccccgggtgcgcctcgcttcccgggcctgccgcggcccttccccgaggcgtccgtcccgggcgtcggcgtcggggagagcccgtcctccccgcgtggcgtcgccccgttcggcgcgcgcgtgcgcccgagcgcggcccggtggtccctgccggacaggcgttcgtgcgacgtgtggcgtgggtcgacctccgccttgccggtcgctcgccctttccccgggtcggggggtggggcccgggccggggcctcggccccggtcgcggtcccccgtcccgggcgggggcgggcgcgccggccggcctcggtcggccctcccttggccgtcgtgtggcgtgtgccacccctgcgcccgcgcccgccggcggggctcggagccgggcttcggccgggccccgggccctcgaccggaccggtgcgcgggcgctgcggccgcacggcgcgactgtccccgggccgggcaccgcggtccgcctctcgctcgccgcccggacgtcggggccgccccgcggggcgggcggagcgccgtccccgcctcgccgccgcccgcgggcgccggccgcgcgcgcgcgcgcgtggccgccggtccctcccggccgccgggcgcgggtcgggccgtccgcctcctcgcgggcgggcgcgacgaagaagcgtcgcgggtctgtggcgcggggccccggtggtcgtgtcgcgtggggggcgggtggttggggcgtccggttcgccgcgccccgccccggccccaccggtcccggccgccgcccccgcgcccgctcgctccctcccgtccgcccgtccgcggcccgtccgtccgtccgtcgtcctcctcgcttgcggggcgccgggcccgtcctcgcgaggccccccggccggccgtccggccgcgtcggggcctcgccgcgctctaccttacc

SEQ ID NO: 7 - DNA, 1077 bp, Homo sapiens
  source 1..1077: mol_type = genomic DNA
acggagcccggagggcgaggcccgcggcggcgccgccgccgccgcgcgcttccctccgcacacccacccccccaccgcgacgcggcgcgtgcgcgggcggggcccgcgtgcccgttcgttcgctcgctcgttcgttcgccgcccggccccgccggccgcgagagccggagaactcgggagggagacgggggagagagagagagagagagaaagagaaagaagggcgtgtcgttggtgtgcgcgtgtcgtggggccggcgggcggcggggagcggtccccggccgcggccccgacgacgtgggtgtcggcgggcgcgggggcggttctcggcggcgtcgcggcgggtctgggggggtctcggtgccctcctccccgccggggcccgtcgtccggccccgccgcgccggctccccgtcttcggggccggccggattcccgtcgcctccgccgcgccgctccgcgccgccgggcacggccccgctcgctctccccggccttcccgctagggcgtctcgagggtcgggggccggacgccggtcccctcccccgcctcctcgtccgcccccccgccgtccaggtacctagcgcgttccggcgcggaggtttaaagaccccttggggggatcgcccgtccgcccgtgggtcgggggcggtggtgggcccgcgggggagtcccgtcgggaggggcccggcccctcccgcgcctccaccgcggactccgctccccggccggggccgcgccgccgccgccgccgcggcggccgtcgggtgggggctttacccggcggccgtcgcgcgcctgccgcgcgtgtggcgtgcgccccgcgccgtgggggcgggaacccccgggcgcctgtggggtggtgtccgcgctcgcccccgcgtgggcggcgcgcgcctccccgtggtgtgaaaccttccgacccctctccggagtccggtcccgtttgctgtctcgtctggccggcctgaggcaaccccctctcctcttgggcgggggggggggggacgtgccgcgccaggaagggcctcctcccggtgcgtcgtcgggagcgccctcgccaaatcgacctcgta

SEQ ID NO: 8 - RNA, 20 nt, synthetic construct
  source 1..20: mol_type = other RNA
  misc_difference 1: note = May be a 2'-O-alkyl, 2'-deoxy-2'-fluoro, 2'-deoxy, 2'-H (deoxyribonucleotides), or 2'-O-methyl modified nucleotide
  misc_difference 2: note = May be a 2'-O-alkyl, 2'-deoxy-2'-fluoro, 2'-deoxy, 2'-H (deoxyribonucleotides), or 2'-O-methyl modified nucleotide
  misc_difference 3: note = May be a 2'-O-alkyl, 2'-deoxy-2'-fluoro, 2'-deoxy, 2'-H (deoxyribonucleotides), or 2'-O-methyl modified nucleotide
  misc_difference 4: note = May be a 2'-O-alkyl, 2'-deoxy-2'-fluoro, 2'-deoxy, 2'-H (deoxyribonucleotides), or 2'-O-methyl modified nucleotide
  misc_difference 5: note = May be a 2'-O-alkyl, 2'-deoxy-2'-fluoro, 2'-deoxy, 2'-H (deoxyribonucleotides), or 2'-O-methyl modified nucleotide
  misc_difference 16: note = May be a 2'-O-alkyl, 2'-deoxy-2'-fluoro, 2'-deoxy, 2'-H (deoxyribonucleotides), or 2'-O-methyl modified nucleotide
  misc_difference 17: note = May be a 2'-O-alkyl, 2'-deoxy-2'-fluoro, 2'-deoxy, 2'-H (deoxyribonucleotides), or 2'-O-methyl modified nucleotide
  misc_difference 18: note = May be a 2'-O-alkyl, 2'-deoxy-2'-fluoro, 2'-deoxy, 2'-H (deoxyribonucleotides), or 2'-O-methyl modified nucleotide
  misc_difference 19: note = May be a 2'-O-alkyl, 2'-deoxy-2'-fluoro, 2'-deoxy, 2'-H (deoxyribonucleotides), or 2'-O-methyl modified nucleotide
  misc_difference 20: note = May be a 2'-O-alkyl, 2'-deoxy-2'-fluoro, 2'-deoxy, 2'-H (deoxyribonucleotides), or 2'-O-methyl modified nucleotide
ggtctccaaggtgaacagcc

SEQ ID NO: 9 - protein, 60 aa, synthetic construct
PRPRPRPRPRPRPRPRPRPRPRPRPRPRPRPRPRPRPRPRPRPRPRPRPRPRPRPRPRPR

SEQ ID NO: 10 - protein, 30 aa, synthetic construct
GPGPGPGPGPGPGPGPGPGPGPGPGPGPGP

SEQ ID NO: 11 - protein, 30 aa, synthetic construct
GRGRGRGRGRGRGRGRGRGRGRGRGRGRGR

SEQ ID NO: 12 - protein, 30 aa, synthetic construct
PRPRPRPRPRPRPRPRPRPRPRPRPRPRPR

SEQ ID NO: 13 - protein, 100 aa, synthetic construct
  VARIANT 1..100: note = This sequence may encompass 10, 20, 30, or 50 GR repeats
GRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGRGR

SEQ ID NO: 14 - protein, 100 aa, synthetic construct
  VARIANT 1..100: note = This sequence may encompass 10, 20, 30, or 50 PR repeats
PRPRPRPRPRPRPRPRPRPRPRPRPRPRPRPRPRPRPRPRPRPRPRPRPRPRPRPRPRPRPRPRPRPRPRPRPRPRPRPRPRPRPRPRPRPRPRPRPRPR

SEQ ID NO: 15 - RNA, 20 nt, synthetic construct
  source 1..20: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
ggtctccaaggtgaacagcc

SEQ ID NO: 16 - protein, 40 aa, synthetic construct
PRPRPRPRPRPRPRPRPRPRPRPRPRPRPRPRPRPRPRPR

SEQ ID NO: 17 - protein, 100 aa, synthetic construct; identical to SEQ ID NO: 13

SEQ ID NO: 18 - protein, 100 aa, synthetic construct; identical to SEQ ID NO: 14

SEQ ID NO: 19 - protein, 100 aa, synthetic construct; identical to SEQ ID NO: 13
  VARIANT 1..100: note = This sequence may encompass 10, 15, 20, 30, or 50 GR repeats

SEQ ID NO: 20 - protein, 100 aa, synthetic construct; identical to SEQ ID NO: 14
  VARIANT 1..100: note = This sequence may encompass 10, 15, 20, 30, or 50 PR repeats

SEQ ID NO: 21 - protein, 100 aa, synthetic construct
  VARIANT 1..100: note = This sequence may encompass 10, 15, 20, 30, or 50 GP repeats
GPGPGPGPGPGPGPGPGPGPGPGPGPGPGPGPGPGPGPGPGPGPGPGPGPGPGPGPGPGPGPGPGPGPGPGPGPGPGPGPGPGPGPGPGPGPGPGPGPGP

SEQ ID NO: 22 - protein, 30 aa, synthetic construct
GAGAGAGAGAGAGAGAGAGAGAGAGAGAGA

SEQ ID NO: 23 - protein, 100 aa, synthetic construct
  VARIANT 1..100: note = This sequence may encompass 10, 20, 30, or 50 GA repeats
GAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGA

SEQ ID NO: 24 - protein, 100 aa, synthetic construct; identical to SEQ ID NO: 21
  VARIANT 1..100: note = This sequence may encompass 15, 30, or 50 GP repeats

SEQ ID NO: 25 - protein, 100 aa, synthetic construct; identical to SEQ ID NO: 13
  VARIANT 1..100: note = This sequence may encompass 15, 30, or 50 GR repeats

SEQ ID NO: 26 - protein, 100 aa, synthetic construct; identical to SEQ ID NO: 14
  VARIANT 1..100: note = This sequence may encompass 15, 30, or 50 PR repeats

SEQ ID NO: 27 - DNA, 30 bp, synthetic construct
  source 1..30: mol_type = other DNA
gtgtcgatgatcaatgtgtcctgcaattca

SEQ ID NO: 28 - DNA, 20 bp, synthetic construct
  source 1..20: mol_type = other DNA
cgctccaccaactaagaacg

SEQ ID NO: 29 - DNA, 22 bp, synthetic construct
  source 1..22: mol_type = other DNA
cctggttagtttcttctcctcc

SEQ ID NO: 30 - DNA, 24 bp, synthetic construct
  source 1..24: mol_type = other DNA
ccatattgatgccgaacttagtgc

SEQ ID NO: 31 - DNA, 20 bp, synthetic construct
  source 1..20: mol_type = other DNA
ctgttccaagctccggcaaa

SEQ ID NO: 32 - DNA, 20 bp, synthetic construct
  source 1..20: mol_type = other DNA
aatgggcggaggagagtagt

SEQ ID NO: 33 - DNA, 22 bp, synthetic construct
  source 1..22: mol_type = other DNA
ggaggagaagaaactaaccagg

SEQ ID NO: 34 - DNA, 21 bp, synthetic construct
  source 1..21: mol_type = other DNA
gtcttccgtacgccacatgtc

SEQ ID NO: 35 - DNA, 20 bp, synthetic construct
  source 1..20: mol_type = other DNA
ctcaacacgggaaacctcac

SEQ ID NO: 36 - DNA, 20 bp, synthetic construct
  source 1..20: mol_type = other DNA
tctagcgatctgagaggcgt

SEQ ID NO: 37 - DNA, 20 bp, synthetic construct
  source 1..20: mol_type = other DNA
cagcgctaccataacggagg

SEQ ID NO: 38 - DNA, 20 bp, synthetic construct
  source 1..20: mol_type = other DNA
caaccccctctcctcttggg

SEQ ID NO: 39 - DNA, 19 bp, synthetic construct
  source 1..19: mol_type = other DNA
gaggtcgatttggcgaggg

SEQ ID NO: 40 - DNA, 20 bp, synthetic construct
  source 1..20: mol_type = other DNA
ggctggtccgatggtagtgg

SEQ ID NO: 41 - DNA, 25 bp, synthetic construct
  source 1..25: mol_type = other DNA
aaagccagtcaaatttagcagtggg

SEQ ID NO: 42 - DNA, 21 bp, synthetic construct
  source 1..21: mol_type = other DNA
agttggtccgagtgttgtggg

SEQ ID NO: 43 - DNA, 24 bp, synthetic construct
  source 1..24: mol_type = other DNA
aaaacagcaagctagtcaagcgcg

SEQ ID NO: 44 - DNA, 18 bp, synthetic construct
  source 1..18: mol_type = other DNA
tcccacatggtctagcgg

SEQ ID NO: 45 - DNA, 16 bp, synthetic construct
  source 1..16: mol_type = other DNA
ttcccacaccgggagt

SEQ ID NO: 46 - RNA, 20 nt, synthetic construct
  source 1..20: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
gcactgacgatgcgctagca

SEQ ID NO: 47 - RNA, 20 nt, synthetic construct
  source 1..20: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl, 3' Cy3 fluorophore modified nucleotide
ggtctccaaggtgaacagcc

SEQ ID NO: 48 - DNA, 180 bp, synthetic construct
  source 1..180: mol_type = other DNA
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 49 - protein, 72 aa, synthetic construct
GRGRGRGRGRGRGRGRGRGRGRGRGRGRGRGRGRGRGRGRGRGRGRGRGRGRGRGRGRGRGRGRGRGRGRGR

SEQ ID NO: 50 - protein, 20 aa, synthetic construct
GRGRGRGRGRGRGRGRGRGR

SEQ ID NO: 51 - protein, 40 aa, synthetic construct
GRGRGRGRGRGRGRGRGRGRGRGRGRGRGRGRGRGRGRGR

SEQ ID NO: 52 - protein, 60 aa, synthetic construct
GRGRGRGRGRGRGRGRGRGRGRGRGRGRGRGRGRGRGRGRGRGRGRGRGRGRGRGRGRGR

SEQ ID NO: 53 - protein, 20 aa, synthetic construct
PRPRPRPRPRPRPRPRPRPR

(19) United States
(12) Patent Application Publication
(10) Pub. No.: US 2025/0027084 A1
(43) Pub. Date: Jan. 23, 2025

(54) COMPOSITIONS FOR INHIBITING DIPEPTIDE REPEAT PROTEIN-RIBOSOMAL RNA INTERACTION AND USES THEREOF

(71) Applicants: Northwestern University, Evanston, IL (US); The United States of America as Represented by the Secretary, Department of Health and Human Service, Bethesda, MD (US)

(72) Inventors: Sandra Wolin, Rockville, MD (US); Marco Boccitto, Chevy Chase, MD (US); Juan Alberto Ortega Cano, Barcelona (ES); Evangelos Kiskinis, Chicago, IL (US)

(73) Assignees: Northwestern University, Evanston, IL (US); The United States of America as Represented by the Secretary, Department of Health and Human Service, Bethesda, MD (US)

(21) Appl. No.: 18/714,298

(22) PCT Filed: Nov. 29, 2022
(86) PCT No.: PCT/US2022/051249

§ 371 Date: May 29, 2024

Related U.S. Application Data
(60) Provisional application No. 63/284,485, filed on Nov. 30, 2021.

(51) Int. Cl. C12N 15/113

(52) U.S. Cl. CPC C12N 15/113; C12N 2310/321

(57) ABSTRACT

The present disclosure relates generally to compositions and methods for inhibiting dipeptide repeat protein (DPR)-ribosomal RNA (rRNA) interaction. In particular, the present technology relates to administering a therapeutically effective amount of one or more compositions that inhibit DPR-rRNA interaction to a subject diagnosed with, or at risk for DPR-associated pathologies, e.g., amyotrophic lateral sclerosis or frontotemporal dementia.

### CROSS-REFERENCE TO RELATED APPLICATION

This application is the U.S. National Stage of PCT/US2022/051249, which claims the benefit of the priority date of U.S. provisional application, 63/284,485, filed Nov. 30, 2021, the contents of which are incorporated herein by reference in their entirety.

### STATEMENT REGARDING FEDERALLY SPONSORED RESEARCH OR DEVELOPMENT

This invention was made with government support under grant number NS104219 awarded by National Institutes of Health (NIH). The government has certain rights in the invention.

The instant application contains a Sequence Listing which has been submitted in XML format and is hereby incorporated by reference in its entirety. Said XML copy, created on May 24, 2024, is named 121384_0190_SL.xml and is 86,389 bytes.

### TECHNICAL FIELD

The present disclosure relates generally to compositions and methods for inhibiting arginine-rich dipeptide repeat protein (R-DPR)-ribosomal RNA (rRNA) interaction. In particular, the present technology relates to administering a therapeutically effective amount of one or more compositions that inhibit R-DPR-rRNA interaction to a subject diagnosed with, or at risk for R-DPR-associated pathologies, e.g., amyotrophic lateral sclerosis or frontotemporal dementia.

### SUMMARY OF THE PRESENT TECHNOLOGY

In one aspect, the present disclosure provides a single-stranded RNA (ssRNA) construct comprising a sequence that is identical to the sequence of a fragment of a ribosomal RNA (rRNA) subunit selected from the group consisting of the 28S, 18S, and 5.8S subunits, or a fragment of the 5′ETS or ITS1 region of the 47S rRNA precursor, wherein the ssRNA construct comprises one or more modified nucleotides.

In some embodiments, the ssRNA construct comprises a sequence that is identical to the sequence of a fragment of the rRNA 28S subunit. In some embodiments, the fragment of the rRNA 28S subunit comprises all or part of the sequence of SEQ ID NO: 1. In some embodiments, the fragment of the rRNA 28S subunit includes nucleotides 2740 and 2820 of SEQ ID NO: 3.

In some embodiments, the ssRNA construct comprises a sequence that is identical to the sequence of a fragment of the rRNA 5.8S subunit. In some embodiments, the fragment of the rRNA 5.8S subunit comprises all or part of the sequence SEQ ID NO: 2.

In some embodiments, the fragment consists of up to 30 nucleotides.

In some embodiments, the modified nucleotides comprise one or more 2′-modified ribose sugars. In some embodiments, the modified nucleotides are 2′-O-alkyl nucleotides, 2′-deoxy-2′-fluoro nucleotides, 2′-deoxy nucleotides, 2′-H (deoxyribonucleotides), or a combination thereof. In some embodiments, the modified nucleotides are 2′-O-methyl nucleotides.

In some embodiments, the modified nucleotides are at the 5′ end and/or the 3′ end.

In some embodiments, the ssRNA construct comprises a structure of mGmGmUmCmUrCrCrArArGrGrUrGrArAmCmAmGmCmC (SEQ ID NO: 8).

In some embodiments, the ssRNA construct further comprises a covalently linked fluorophore.

In one aspect, the present disclosure provides a composition comprising any of the above ssRNA constructs, and a pharmaceutically acceptable carrier.

In another aspect, the present disclosure provides a method for treating or preventing amyotrophic lateral sclerosis or frontotemporal dementia in a subject thereof, comprising administering to the subject a therapeutically effective amount of an ssRNA construct that inhibits dipeptide repeat (DPR) protein-ribosomal RNA (rRNA) interaction, wherein the ssRNA construct comprises a sequence that is identical to the sequence of a fragment of a ribosomal RNA (rRNA) or a rRNA precursor.

In some embodiments, the subject harbors a heterozygous intronic hexanucleotide (GGGGCC)ₙ repeat expansion in C9ORF72 (C9-HRE) gene. In some embodiments, the subject has an increased expression of one or more of poly-glycine-proline (polyGP), poly-glycine-alanine (polyGA), poly-glycine-arginine (polyGR), poly-proline-arginine (polyPR), and poly-proline-alanine (polyPA) as compared to that observed in a healthy subject. In some embodiments, the the subject has an increased expression of poly-GR and/or poly-PR.

In some embodiments, the subject exhibits one or more signs of impaired ribosomal homeostasis and function selected from the group consisting of a reduced total rRNA level, an increased nucleocytoplasmic (N/C) ratio of rRNA, or a decreased level of de novo protein translation as compared to that observed in a healthy subject.

In some embodiments, the the ssRNA comprises one or more modified nucleotides. In some embodiments, the modified nucleotides comprise 2′-modified ribose sugars. In some embodiments, the modified nucleotides are 2′-O-alkyl nucleotides, 2′-deoxy-2′-fluoro nucleotides, 2′-deoxy nucleotides, 2′-H (deoxyribonucleotides), or a combination thereof. In some embodiments, the modified nucleotides are 2′-O-methyl nucleotides.

### BRIEF DESCRIPTION OF THE DRAWINGS

FIGS. 1A-1H and FIGS. 2A-2J show computational and empirical characterization of the physicochemical features of C9 R-DPRs. FIG. 1A. Drawing of (GP)₁₅ (SEQ ID NO: 10), (GR)₁₅ (SEQ ID NO: 11) and (PR)₁₅ (SEQ ID NO: 12) chemical structures. FIG. 1B. All-atoms molecular dynamics simulation of the secondary structures of (GP)₁₅ (SEQ ID NO: 10), (GR)₁₅ (SEQ ID NO: 11) and (PR)₁₅ (SEQ ID NO: 12). FIG. 1C. Ramachandran plots mapping the frequencies of secondary structures (gray dots) present within (GP)₁₅ (SEQ ID NO: 10), (GR)₁₅ (SEQ ID NO: 11) and (PR)₁₅ (SEQ ID NO: 12). Green and blue areas identify different types of dihedral angles associated to β-sheets, left- and right-handed α-helices as shown in left panel. FIG. 1D. Line graph showing the secondary structure traces of 1 mM (GP)₁₅ (SEQ ID NO: 10), (GR)₁₅ (SEQ ID NO: 11) and (PR)₁₅ (SEQ ID NO: 12) in 10 mM HEPES, 10 mM NaCl pH7.2, analyzed by circular dichroism (CD). FIG. 1E. Line graph showing the secondary structure traces of (GP)₁₅ (SEQ ID NO: 10), (GR)₁₅ (SEQ ID NO: 11) and (PR)₁₅ (SEQ ID NO: 12) analyzed by liquid FTIR. HEPES solution was used as a control. FIG. 1F. All-atoms molecular dynamics simulations of (GR)ₙ(SEQ ID NO: 13) (top) and (PR)ₙ(SEQ ID NO: 14) (bottom) with different number of repeats (n=10, 20, 30, 50) representing their secondary structures. FIG. 1F discloses SEQ ID NOS 50-52, 17, 53, 16, 9, and 18, respectively, in order of appearance. FIG. 1G. Top: schematic representation of the peptide length calculation for (GR)₁₅ (SEQ ID NO: 11) and (PR)₁₅ (SEQ ID NO: 12), measured by the radius of the circle that circumscribes each R-DPR; Bottom: bar graph depicting the calculated radius of gyration (Rg) in (GR)ₙ(SEQ ID NO: 13) and (PR)ₙ(SEQ ID NO: 14) with different number of repeats (n=10, 20, 30, 50). Values are presented as the mean±standard deviation (SD). FIG. 1H. Bar plot showing the number of R-R and H-bond (within the backbone or between backbone and R residues) interactions in (GR)₅₀ (SEQ ID NO: 17) and (PR)₅₀ (SEQ ID NO: 18). Values are presented as the mean±SD. FIG. 2A. Line graphs showing changes in the distance between atoms calculated by root-mean-square deviation (RMSD) over time in (GR)ₙ(SEQ ID NO: 19) (top), (PR)ₙ(SEQ ID NO: 20) (middle), and (GP)ₙ (SEQ ID NO: 21) (bottom) with different repeat number (n=10, 15, 20, 30, 50) in molecular dynamics simulation. FIG. 2B. ESI-MS crude results obtained from synthesized (GR)₁₅ (SEQ ID NO: 11), (PR)₁₅ (SEQ ID NO: 12) and (GP)₁₅ (SEQ ID NO: 10). FIG. 2C. Line graph showing the secondary structure traces of (GP)₁₅ (SEQ ID NO: 10), (GR)₁₅ (SEQ ID NO: 11) and (PR)₁₅ (SEQ ID NO: 12) analyzed by solid FTTR. FIG. 2D. Ramachandran plots mapping the secondary structures population present in (GR)ₙ(SEQ ID NO: 19) (top) and (PR)ₙ(SEQ ID NO: 20) (bottom) with different repeat number (n=10, 15, 20, 30, 50). FIG. 2D discloses SEQ ID NOS 50-52, 17, 53, 16, 9, and 18, respectively, in order of appearance. FIG. 2E. Line graphs representing the radius gyration (Rg) changes over time in the molecular dynamics simulation of (GR)₁ (SEQ ID NO: 19) and (PR)ₙ(SEQ ID NO: 20) with different repeat number (n=10, 15, 20, 30, 50). FIG. 2F. Bar plots showing the number of R-R and H-bond (within the backbone or between backbone and R residues) interactions in (GR)ₙ(SEQ ID NO: 13) and (PR)ₙ(SEQ ID NO: 14) with different number of repeats (10, 20, 30 and 50). The values are presented as the mean±SD. FIG. 2G. All-atoms molecular dynamics simulation of the secondary structure of (GA)₁₅ (SEQ ID NO: 22). FIG. 2H. Line graph showing the changes in the distance between atoms calculated by RMSD over time in (GA)₁₅ (SEQ ID NO: 22). FIG. 2I. Ramachandran plot mapping the frequency of secondary structures present in (GA)₁₅ (SEQ ID NO: 22). FIG. 2J. Bar graphs showing coarse-grained dynamic simulation-based aggregation propensity calculated for (GR)₁ (SEQ ID NO: 13), (PR)₁ (SEQ ID NO: 14) and (GA)₁ (SEQ ID NO: 23) (n=10, 20, 30, 50). Red dashed line indicates the average AP values calculated in poly-GR. Values are presented as the mean±SD.

FIGS. 3A-3F show characterization of the interaction between R-DPRs and RNA in vitro. FIG. 3A. Photograph of turbidity assays after mixing human total RNA and synthetic (GP)₁₅ (SEQ ID NO: 10), (GR)₁₅ (SEQ ID NO: 11) and (PR)₁₅ (SEQ ID NO: 12). FIG. 3B. Line graphs depicting DPR concentration-dependent precipitation of RNA calculated by optical density. Gray shaded area indicates the DPR concentration utilized in E. FIG. 3B discloses SEQ ID NOS 11, 10 and 12, respectively, in order of appearance. FIG. 3C. Schematic showing how differential secondary structures in R-DPRs (red) can influence the number and type of interactions with human ribosomal RNA (rRNA; gray/black). Bottom: Circles showing the different types of interactions that could mediate the binding of (GR)₅₀ (SEQ ID NO: 17) and (PR)₅₀ (SEQ ID NO: 18) with rRNA. FIG. 3D. Graph bars indicating the Solvent Accessible Solvent Area (SASA) calculated by molecular dynamics simulations of (GP)ₙ (SEQ ID NO: 24), (GR)ₙ, (SEQ ID NO: 25) and (PR)ₙ(SEQ ID NO: 26) with different number of repeats (n=15, 30, 50). FIG. 3E. Line graphs depicting RNA concentration-dependent precipitation of (GR)₁₅ (SEQ ID NO: 11), (GP)₁₅ (SEQ ID NO: 10) and (PR)₁₅ (SEQ ID NO: 12) calculated by optical density measurements. FIG. 3F. Bar plots showing turbidity assay measurements utilized to assess the level of interaction of 26.7 μM of (GP)₁₅ (SEQ ID NO: 10), (GR)₁₅ (SEQ ID NO: 11) and (PR)₁₅ (SEQ ID NO: 12) with 5 μg/l ribosomal (rRNA), transfer (tRNA) and messenger (mRNA) RNA. Dot lines indicate the level of interaction of the distinct DPRs with total human RNA. All values are presented as the mean±SD; ANOVA *P<0.05; **P<0.001; ns: not significant.

FIGS. 4A-4D show characterization of the interaction between R-DPRs and RNA in vitro and localization of R-DPRs in cells. FIG. 4A. Bar plots showing turbidity assay measurements utilized to assess the level of interaction of (GR)₁₅ (SEQ ID NO: 11) with total human RNA in the absence/presence of RNase. (GP)₁₅ (SEQ ID NO: 10) was used as a reference as it does not bind to RNA. FIG. 4B. Line graphs representing the SASA changes over time in molecular dynamics simulations of (GP)ₙ (SEQ ID NO: 24), (GR)ₙ, (SEQ ID NO: 25) and (PR)ₙ(SEQ ID NO: 26) with different number of repeats (n=15, 30, 50). FIG. 4C. Left: Confocal images of HEK-293 cells transfected with GFP, GFP-(GR)₅₀, or GFP-(PR)₅₀ and labeled for the nucleolar marker fibrillarin using immunocytochemistry. Scale bar=15 μm. Right: Dot plot showing the level of colocalization of fibrillarin with GFP and GFP-fused R-DPRs. FIG. 4D. Top: Confocal images of HEK-293 cells transfected with GFP-(GR)₅₀ and immunostained for Fibrillarin or NPM1, which label the fibrillar and granular components of the nucleolus respectively. DNA was visualized by staining with Hoechst 33342. Dashed lines indicate cytosolic borders, while thin and thick pointed lines depict nuclear and nucleolar borders, respectively. Bottom: Pie charts displaying the distribution of the GFP-(GR)₅₀ signal within the different subcellular compartments (cytosol, nucleoplasm and nucleolus) defined by the selected markers. ROI: region of interest. All values are presented as the mean±standard error mean (SEM); ANOVA p*<0.05; ***<0.0001.

FIGS. 5A-5J and FIGS. 6A-6J show characterization of the interactions between poly-GR and RNA in vivo. FIG. 5A. Schematic of the CLIP-Seq procedure utilized in this study. FIG. 5B. Following UV crosslinking, cell lysis, partial ribonuclease (RNase) digestion to generate cloneable fragments and immunoprecipitation with anti-GFP antibodies (as in A.). Ribonucleoproteins (RNPs) in immunoprecipitates were labeled at the 5′ end with [γ-³²P]-ATP and fractionated using SDS-PAGE. After transfer to a membrane, RNPs were detected with autoradiography. White dashed lines: RNPs excised for library preparation. Arrow: GFP-(GR)₅₀. FIG. 5C. Pie charts showing the percentage of CLIP-Seq peaks found in various RNA classes in GFP vs. GFP-(GR)₅₀-transfected cells. Peaks were defined as having 5 or more overlapping reads. FIG. 5D. Volcano plot showing CLIP-Seq peaks enriched in GFP-(GR)₅₀ compared to GFP in CLIP-Seq. Negative log 2 (fold change) indicates peaks that were more abundant in the GFP control. Some of the most significantly enriched peaks GFP-(GR)₅₀-transfected cells are labeled. FIG. 5E. Distribution of reads mapping to the RNA45SN1 locus from the GFP-(GR)₅₀ (top) and GFP (bottom) samples. FIGS. 5F and 5G. Lysates of HEK-293 cells expressing GFP-(GR)₅₀ or GFP were subjected to immunoprecipitation with anti-GFP antibodies. RNAs within immunoprecipitates were fractionated in denaturing agarose (FIG. 5F) or polyacrylamide (FIG. 5G) gels and the indicated RNAs were detected by NB. The input represents 2% of the lysate used in immunoprecipitation. FIG. 5H. After immunoprecipitation, the levels of the indicated RNAs in anti-GFP and anti-GFP-(GR)₅₀ immunoprecipitates were measured by RT-qPCR. The fold-enrichment of each RNA relative to the anti-GFP control is shown. (n=3 independent biological replicates; Values are presented as the mean±SEM; Two-Way ANOVA, p**<0.005; ***<0.0002; ****<0.0001; ns, not significant). FIG. 5I. Schematic depicting ribosomal RNA processing and the rRNA precursors that have been detected in human cells (Henras et al., 2015). The positions of probes used for NB as well as the positions of the RT-qPCR amplicons are shown. FIG. 5J. Top: Confocal images of GFP- and GFP-(GR)₅₀-transfected HEK-293 cells immunolabeled with an anti-rRNA antibody. Hoechst 33342 was used to counterstain nuclei. Scale bar=20 μm. Right: Dot plots showing the fluorescence intensity of rRNA staining in the whole cell (left) or the nuclear/cytoplasmic ratio (right) in GFP- and GFP-(GR)₅₀-transfected HEK-293 cells. Individual cells are represented as dots. (n=3 independent biological replicates; bars represent median; Mann-Whitney U test, p****<0.0001). FIG. 6A. Lysates of HEK-293 cells transfected with GFP, GFP-(GP)₁₀ or GFP-(GR)₅₀ were subjected to WB to detect GFP. GAPDH is a loading control. FIG. 6B. After crosslinking HEK-293 cells expressing GFP-(GR)₅₀ with increasing doses of UV (254 nm) and immunoprecipitation with anti-GFP antibodies, RNPs were labeled with [γ-³²P]-ATP, fractionated by SDS-PAGE, transferred to a membrane and detected by autoradiography. Untransfected cells were controls. Arrow indicates the GFP-(GR)₅₀ band. FIG. 6C. Lysates of UV-crosslinked untransfected HEK-293 cells and cells expressing GFP-(GR)₅₀ were incubated with the indicated amounts of RNase I prior to immunoprecipitation and analysis as in (B). Arrow, GFP-(GR)₅₀. FIG. 6D. After UV crosslinking, HEK-293 cells expressing GFP, GFP-(GP)₁₀ or GFP-(GR)₅₀ were lysed, treated with the indicated amounts of RNase I and immunoprecipitated with anti-GFP antibodies. RNPs in immunoprecipitates were labeled with [γ-³²P]-ATP and detected as in (B). Dashed lines indicate RNPs excised for library preparation. Arrow indicates the GFP-(GR)₅₀ band. FIG. 6E. Pie charts indicating the percentage of CLIP-Seq peaks in the indicated classes of RNAs in GFP-(GP)₁₀ and GFP-(GR)₅₀ samples. Peaks were defined as having at least 5 overlapping reads. FIG. 6F. Distribution of reads mapping to the RNA45SN1 locus from the GFP-(GR)₅₀ (top), GFP-(GP)₁₀ (middle) and untransfected (bottom) CLIP-Seq analyses. FIGS. 6G and 611. Reads from the GFP-(GP)₁₀ and GFP-(GR)₅₀ CLIP-Seq analyses were mapped to the RNA45SN1 locus. Reads that did not map to this locus were re-mapped to the hg38 human genome to determine the fraction that mapped to other genomic loci. Reads that did not map to hg38 are designated as “unmapped”. The percentage of reads in each category was quantified as a percentage of the total reads. FIG. 61. Schematic model summarizing poly-GR interactions with rRNA and the potential consequences, including defects in ribosome biogenesis and protein translation. FIG. 6J. Left: Confocal images of in GFP- and GFP-(GR)₅₀-transfected HEK-293 cells immunolabeled for puromycin. Hoechst 33342 was used to counterstain nuclei. Scale bar=20 μm. Right: Dot plot showing the fluorescence intensity of puromycin staining in GFP- and GFP-(GR)₅₀-transfected HEK-293 cells. (n=3 independent biological replicates; bars represent median; Mann-Whitney U test, p****<0.0001).

FIGS. 7A-7D show computational modeling of poly-GR binding to ribosomal RNA and protein subunits. FIG. 7A. Top: Representative image recreated by all-atoms molecular dynamics simulations of all mammalian ribosomal proteins colored based on the number of studies that identified them as poly-GR interactors. The 28S rRNA sequence identified by our CLIP-Seq analysis is displayed in blue. Bottom: higher magnification of the mammalian ribosome proteome showing a region around the only protein that was co-precipitated with poly-GR in all 7 studies, RPL7A/eL8, which is in close proximity to the 28S rRNA sequence identified by our CLIP-Seq analysis. FIG. 7B. Representative image recreated by all-atoms molecular dynamics simulations of (GR)₅₀ (SEQ ID NO: 17) interacting with the mammalian ribosome. Based on our CLIP-Seq data and previous proteomic data (IP-MS), we determined a poly-GR interaction region (“pocket”) magnified in the inset at the bottom. The targeted RPs and RNAs were color labeled as follows: RPL7A:green; RPS30/RPL37/RPL39/RPL41:orange; 28S:blue; 5.8S:yellow; 18S:purple; Other proteins and RNAs are labeled in black and gray respectively. FIG. 7C. Line bars indicating the energetic strength of the interactions of (GR)₅₀ (SEQ ID NO: 17) in the “pocket” region of the ribosome over time. E>0 indicates repulsion; E<0 indicates attraction. The energy of interaction for 28S (blue), 5.8S (yellow), 18S (purple) and RPL7A (green) is shown by each line. Dashed line indicates the highest interaction energy achieved between (GR)₅₀ (SEQ ID NO: 17) and a 28S rRNA. FIG. 7D. Bar graphs showing average energy of interaction of (GR)₅₀ (SEQ ID NO: 17) with different ribosomal components as in C. Values are presented as mean SD.

FIGS. 8A-8E show poly-GR interaction with ribosomal proteins. FIG. 8A. Comparative data analysis of the ribosomal proteins (RPs) detected in published datasets of IP-MS experiments performed in mammalian in vitro cell models. Red dots indicate a positive interaction of poly-GR with the respective RPs. FIG. 8B. Pie charts showing the percentage of small and large ribosomal subunit proteins immunoprecipitated with poly-GR in at least one previous study. FIG. 8C. Bar graphs displaying the number of small (purple) and large (blue) ribosomal subunit proteins immunoprecipitated with poly-GR in the presence or absence of RNase as described by (Lopez-Gonzalez et al., 2016). FIG. 8D. Left: All-atoms molecular dynamics simulation of the mammalian 80S ribosome showing a putative region (indicated by a white line box) of interaction with (GR)₅₀ (SEQ ID NO: 17) (pocket). The RPs and RNAs surrounding this region are labeled as follows: RPL7A: green; RPS30/RPL37/RPL39/RPL41: orange; 28S: blue; 5.8S: yellow; 18S: purple; Other proteins and RNAs are labeled in black and gray respectively. (GR)₅₀ (SEQ ID NO: 17) is colored in red. Right: A different angle of view of the mammalian ribosome reflects the close proximity between the 28S sequence identified by our CLIP-Seq analysis and RPL7A/eL8, consistently detected as a (GR)₅₀ (SEQ ID NO: 17)-interactor by 7 independent IP-MS studies. Angle of view with respect to the image on the left is represented at the top right part of the panel. FIG. 8E. Immunoprecipitation (IP)-WB analysis of GFP-(GR)₅₀ or GFP in HEK-293 transfected cells. Cell lysates were subjected to IP with anti-GFP antibodies and WB was performed to detect RPL7A/eL8 (top) and GFP (bottom).

FIGS. 9A-9L show computational modeling of poly-GR binding to ribosomal RNA and protein subunits. FIG. 9A. Left: Line graphs showing changes in the distance between atoms calculated by root-mean-square deviation (RMSD) over time in the different components of the mammalian ribosome. Right: Line graphs showing the frequency of changes in the distance between atoms calculated by root-mean-square fluctuation (RMSF) in the different components of the mammalian ribosomal pocket region. FIG. 9B. Line graphs showing changes in the distance between atoms calculated by RMSD over time in the RNA and protein components of the “pocket” region of the mammalian ribosome in the presence of (GR)₅₀ (SEQ ID NO: 17) and (PR)₅₀ (SEQ ID NO: 18). FIG. 9C. Line graphs showing the frequency of changes in the distance between atoms calculated by RMSF in the different components of the mammalian ribosomal “pocket” region in the presence of (GR)₅₀ (SEQ ID NO: 17) and (PR)₅₀ (SEQ ID NO: 18). FIG. 9D. Representative image recreated by all-atoms molecular dynamics simulations of (GR)₅₀ (SEQ ID NO: 17) and (PR)₅₀ (SEQ ID NO: 18) interacting with distinct rRNAs and RPs present in the “no GR-RPs interaction” region magnified in the inset on the right. Based on 7 different IP-MS studies, the proteins in this ribosomal region do not interact with poly-GR. FIG. 9E. Line graphs showing changes in the distance between atoms calculated by RMSD over time in the RNA and protein components of the “no GR-RPs interaction” region of the mammalian ribosome in the presence of (GR)₅₀ (SEQ ID NO: 17) and (PR)₅₀ (SEQ ID NO: 18). FIG. 9F. Line graphs showing the frequency of changes in the distance between atoms calculated by RMSF in the RNA and protein components of the “no GR-RPs interaction” region of the mammalian ribosome in the presence of (GR)₅₀ (SEQ ID NO: 17) and (PR)₅₀ (SEQ ID NO: 18). FIG. 9G. Line graphs showing changes in the distance between atoms calculated by RMSD over time of (GR)₅₀ (SEQ ID NO: 17) and (PR)₅₀ (SEQ ID NO: 18) in the “pocket” and “no GR-RPs interaction” region of the mammalian ribosome. FIG. 9H. Line graphs showing the frequency of changes in the distance between atoms calculated by RMSF of (GR)₅₀ (SEQ ID NO: 17) and (PR)₅₀ (SEQ ID NO: 18) in the “pocket” and “no GR-RPs interaction” region of the mammalian ribosome. FIG. 9I.

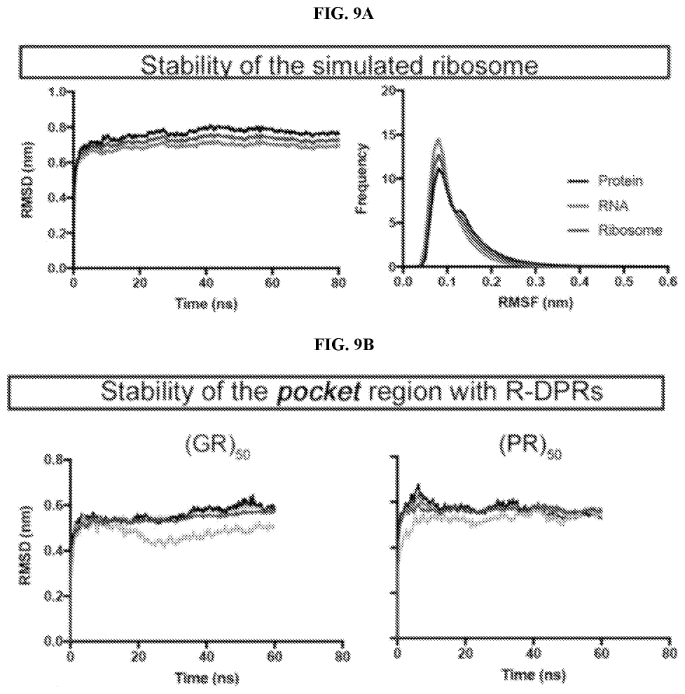

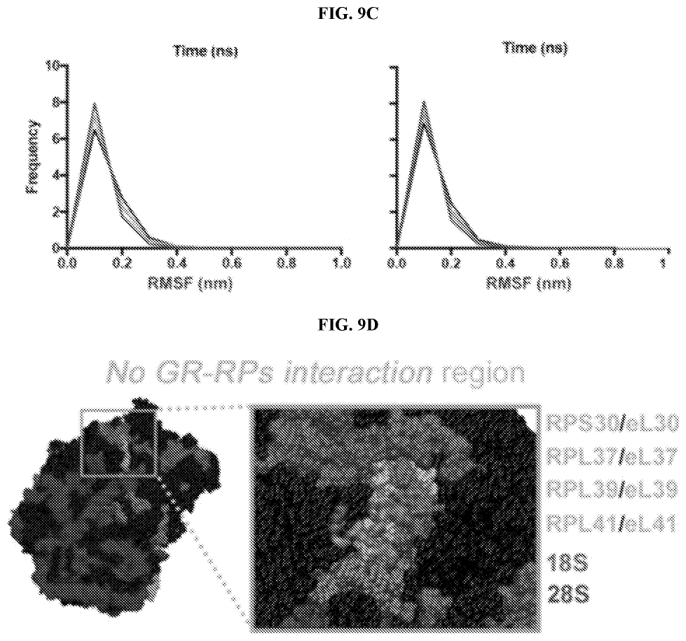

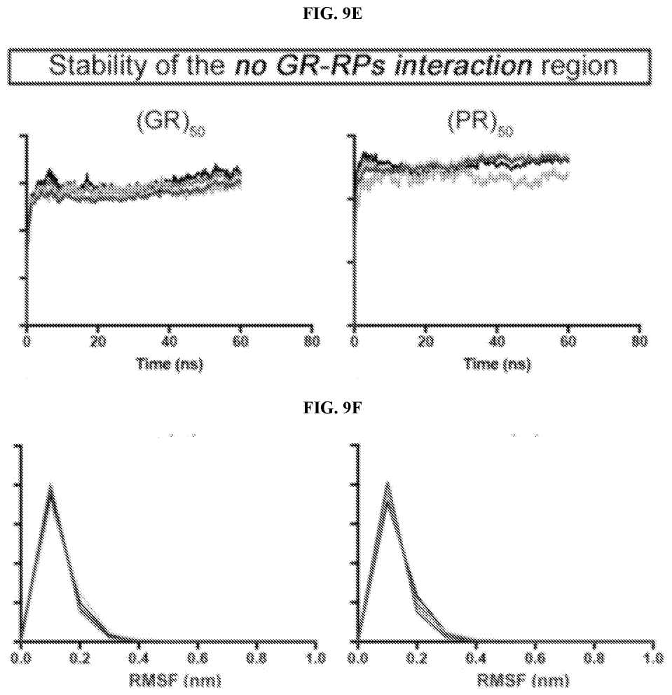

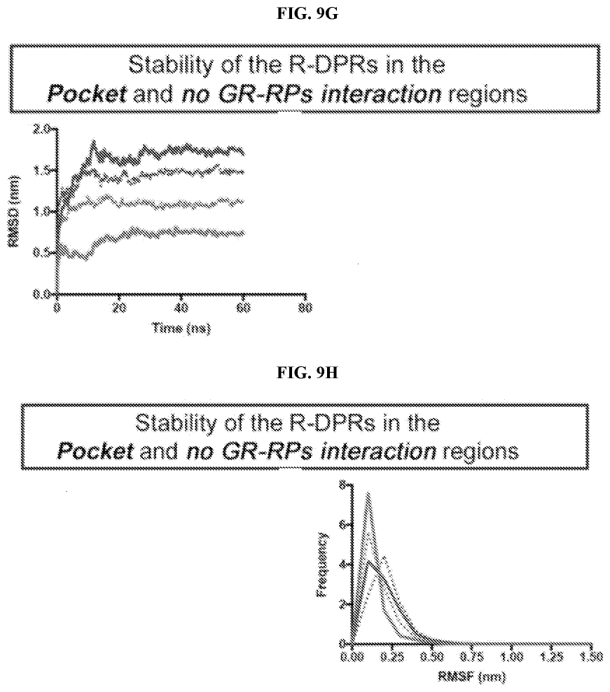

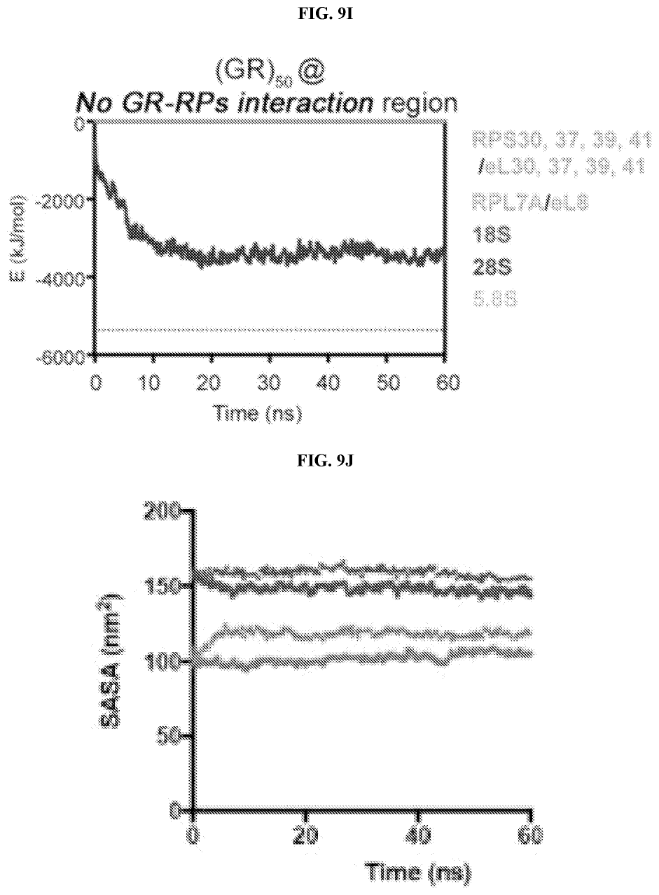

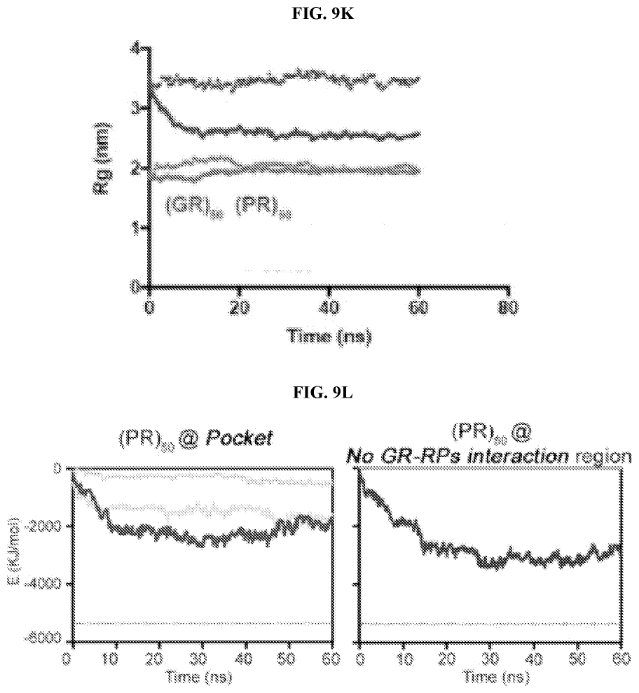

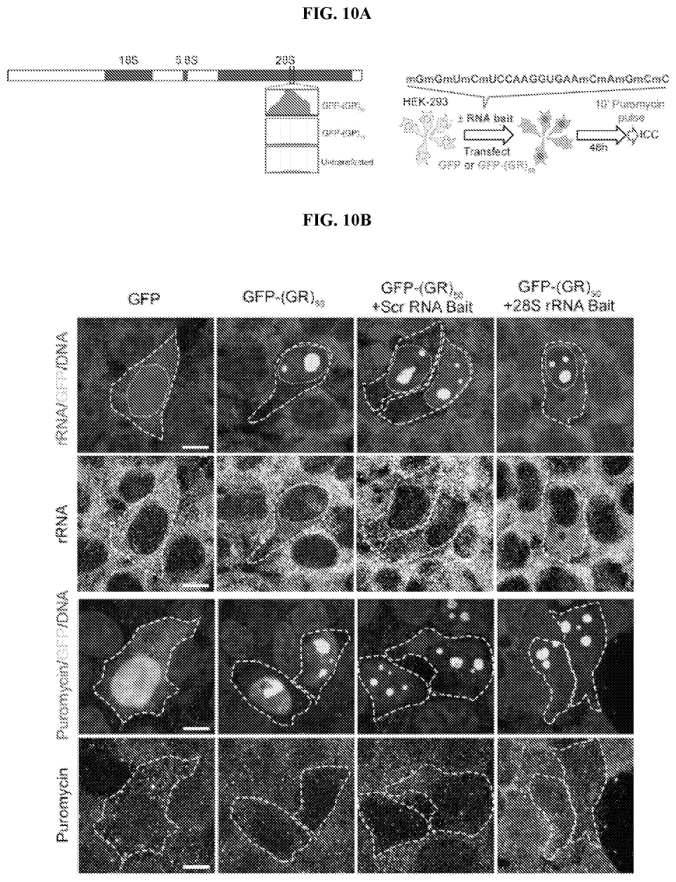

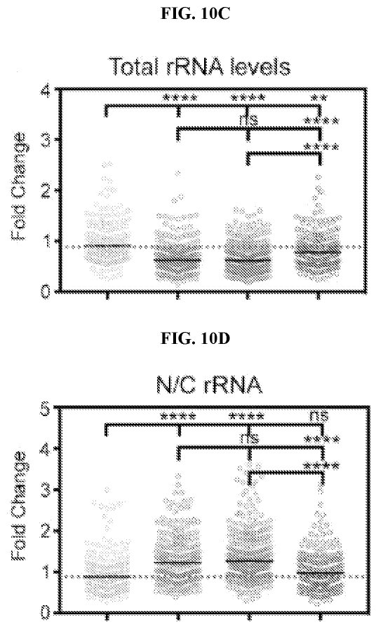

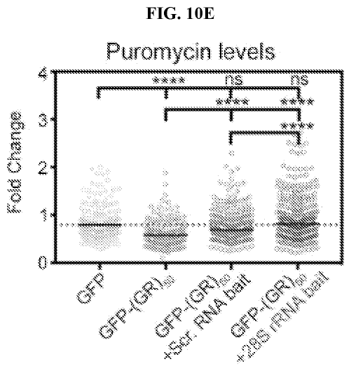

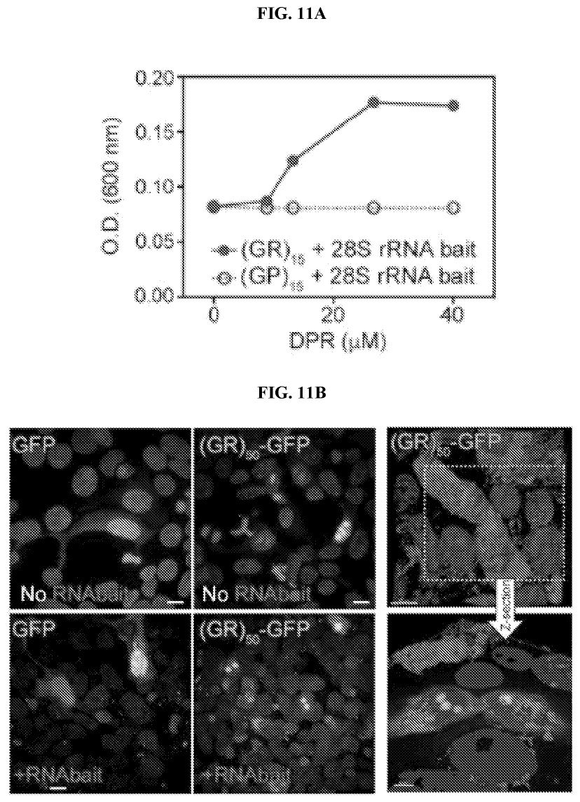

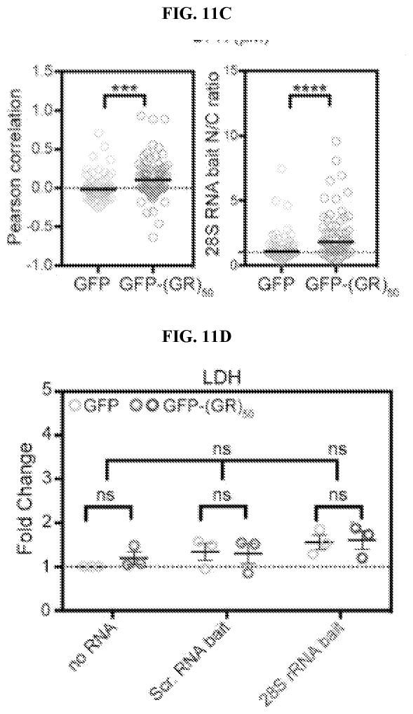

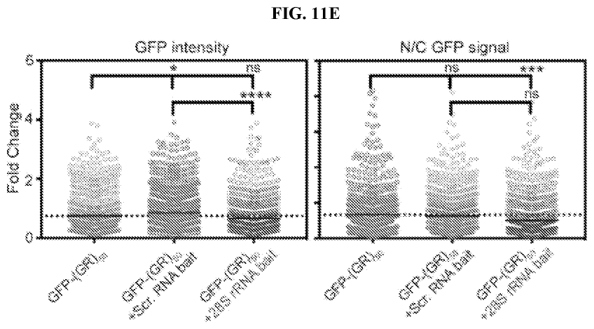

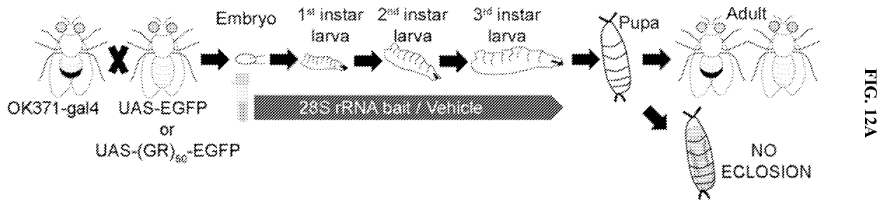

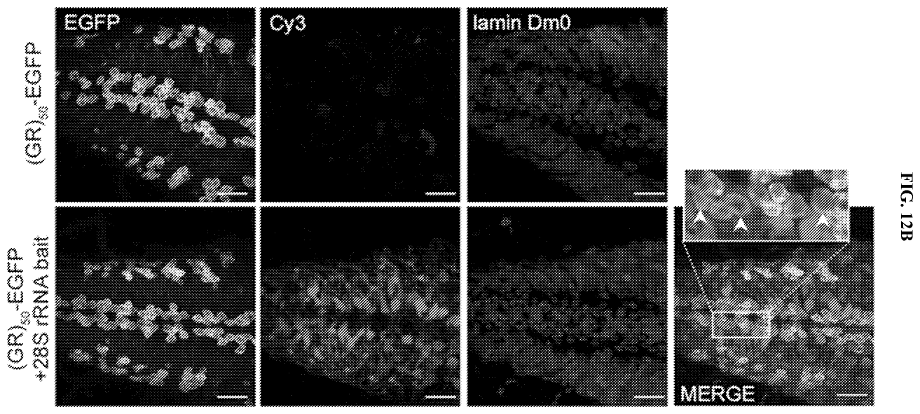

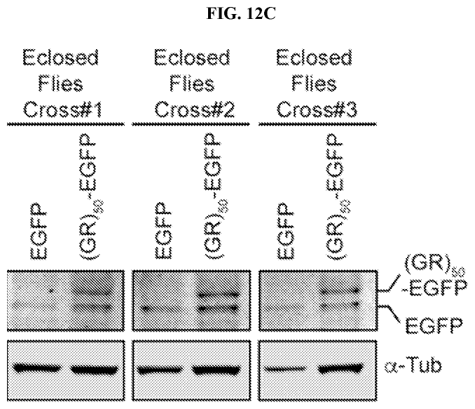

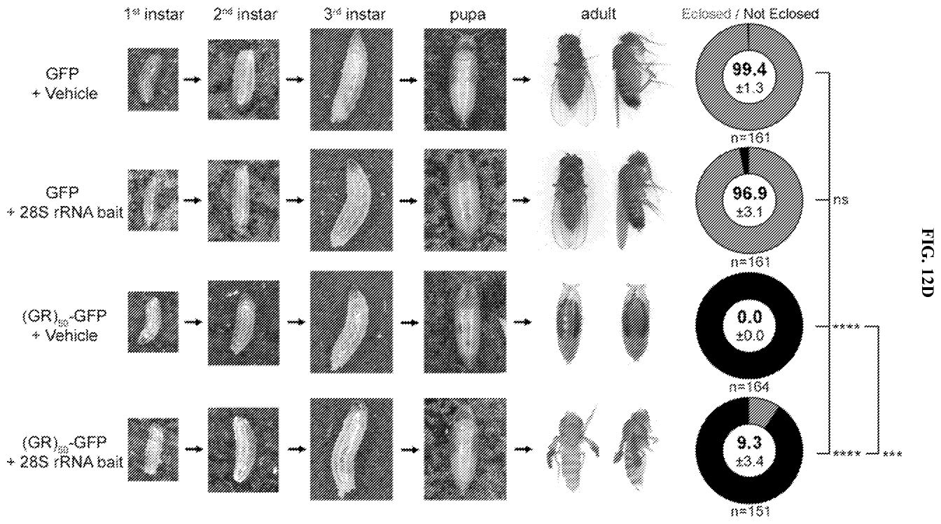

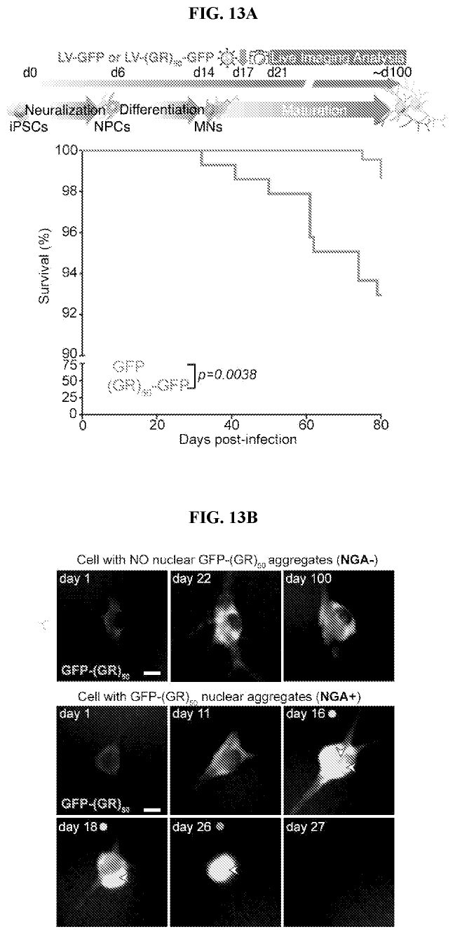

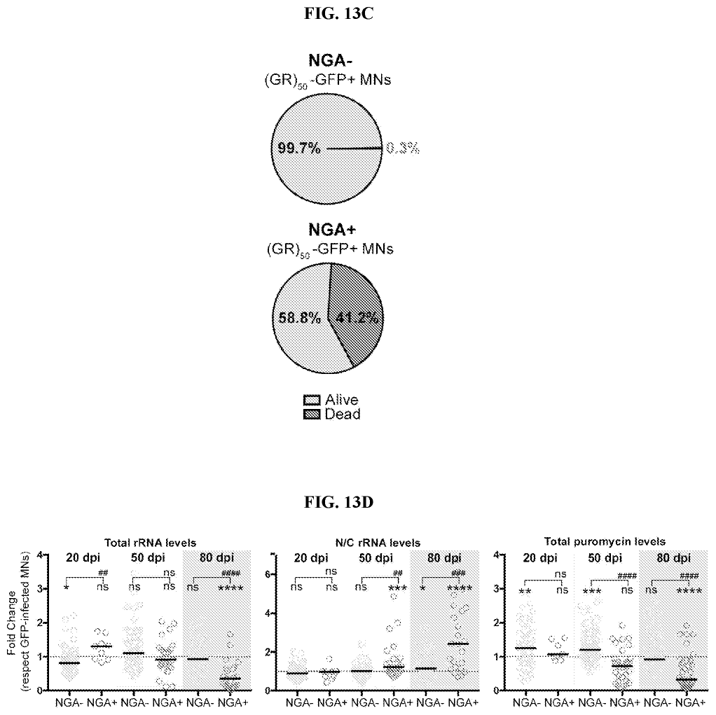

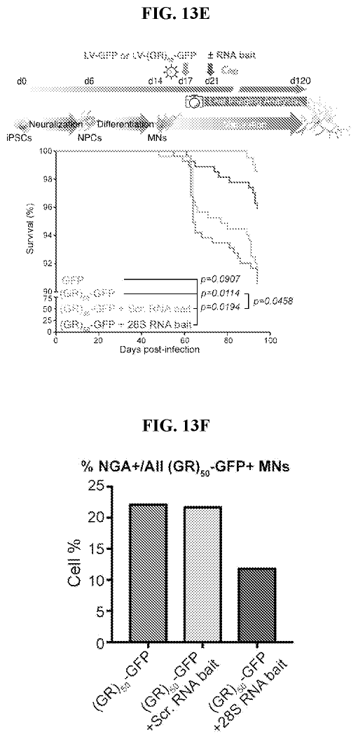

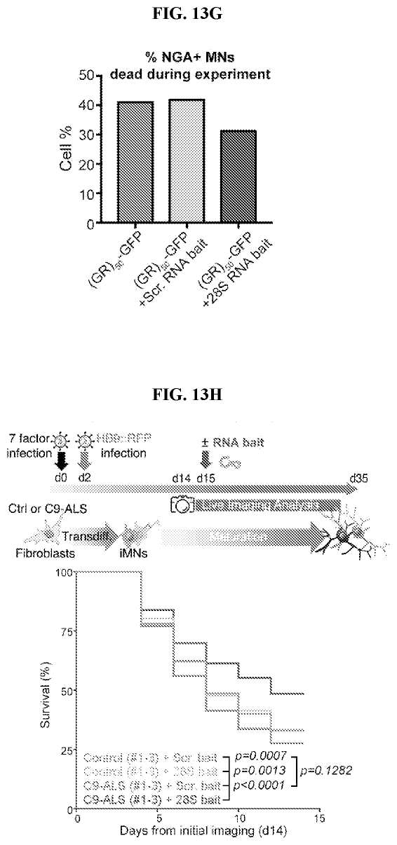

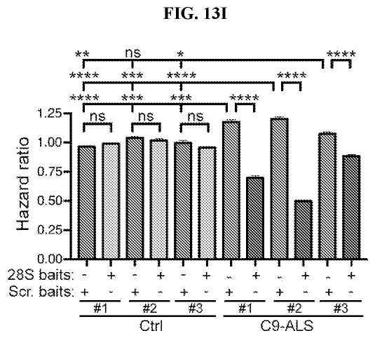

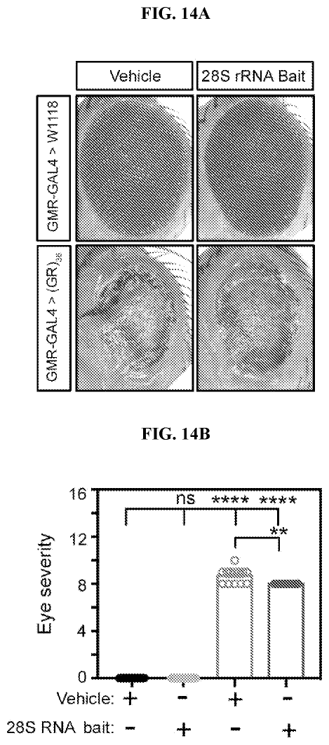

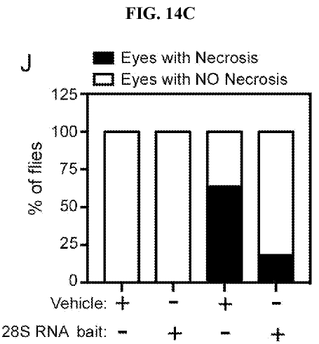

Line bars indicating the energetic strength of the interactions of (GR)₅₀ (SEQ ID NO: 17) in the “no GR-RPs interaction” region of the ribosome over time. Dashed line indicates the highest interaction energy achieved between (GR)₅₀ (SEQ ID NO: 17) and 28S rRNA. FIG. 9J. Line graphs representing the SASA value changes over time of (GR)₅₀ (SEQ ID NO: 17) and (PR)₅₀ (SEQ ID NO: 18) in the “pocket” and “no GR-RPs interaction” region of the mammalian ribosome. FIG. 9K. Line graphs representing the radius gyration changes over time of (GR)₅₀ (SEQ ID NO: 17) and (PR)₅₀ (SEQ ID NO: 18) in the “pocket” and “no GR-RPs interaction” region of the mammalian ribosome. FIG. 9L. Line bars indicating the energetic strength of the interactions of (PR)₅₀ (SEQ ID NO: 18) within the “pocket” (left) and the “no GR-RPs interaction” (right) region of the ribosome over time. Dashed line indicates the highest interaction energy achieved between (GR)₅₀ (SEQ ID NO: 17) and 28S rRNA.

FIGS. 10A-10E show modified RNA-based strategy to inhibit the toxic effects of poly-GR on ribosomal homeostasis. FIG. 10A. Left: Schematic showing the modified RNA blocking strategy based on the findings of the CLIP-Seq analysis. Right: Schematic representing the experimental workflow for rescue experiments utilizing the modified 28S rRNA baits. FIG. 10A discloses SEQ ID NO: 15. FIG. 10B. Confocal images of GFP- and GFP-(GR)₅₀-transfected HEK-293 cells immunolabeled for rRNA (top) and puromycin (bottom). Hoechst 33342 was used to counterstain nuclei. Scale bar=10 μm. FIG. 10C. Dot plots showing the fluorescence intensity of rRNA staining in GFP- and GFP-(GR)₅₀-transfected HEK-293 cells treated with scrambled or 28S rRNA-based RNA baits. FIG. 10D. Dot plots showing the N/C rRNA ratio in GFP- and GFP-(GR)₅₀-transfected HEK-293 cells treated with scrambled or 28S rRNA-based baits. FIG. 10E. Dot plots showing the fluorescence intensity of puromycin staining in GFP- and GFP-(GR)₅₀-transfected HEK-293 cells treated with scrambled or 28S rRNA-based baits. In FIGS. 10C-10E, individual cells are represented as dots and the dotted line marks the median in GFP-transfected cells. (n=3 independent biological replicates; bars represent median; Mann-Whitney U test, p**<0.01; ****<0.0001; ns, not significant).

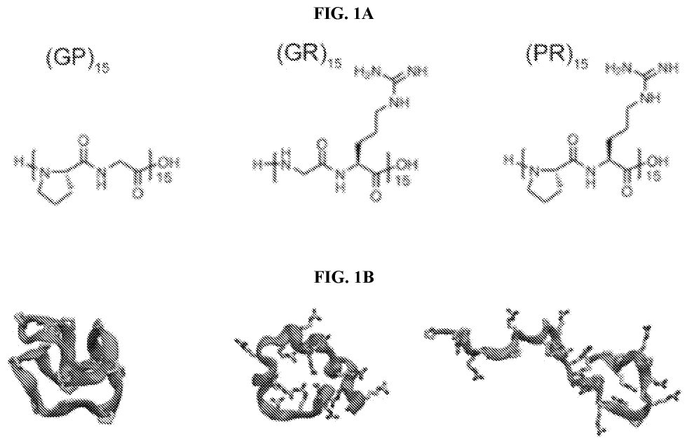

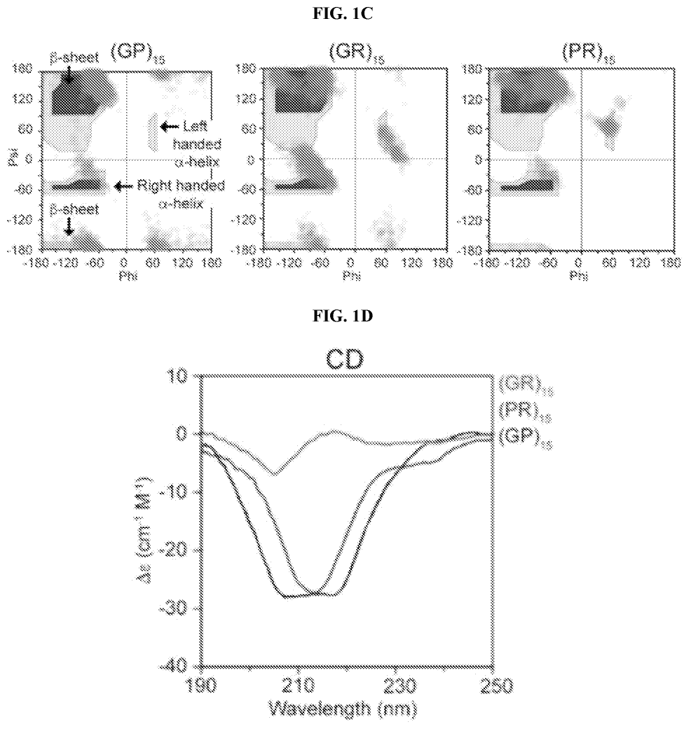

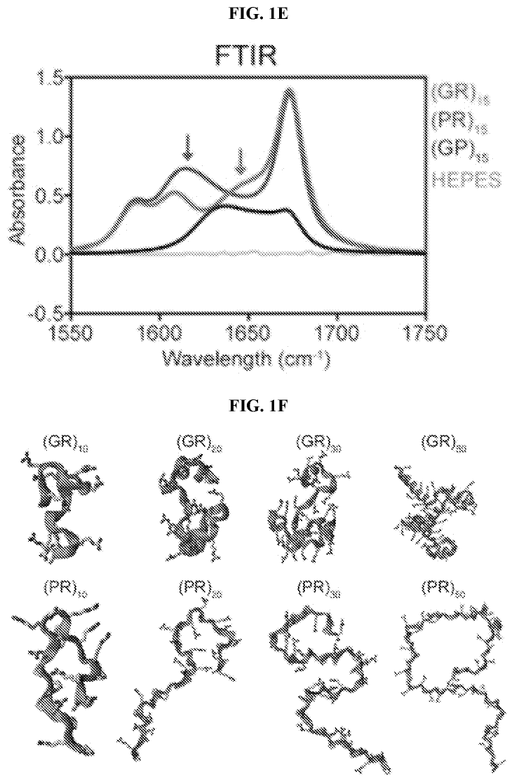

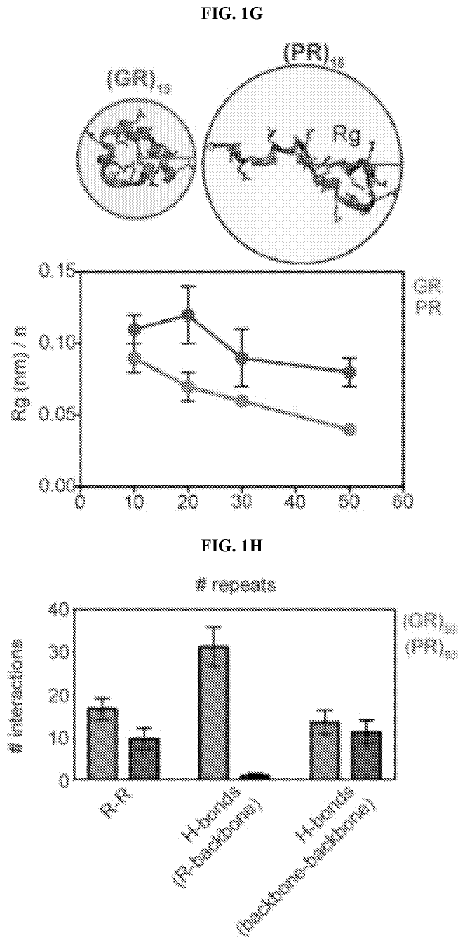

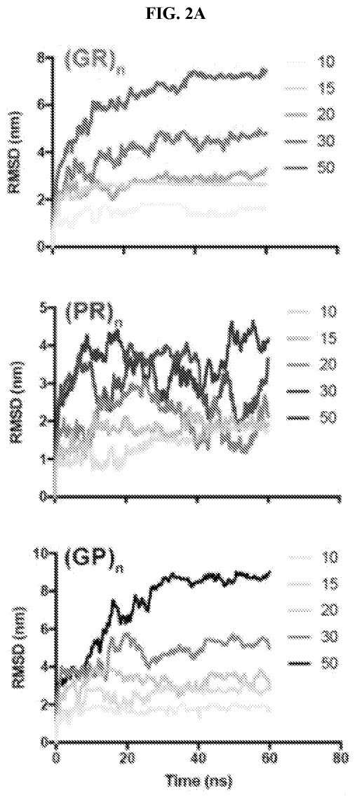

FIGS. 11A-1E show modified RNA-based strategy to inhibit the toxic effects of poly-GR on ribosomal homeostasis. FIG. 11A. Line graphs depicting DPR concentration-dependent precipitation of 28S rRNA-based baits calculated by optical density. FIG. 11A discloses SEQ ID NOS 11 and 10, respectively, in order of appearance. FIG. 11B. Left: Confocal images of HEK-293 cells sequentially co-transfected with 28S rRNA-based bait conjugated with Cy3 (red) and GFP- or GFP-(GR)₅₀ (green). Hoechst 33342 was used to counterstain nuclei. Scale bar=10 μm. Right, top: 3D-reconstruction of HEK-293 cells co-transfected with GFP-(GR)₅₀ and 28S rRNA-based bait conjugated with Cy3. Scale bar=10 μm. Inset shows a 3D shadow projection of Cy3-conjugated RNA bait in a Z-section of the cytosol and nucleus of GFP-(GR)₅₀-transfected cells (right, bottom). FIG. 11C. Dot plots showing the colocalization with GFP (left) and the nuclear/cytosolic (N/C) distribution (right) of the RNA bait conjugated with Cy3 in GFP- or GFP-(GR)₅₀-transfected HEK-293 cells. (bars represent median; Mann-Whitney U test, p***<0.001; p****<0.0001). FIG. 11D. Dot plot showing the levels of LDH levels in the media of HEK-293 cells sequentially co-transfected with scrambled or 28S-rRNA based bait and GFP- or GFP-(GR)₅₀. Average values of each experiment are represented as dots and the dotted line marks the median in no RNA bait treated GFP-transfected cells (n=3 independent biological replicates; bars represent mean±SEM; ANOVA, ns, not significant). FIG. 11E. Dot plot showing the intensity (left) and N/C distribution (right) of GFP signal in GFP-(GR)₅₀-transfected HEK-293 cells upon treatment with scrambled or 28S rRNA-based bait. Individual cells are represented as dots and the dotted line marks the median in no RNA bait-treated GFP-(GR)₅₀-transfected cells (n=3 independent biological replicates; bars represent median; Mann-Whitney U test, p*<0.05; p***<0.001; p****<0.0001; ns, not significant).

FIGS. 12A-12D show RNA-based strategy to inhibit the toxic effects of poly-GR in vivo. FIG. 12A. Schematic illustrating a Drosophila model to study poly-GR toxicity in the presence or absence of the 28S rRNA-based bait. FIG. 12B. Confocal images showing the GFP and (GR)₅₀-GFP transgene expression and the presence of 28S rRNA-based bait conjugated with Cy3 in larval brains. Lamin Dm0 immunolabeling was used as a counterstaining. Arrowheads in merged image insert display the co-localization of (GR)₅₀-GFP and 28S rRNA-based bait in larval brain cells. Scale bar=10 μm. FIG. 12C. Western blot depicting the protein expression of GFP and (GR)₅₀-GFP transgenes in brains of flies treated with 28S rRNA-based bait that eclosed. Alpha-tubulin (α-tub) was used as a loading control. FIG. 12D. Representative images of larvae, pupa and adult (OK371-gal4×EGFP) or (OK371-gal4×(GR)₅₀-EGFP) mutant flies in the presence or absence of the 28S rRNA-based bait. Pie charts on the right indicate the percentage of pupae eclosion in the different conditions. Number of animals analyzed per condition is also displayed.

FIGS. 13A-13I show RNA based-strategy to inhibit the toxic effects of poly-GR in stem cell derived MNs. FIG. 13A. Top: Top: Schematic representation of the experimental workflow for continuous live imaging analysis of iPSC-derived MNs transduced with GFP or (GR)₅₀-GFP lentiviruses. Bottom: Survival tracking by live cell imaging of iPSC-derived MNs transduced with GFP and (GR)₅₀-GFP-expressing lentivirus for >80 days after infection (dpi). Each trace includes neurons from three independent differentiations. n(GFP)=221 cells, n(GFP-(GR)₅₀)=223 cells. FIG. 13B. Fluorescence micrographs of GFP- and (GR)₅₀-GFP-transduced iPSC-derived MNs image for >80 dpi. Arrowheads indicate the presence of nuclear (GR)₅₀-GFP aggregates (NGA). Up-left green and red dot indicate the time were NGA appear and cell die, respectively. FIG. 13C. Pie charts showing the percentage of tracked (GR)₅₀-GFP-transduced iPSC-derived MNs with or without NGA that died over ˜80 dpi. FIG. 13D. Dot plot showing the fold change of total rRNA, N/C rRNA and puromycin levels in GFP-(GR)₅₀-expressing iPSC-derived MNs, with or without NGA, at 20, 50 and 80 dpi. Each dot represents the value of a single MN. Dashed lines represent the average level in control GFP-expressing MNs. Comparisons between NGA⁺ and NGA-GFP-(GR)₅₀-expressing MNs were assessed by a Mann-Whitney U test, p*<0.05, p**<0.01, p***<0.001, p****<0.0001; and between GFP-expressing MNs and NGA⁺ or NGA-GFP-(GR)₅₀-expressing MNs p^{##}<0.01, p^{###}<0.001, p^{####}<0.0001; ns, not significant. FIG. 13E. Top: Schematic representation of the experimental workflow with iPSC-derived MNs transduced with GFP or (GR)₅₀-GFP lentiviruses, which were non-treated or treated with scramble- or 28S rRNA-based baits. Bottom: Survival tracking by live cell imaging of iPSC-derived MNs transduced with GFP and (GR)₅₀-GFP-expressing lentivirus, non-treated or treated with scramble- or 28S rRNA-based baits. Each trace includes neurons from three independent differentiations. n(GFP)=206 cells, n(GFP-(GR)₅₀)=276 cells, n(GFP-(GR)₅₀+Scramble bait)=277 cells, n(GFP-(GR)₅₀+28S rRNA-based bait)=269 cells. FIG. 13F. Bar plot depicting the percentage of tracked (GR)₅₀-GFP-transduced iPSC-derived MNs, non-treated or treated with scramble- or 28S rRNA-based baits, that over ˜90 dpi displayed NGA+. FIG. 13G. Bar plot showing the percentage of tracked (GR)₅₀-GFP-transduced iPSC-derived MNs with or without NGA that died over ˜80 dpi. FIG. 13H. Top: Schematic representation of the experimental workflow with direct converted MNs (iMNs) from three control and three C9orf72-ALS (C9-ALS)-patient derived iPSC lines, which were non-treated or treated with scramble- or 28S rRNA-based baits. Bottom: Survival tracking by live cell imaging of control and C9-ALS iMNs non-treated or treated with scramble- or 28S rRNA-based baits. Each trace includes neurons from three independent iPSC lines. n=100 cells/cell line and condition. FIG. 13I. Bar plot showing the hazard ratio of control and C9-ALS iMNs non-treated or treated with scramble- or 28S rRNA-based baits. Each bar includes neurons from three independent iPSC lines. Two sample comparisons between treated vs. non-treated conditions were performed by T-test, and multiple comparisons by ANOVA; p*<0.05; p**<0.01; ***<0.001; ****<0.0001; ns, not significant.

FIGS. 14A-14C show modified RNA-based strategy to inhibit the toxic effects of poly-GR on ribosomal homeostasis. FIG. 14A. Representative eye images from (GMR-GAL4×W1118) and (GMR-GAL4×(GR)₃₆) mutant flies treated or non-treated with 28S rRNA-based bait. FIG. 14A discloses SEQ ID NO: 49. FIG. 14B. Bar graph displaying the level of eye degeneration in the different conditions as referred to H. Each dot represents values of a single fly. ANOVA test, p**<0.01; p****<0.0001; ns, not significant. FIG. 14C. Graph bar showing the percentage of (GMR-GAL4×W1118) and (GMR-GAL4×(GR)₃₆) flies with or without eye necrosis.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### Definitions

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 4, 5, 6, 7, 8, 9, 10, 11, 12, 13, 14, 15, or 16 contiguous amino acid residues of a reference peptide, respectively. Fragments may be preferentially selected from certain regions of a molecule. The term encompasses the full length polynucleotide or full length polypeptide.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the term “gene” means a segment of DNA that contains all the information for the regulated biosynthesis of an RNA product, including promoters, exons, introns, and other untranslated regions that control expression.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “peptide” refers to a polymer of amino acid residues joined by amide linkages, which may optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

As used herein, “subject” refers to an animal, such as a mammal (including a human), that has been or will be the object of treatment, observation or experiment. “Subject” and “patient” may be used interchangeably, unless otherwise indicated. Mammals include, but are not limited to, mice, rodents, rats, simians, humans, farm animals, dogs, cats, sport animals, and pets. The methods described herein may be useful in human therapy and/or veterinary applications. In some embodiments, the subject is a mammal. In some embodiments, the subject is a human.

The terms “therapeutically effective amount” and “effective amount” are used interchangibly and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or caregiver.

The term “treatment” or “treating” means administering a compound disclosed herein for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

### Inhibitory RNA Compositions of the Present Technology

In one aspect, the present disclosure provides inhibitory single-stranded RNA (ssRNA) construct that inhibit dipeptide repeat (DPR) protein-ribosomal RNA (rRNA) interaction. In some embodiments, the inhibitory ssRNA comprise a sequence that is identical to the sequence of a fragment of a ribosomal RNA (rRNA) subunit selected from the group consisting of the 28S, 18S, and 5.8S subunits, or a fragment of the 5′ETS or ITS1 region of the 47S rRNA precursor, wherein the ssRNA construct comprises one or more modified nucleotides. For non-limiting examples, the sequences of human rRNA 28S, 18S, and 5.8S subunits, and the 5′ETS and ITS1 regions of the 47S rRNA, are provided below.

Human rRNA 18S subunit. NCBI Reference Sequence NR_145820.1, Homo sapiens RNA, 18S ribosomal N1 (RNA18SN1; SEQ ID NO: 4).

Human rRNA 5.8S subunit. NCBI Reference Sequence NR_145821.1 Homo sapiens RNA, 5.8S ribosomal N1 (RNA5-8SN1; SEQ ID NO: 5).

5′ETS region of human 47S rRNA. Nucleotides 1-3654 of NCBI Reference Sequence NR_145819.1 (RNA45SN1) (SEQ ID NO: 6):

GCTGACACGCTGTCCTCTGGCGACCTGTCGCTGGAGAGGTTGGGCCTCCGGATGCGCGCGGGGCTCTGGCCTACCGGTGAC
CCGGCTAGCCGGCCGCGCTCCTGCTTGAGCCGCCTGCCGGGGCCCGCGGGCCTGCTGTTCTCTCGCGCGTCCGAGCGTCCC
GACTCCCGGTGCCGGCCCGGGTCCGGGTCTCTGACCCACCCGGGGGGGGGGGGGAAGGCGGCGAGGGCCACCGTGCCCC
CGTGCGCTCTCCGCTGCGGGCGCCCGGGGCGGCCGCGACAACCCCACCCCGCTGGCTCCGTGCCGTGCGTGTCAGGCGTTC
TCGTCTCCGCGGGGTTGTCCGCCGCCCCTTCCCCGGAGTGGGGGGTTGGCCGGAGCCGATCGGCTCGCTGGCCGGCCGGC
CGGCCTCCGCTCCCGGGGGGCTCTTCGTGATCGATGTGGTGACGTCGTGCTCTCCCGGGCCGGGTCCGAGCCGCGACGGGC
GAGGGGCGGACGTTCGTGGCGAACGGGACCGTCCTTCTCGCTCCGCCCCGCGGGGGTCCCCTCGTCTCTCCTCTCCCCGCC
CGCCGGCGGTGCGTGTGGGAAGGCGTGGGGTGCGGACCCCGGCCCGACCTCGCCGTCCCGCCCGCCGCCTTCTGCGTCGC
GGGGGGGGCCGGCGGGGTCCTCTGACGCGGCAGACAGCCCTCGCTGTCGCCTCCAGTGGTTGTCGACTTGCGGGCGGCCC
CCCTCCGCGGCGGTGGGGGTGCCGTCCCGCCGGCCCGTCGTGCTGCCCTCTCGGGGGGTTTGCGCGAGCGTCGGCTCCGC
CTGGGCCCTTGCGGTGCTCCTGGAGCGCTCCGGGTTGTCCCTCAGGTGCCCGAGGCCGAACGGTGGTGTGTCGTTCCCGCC
CCCGGCGCCCCCTCCTCCGGTCGCCGCCGCGGTGTCCGCGCGTGGGTCCTGAGGGAGCTCGTCGGTGTGGGGTTCGAGGC
GGTTTGAGTGAGACGAGACGAGACGCGCCCCTCCCACGCGGGGAAGGGCGCCCGCCTGCTCTCGGTGAGCGCACGTCCCGT
GCTCCCCTCTGGCGGGTGCGCGCGGGCCGTGTGAGCGATCGCGGTGGGTTCGGGCCGGTGTGACGCGTGCGCCGGCCGGC
CGCCGAGGGGCTGCCGTTCTGCCTCCGACCGGTCGTGTGTGGGTTGACTTCGGAGGCGCTCTGCCTCGGAAGGAAGGAGGT
GGGTGGACGGGGGGGCCTGGTGGGGTTGCGCGCACGCGCGCACCGGCCGGGCCCCCGCCCTGAACGCGAACGCTCGAGG
TGGCCGCGCGCAGGTGTTTCCTCGTACCGCAGGGCCCCCTCCCTTCCCCAGGCGTCCCTCGGCGCCTCTGCGGGCCCGAGG
AGGAGCGGCTGGCGGGTGGGGGGAGTGTGACCCACCCTCGGTGAGAAAAGCCTTCTCTAGCGATCTGAGAGGCGTGCCTTG
GGGGTACCGGATCCCCCGGGCCGCCGCCTCTGTCTCTGCCTCCGTTATGGTAGCGCTGCCGTAGCGACCCGCTCGCAGAGG
ACCCTCCTCCGCTTCCCCCTCGACGGGGTTGGGGGGGAGAAGCGAGGGTTCCGCCGGCCACCGCGGTGGTGGCCGAGTGC
GGCTCGTCGCCTACTGTGGCCCGCGCCTCCCCCTTCCGAGTCGGGGGAGGATCCCGCCGGGCCGGGCCCGGCGTTCCCAGC
GGGTTGGGACGCGGCGGCCGGCGGGCGGTGGGTGTGCGCGCCCGGCGCTCTGTCCGGCGCGTGACCCCCTCCGCCGCGA
GTCGGCTCTCCGCCCGCTCCCGTGCCGAGTCGTGACCGGTGCCGACGACCGCGTTTGCGTGGCACGGGGTCGGGCCCGCCT
GGCCCTGGGAAAGCGTCCCACGGTGGGGGCGCGCCGGTCTCCCGGAGCGGGACCGGGTCGGAGGATGGACGAGAATCACG
AGCGACGGTGGTGCGGGCGTGTCGGGTTCGTGGCTGCGGTCGCTCCGGGGCCCCCGGTGGCGGGGCCCCGGGGCTCGCGA
GGCGGTTCTCGGTGGGGGCCGAGGGCCGTCCGGCGTCCCAGGCGGGGCGCCGCGGGACCGCCCTCGTGTCTGTGGCGGTG
GGATCCCGCGGCCGTGTTTTCCTGGTGGCCCGGCCGTGCCTGAGGTTTCTCCCCGAGCCGCCGCCTCTGCGGGCTCCCGGG
TGCCCTTGCCCTCGCGGTCCCCGGCCCTCGCCCGTCTGTGCCCTCTTCCCCGCCCGCCGCCCGCCGATCCTCTTCTTCCCCC
CGAGCGGCTCACCGGCTTCACGTCCGTTGGTGGCCCCGCCTGGGACCGAACCCGGCACCGCCTCGTGGGGCGCCGCCGCC
GGCCACTGATCGGCCCGGCGTCCGCGTCCCCCGGCGCGCGCCTTGGGGACCGGGTCGGTGGCGCCCCGCGTGGGGCCCG
GTGGGCTTCCCGGAGGGTTCCGGGGGTCGGCCTGCGGCGCGTGCGGGGGAGGAGACGGTTCCGGGGGACCGGCCGCGACT
GCGGCGGCGGTGGTGGGGGCAGCCGCGGGGATCGCCGAGGGCCGGTCGGCCGCCCCGGGTGCCGCGCGGTGCCGCCGG
CGGCGGTGAGGCCCCGCGCGTGTGTCCCGGCCGCGGTCGGCCGCGCTCGAGGGGTCCCCGTGGCGTCCCCTTCCCCGCCG
GCCGCCTTTCTCGCGCCTTCCCCGTCGCCCCGGCCTCGCCCGTGGTCTCTCGTCTTCTCCCGGCCCGCTCTTCCGAACCGGG
TCGGCGCGTCCCCCGGGTGCGCCTCGCTTCCCGGGCCTGCCGCGGCCCTTCCCCGAGGCGTCCGTCCCGGGCGTCGGCGT
CGGGGAGAGCCCGTCCTCCCCGCGTGGCGTCGCCCCGTTCGGCGCGCGCGTGCGCCCGAGCGCGGCCCGGTGGTCCCTGC
CGGACAGGCGTTCGTGCGACGTGTGGCGTGGGTCGACCTCCGCCTTGCCGGTCGCTCGCCCTTTCCCCGGGTCGGGGGGTG
GGGCCCGGGCCGGGGCCTCGGCCCCGGTCGCGGTCCCCCGTCCCGGGGGGGGGGGGCGCGCCGGCCGGCCTCGGTCG
GCCCTCCCTTGGCCGTCGTGTGGCGTGTGCCACCCCTGCGCCCGCGCCCGCCGGCGGGGCTCGGAGCCGGGCTTCGGCCG
GGCCCCGGGCCCTCGACCGGACCGGTGCGCGGGCGCTGCGGCCGCACGGCGCGACTGTCCCCGGGCCGGGCACCGCGGT
CCGCCTCTCGCTCGCCGCCCGGACGTCGGGGCCGCCCCGCGGGGGGGGCGGAGCGCCGTCCCCGCCTCGCCGCCGCCCG
CGGGCGCCGGCCGCGCGCGCGCGCGCGTGGCCGCCGGTCCCTCCCGGCCGCCGGGCGCGGGTCGGGCCGTCCGCCTCCT
CGCGGGGGGGGCGACGAAGAAGCGTCGCGGGTCTGTGGCGCGGGGCCCCGGTGGTCGTGTCGCGTGGGGGGCGGGTGG
TTGGGGCGTCCGGTTCGCCGCGCCCCGCCCCGGCCCCACCGGTCCCGGCCGCCGCCCCCGCGCCCGCTCGCTCCCTCCCG
TCCGCCCGTCCGCGGCCCGTCCGTCCGTCCGTCGTCCTCCTCGCTTGCGGGGCGCCGGGCCCGTCCTCGCGAGGCCCCCCG
GCCGGCCGTCCGGCCGCGTCGGGGCCTCGCCGCGCTCTACCTTACC

ITS1 region of human 47S rRNA. Nucleotides 5524-6756 of NCBI Reference Sequence NR_145819.1 (RNA45SN1) (SEQ ID NO: 7):

ACGGAGCCCGGAGGGCGAGGCCCGCGGCGGCGCCGCCGCCGCCGCGCGCTTCCCTCCGCACACCCACCCCCCCACCGCGA
CGCGGCGCGTGCGCGGGGGGGGCCCGCGTGCCCGTTCGTTCGCTCGCTCGTTCGTTCGCCGCCCGGCCCCGCCGGCCGCG
AGAGCCGGAGAACTCGGGAGGGAGACGGGGGAGAGAGAGAGAGAGAGAGAAAGAGAAAGAAGGGCGTGTCGTTGGTGTGC
GCGTGTCGTGGGGCCGGCGGGCGGCGGGGAGCGGTCCCCGGCCGCGGCCCCGACGACGTGGGTGTCGGCGGGCGCGGG
GGCGGTTCTCGGCGGCGTCGCGGCGGGTCTGGGGGGGTCTCGGTGCCCTCCTCCCCGCCGGGGCCCGTCGTCCGGCCCCG
CCGCGCCGGCTCCCCGTCTTCGGGGCCGGCCGGATTCCCGTCGCCTCCGCCGCGCCGCTCCGCGCCGCCGGGCACGGCCC
CGCTCGCTCTCCCCGGCCTTCCCGCTAGGGCGTCTCGAGGGTCGGGGGCCGGACGCCGGTCCCCTCCCCCGCCTCCTCGTC
CGCCCCCCCGCCGTCCAGGTACCTAGCGCGTTCCGGCGCGGAGGTTTAAAGACCCCTTGGGGGGATCGCCCGTCCGCCCGT
GGGTCGGGGGCGGTGGTGGGCCCGCGGGGGAGTCCCGTCGGGAGGGGCCCGGCCCCTCCCGCGCCTCCACCGCGGACTC
CGCTCCCCGGCCGGGGCCGCGCCGCCGCCGCCGCCGCGGCGGCCGTCGGGTGGGGGCTTTACCCGGCGGCCGTCGCGCG
CCTGCCGCGCGTGTGGCGTGCGCCCCGCGCCGTGGGGGGGGAACCCCCGGGCGCCTGTGGGGTGGTGTCCGCGCTCGCC
CCCGCGTGGGGGGCGCGCGCCTCCCCGTGGTGTGAAACCTTCCGACCCCTCTCCGGAGTCCGGTCCCGTTTGCTGTCTCGTC
TGGCCGGCCTGAGGCAACCCCCTCTCCTCTTGGGCGGGGGGGGGGGGGACGTGCCGCGCCAGGAAGGGCCTCCTCCCGGT
GCGTCGTCGGGAGCGCCCTCGCCAAATCGACCTCGTA

In some embodiments, the ssRNA construct comprises a sequence that is identical to the sequence of a fragment of the rRNA 28S subunit. In some embodiments, the fragment of the rRNA 28S subunit comprises all or part of the sequence of TTGAAAATCCGGGGGAGAGGGTGTAAATCTCGCGCCGGGCCGTACCCATATC CGCAGCAGGTCTCCAAGGTGAACAGCCTC (SEQ ID NO: 1). In some embodiments, the fragment of the rRNA 28S subunit includes nucleotides 2740 and 2820 of NCBI Reference Sequence: NR_145822.1, Homo sapiens RNA, 28S ribosomal N1 (RNA28SN1; SEQ ID NO: 3). SEQ ID NO: 3 is listed below:

| | |
|---|---|
| 1 | cgcgacctca gatcagacgt ggcgacccgc tgaatttaag catattagtc agcggaggag |
| 61 | aagaaactaa ccaggattcc ctcagtaacg gcgagtgaac agggaagagc ccagcgccga |
| 121 | atccccgccc cgcggcgggg cgcgggacat gtggcgtacg gaagacccgc tccccggcgc |
| 181 | cgctcgtggg gggcccaagt ccttctgatc gaggcccagc ccgtggacgg tgtgaggccg |
| 241 | gtagcggccc ccggcgcgcc gggcccgggt cttcccggag tcgggttgct tgggaatgca |
| 301 | gcccaaagcg ggtggtaaac tccatctaag gctaaatacc ggcacgagac cgatagtcaa |
| 361 | caagtaccgt aagggaaagt tgaaaagaac tttgaagaga gagttcaaga gggcgtgaaa |
| 421 | ccgttaagag gtaaacgggt ggggtccgcg cagtccgccc ggaggattca acccggcggc |
| 481 | gggtccggcc gtgtcggcgg cccggcggat ctttcccgcc ccccgttcct cccgacccct |
| 541 | ccacccgccc tcccttcccc cgccgcccct cctcctcctc cccggagggg gcgggctccg |
| 601 | gcgggtgcgg gggtgggcgg gcggggccgg gggtggggtc ggcgggggac cgtcccccga |
| 661 | ccggcgaccg gccgccgccg ggcgcatttc caccgcggcg gtgcgccgcg accggctccg |
| 721 | ggacggctgg gaaggcccgg cggggaaggt ggctcggggg gccccgtccg tccgtccgtc |
| 781 | cgtcctcctc ctcccccgtc tccgcccccc ggccccgcgt cctccctcgg gagggcgcgc |
| 841 | gggtcggggc ggcggcggcg gcggcggtgg cggcggcggc ggcggcggcg ggaccgaaac |
| 901 | cccccccgag tgttacagcc cccccggcag cagcactcgc cgaatcccgg ggccgaggga |
| 961 | gcgagacccg tcgccgcgct ctcccccctc ccggcgccca cccccgcggg gaatcccccg |
| 1021 | cgaggggggt ctcccccgcg ggggcgcgcc ggcgtctcct cgtggggggg ccgggccacc |
| 1081 | cctcccacgg cgcgaccgct ctcccacccc tcctccccgc gcccccgccc cggcgacggg |
| 1141 | gggggtgccg cgcgcgggtc ggggggcggg gcggactgtc cccagtgcgc cccgggcggg |
| 1201 | tcgcgccgtc gggcccgggg gaggttctct cggggccacg cgcgcgtccc ccgaagaggg |
| 1261 | ggacggcgga gcgagcgcac ggggtcggcg gcgacgtcgg ctacccaccc gacccgtctt |
| 1321 | gaaacacgga ccaaggagtc taacacgtgc gcgagtcggg ggctcgcacg aaagccgccg |
| 1381 | tggcgcaatg aaggtgaagg ccggcgcgct cgccggccga ggtgggatcc cgaggcctct |
| 1441 | ccagtccgcc gagggcgcac caccggcccg tctcgcccgc cgcgccgggg aggtggagca |
| 1501 | cgagcgcacg tgttaggacc cgaaagatgg tgaactatgc ctgggcaggg cgaagccaga |
| 1561 | ggaaactctg gtggaggtcc gtagcggtcc tgacgtgcaa atcggtcgtc cgacctgggt |
| 1621 | ataggggcga aagactaatc gaaccatcta gtagctggtt ccctccgaag tttccctcag |
| 1681 | gatagctggc gctctcgcag acccgacgca cccccgccac gcagttttat ccggtaaagc |
| 1741 | gaatgattag aggtcttggg gccgaaacga tctcaaccta ttctcaaact ttaaatgggt |
| 1801 | aagaagcccg gctcgctggc gtggagccgg gcgtggaatg cgagtgccta gtgggccact |
| 1861 | tttggtaagc agaactggcg ctgcgggatg aaccgaacgc cgggttaagg cgcccgatgc |
| 1921 | cgacgctcat cagaccccag aaaaggtgtt ggttgatata gacagcagga cggtggccat |
| 1981 | ggaagtcgga atccgctaag gagtgtgtaa caactcacct gccgaatcaa ctagccctga |
| 2041 | aaatggatgg cgctggagcg tcgggcccat acccggccgt cgccggcagt cgagagtgga |
| 2101 | cgggagcggc gggggcggcg cgcgcgcgcg cgcgtgtggt gtgcgtcgga gggcggcggc |
| 2161 | ggcggcggcg gcgggggtgt ggggtccttc ccccgccccc ccccccacgc ctcctcccct |
| 2221 | cctcccgccc acgccccgct ccccgccccc ggagccccgc ggacgctacg ccgcgacgag |
| 2281 | taggagggcc gctgcggtga gccttgaagc ctagggcgcg ggcccgggtg gagccgccgc |
| 2341 | aggtgcagat cttggtggta gtagcaaata ttcaaacgag aactttgaag gccgaagtgg |
| 2401 | agaagggttc catgtgaaca gcagttgaac atgggtcagt cggtcctgag agatgggcga |
| 2461 | gcgccgttcc gaagggacgg gcgatggcct ccgttgccct cggccgatcg aaagggagtc |
| 2521 | gggttcagat ccccgaatcc ggagtggcgg agatgggcgc cgcgaggcgt ccagtgcggt |
| 2581 | aacgcgaccg atcccggaga agccggcggg agccccgggg agagttctct tttctttgtg |
| 2641 | aagggcaggg cgccctggaa tgggttcgcc ccgagagagg ggcccgtgcc ttggaaagcg |
| 2701 | tcgcggttcc ggcggcgtcc ggtgagctct cgctggccct tgaaaatccg ggggagaggg |
| 2761 | tgtaaatctc gcgccgggcc gtacccatat ccgcagcagg tctccaaggt gaacagcctc |
| 2821 | tggcatgttg gaacaatgta ggtaagggaa gtcggcaagc cggatccgta acttcgggat |
| 2881 | aaggattggc tctaagggct gggtcggtcg ggctggggcg cgaagcgggg ctgggcgcgc |
| 2941 | gccgcggctg gacgaggcgc cgccgccccc cccacgcccg gggcaccccc ctcgcggccc |
| 3001 | tcccccgccc caccccgcgc gcgccgctcg ctccctcccc gccccgcgcc ctctctctct |
| 3061 | ctctctcccc cgctccccgt cctcccccct ccccggggga gcgccgcgtg ggggcggcgg |
| 3121 | cggggggaga agggtcgggg cggcaggggc cggcggcggc ccgccgcggg gccccggcgg |
| 3181 | cgggggcacg gtcccccgcg aggggggccc gggcacccgg ggggccggcg gcggcggcga |
| 3241 | ctctggacgc gagccgggcc cttcccgtgg atcgccccag ctgcggcggg cgtcgcggcc |
| 3301 | gcccccgggg agcccggcgg gcgccggcgc gccccccccc ccaccccacg tctcgtcgcg |
| 3361 | cgcgcgtccg ctgggggcgg ggagcggtcg ggcggcggcg gtcggcgggc ggcggggcgg |
| 3421 | ggcggttcgt ccccccgccc tacccccccg gccccgtccg ccccccgttc ccccctcctc |
| 3481 | ctcggcgcgc ggcggcggcg gcgggcggcg gaggggccgc gggccggtcc cccccgccgg |
| 3541 | gtccgccccc ggggccgcgg ttccgcgcgg cgcctcgcct cggccggcgc ctagcagccg |
| 3601 | acttagaact ggtgcggacc aggggaatcc gactgtttaa ttaaaacaaa gcatcgcgaa |
| 3661 | ggcccgcggc gggtgttgac gcgatgtgat ttctgcccag tgctctgaat gtcaaagtga |
| 3721 | agaaattcaa tgaagcgcgg gtaaacggcg ggagtaacta tgactctctt aaggtagcca |
| 3781 | aatgcctcgt catctaatta gtgacgcgca tgaatggatg aacgagattc ccactgtccc |
| 3841 | tacctactat ccagcgaaac cacagccaag ggaacgggct tggcggaatc agcggggaaa |
| 3901 | gaagaccctg ttgagcttga ctctagtctg gcacggtgaa gagacatgag aggtgtagaa |
| 3961 | taagtgggag gcccccggcg cccccccggt gtccccgcga ggggcccggg gcggggtccg |
| 4021 | ccggccctgc gggccgccgg tgaaatacca ctactctgat cgttttttca ctgacccggt |
| 4081 | gaggcggggg ggcgagcccc gaggggctct cgcttctggc gccaagcgcc cggccgcgcg |
| 4141 | ccggccgggc gcgacccgct ccggggacag tgccaggtgg ggagtttgac tggggcggta |
| 4201 | cacctgtcaa acggtaacgc aggtgtccta aggcgagctc agggaggaca gaaacctccc |
| 4261 | gtggagcaga agggcaaaag ctcgcttgat cttgattttc agtacgaata cagaccgtga |
| 4321 | aagcggggcc tcacgatcct tctgaccttt tgggttttaa gcaggaggtg tcagaaaagt |
| 4381 | taccacaggg ataactggct tgtggcggcc aagcgttcat agcgacgtcg ctttttgatc |
| 4441 | cttcgatgtc ggctcttcct atcattgtga agcagaattc accaagcgtt ggattgttca |
| 4501 | cccactaata gggaacgtga gctgggttta gaccgtcgtg agacaggtta gttttaccct |
| 4561 | actgatgatg tgttgttgcc atggtaatcc tgctcagtac gagaggaacc gcaggttcag |
| 4621 | acatttggtg tatgtgcttg gctgaggagc caatggggcg aagctaccat ctgtgggatt |
| 4681 | atgactgaac gcctctaagt cagaatcccg cccaggcgga acgatacggc agcgccgcgg |
| 4741 | agcctcggtt ggcctcggat agccggtccc ccgcctgtcc ccgccggcgg gccgcccccc |
| 4801 | cctccacgcg ccccgcgcgc gcgggagggc gcgtgccccg ccgcgcgccg ggaccggggt |
| 4861 | ccggtgcgga gtgcccttcg tcctgggaaa cggggcgcgg ctggaaaggc ggccgccccc |
| 4921 | tcgcccgtca cgcaccgcac gttcgtgggg aacctggcgc taaaccattc gtagacgacc |
| 4981 | tgcttctggg tcggggtttc gtacgtagca gagcagctcc ctcgctgcga tctattgaaa |
| 5041 | gtcagccctc gacacaaggg tttgtc |

In some embodiments, the ssRNA construct comprises a sequence that is identical to the sequence of a fragment of the rRNA 5.8S subunit. In some embodiments, the fragment of the rRNA 5.8S subunit comprises all or part of the sequence ACTTCGAACGCACTTGCGGCCCCGGGTTCCTCCCGGGGCTACGCCTGTCTGAG CGTCGCTT (SEQ ID NO: 2).

In some embodiments, the ssRNA construct disclosed herein includes any form of a ssRNA having substantial homology to SEQ ID NO: 1 or SEQ ID NO: 2. In some embodiments, a ssRNA which is “substantially homologous” is about 50% homologous, more preferably about 70% homologous, even more preferably about 80% homologous, more preferably about 90% homologous, even more preferably, about 95% homologous, and even more preferably about 99% homologous to SEQ ID NO: 1 or SEQ ID NO:2.

In some embodiments, the fragment consists of up to 30 nucleotides. In some embodiments, the fragment consists of 10, 11, 12, 13, 14, 15, 16, 17, 18, 19, 20, 21, 22, 23, 24, 25, 26, 27, 28, 29, or 30 nucleotides.

In some embodiments, the ssRNA construct comprise modified nucleotides. Modification may include but not limited to 2′-modification of the ribose sugars of the modified nucleotides. In some embodiments, the modified nucleotides are 2′-O-alkyl nucleotides, 2′-deoxy-2′-fluoro nucleotides, 2′-deoxy nucleotides, 2′-H (deoxyribonucleotides), or a combination thereof. In some embodiments, the modified nucleotides are 2′-O-methyl nucleotides. In some embodiments, the modified nucleotides are at the 5′ end and/or the 3′ end of the ssRNA construct. In a specific embodiment, the ssRNA construct comprises a structure of mGmGmUmCmUrCrCrArArGrGrUrGrArAmCmAmGmCmC (SEQ ID NO: 8).

In some embodiments, the ssRNA construct may further comprise a covalently or non-covalently linked fluorophore.

In some embodiments, the ssRNA construct is formulated as a pharmaceutically acceptable composition when combined with at least one pharmaceutically acceptable carrier and/or excipient.

### Therapeutic Methods

One aspect of the present technology includes a method for treating or preventing a disease or condition characterized by elevated expression of dipeptide repeat proteins and/or the aggregates thereof as compared to that observed in a healthy subject in a subject thereof, comprising administering to the subject a therapeutically effective amount of a ssRNA construct that inhibits dipeptide repeat (DPR) protein-ribosomal RNA (rRNA) interaction, wherein the ssRNA construct comprises a sequence that is identical to the sequence of a fragment of a ribosomal RNA (rRNA) or a rRNA precursor. A disease or condition characterized by elevated expression of dipeptide repeat proteins and/or the aggregates may be but not limited to amyotrophic lateral sclerosis or frontotemporal dementia.

In some embodiments, the subject is diagnosed as having, suspected as having, or at risk of having a disease or condition characterized by elevated expression of dipeptide repeat proteins and/or the aggregates thereof as compared to that observed in a healthy subject (e.g., amyotrophic lateral sclerosis or frontotemporal dementia). The dipeptide repeat proteins may include one or more of poly-glycine-proline (polyGP), poly-glycine-alanine (polyGA), poly-glycine-arginine (polyGR), poly-proline-arginine (polyPR), and poly-proline-alanine (polyPA). In some embodiments, the subject has an increased expression of poly-GR and/or poly-PR.

In some embodiments, the subject harbors a heterozygous intronic hexanucleotide (GGGGCC)ₙ repeat expansion in C9ORF72 (C9-HRE) gene. In some embodiments, the subject exhibits one or more signs of impaired ribosomal homeostasis and function selected from the group consisting of a reduced total rRNA level, an increased nucleocytoplasmic (N/C) ratio of rRNA, or a decreased level of de novo protein translation as compared to that observed in a healthy subject. Subjects suffering from a disease or condition characterized by elevated expression of dipeptide repeat proteins and/or the aggregates thereof (e.g., amyotrophic lateral sclerosis or frontotemporal dementia) can be identified by any or a combination of diagnostic or prognostic assays known in the art.

In some embodiments of the methods disclosed herein, the ssRNA comprises one or more modified nucleotides. In some embodiments, the modified nucleotides are at the 5′ end and/or the 3′ end of the ssRNA construct.

In some embodiments, the modified nucleotides comprise 2′-modified ribose sugars. In some embodiments, the modified nucleotides are 2′-O-alkyl nucleotides, 2′-deoxy-2′-fluoro nucleotides, 2′-deoxy nucleotides, 2′-H (deoxyribonucleotides), or a combination thereof. In some embodiments, the modified nucleotides are 2′-O-methyl nucleotides.

In some embodiments, the fragment consists of up to 30 nucleotides. In some embodiments, the fragment consists of 10, 11, 12, 13, 14, 15, 16, 17, 18, 19, 20, 21, 22, 23, 24, 25, 26, 27, 28, 29, or 30 nucleotides.

In some embodiments, the ssRNA construct further comprises a covalently linked fluorophore.

In some embodiments, the ssRNA construct comprises a sequence that is identical to the sequence of a fragment of a ribosomal RNA (rRNA) subunit selected from the group consisting of the 28S, 18S, and 5.8S subunits, or a fragment of the 5′ETS or ITS1 region of the 47S rRNA precursor.

In some embodiments, the ssRNA construct comprises a sequence that is identical to the sequence of a fragment of the rRNA 28S subunit. In some embodiments, the fragment of the rRNA 28S subunit comprises all or part of the sequence of TTGAAAATCCGGGGGAGAGGGTGTAAATCTCGCGCCGGGCCGTACCCATATC CGCAGCAGGTCTCCAAGGTGAACAGCCTC (SEQ ID NO: 1). In some embodiments, the fragment of the rRNA 28S subunit includes nucleotides 2798 and 2818.

In some embodiments, the ssRNA construct comprises a sequence that is identical to the sequence of a fragment of the rRNA 5.8S subunit. In some embodiments, the fragment of the rRNA 5.8S subunit comprises all or part of the sequence ACTTCGAACGCACTTGCGGCCCCGGGTTCCTCCCGGGGCTACGCCTGTCTGAG CGTCGCTT (SEQ ID NO: 2).

In some embodiments, the ssRNA construct disclosed herein includes any form of a ssRNA having substantial homology to SEQ ID NO: 1 or SEQ ID NO: 2. In some embodiments, a ssRNA which is “substantially homologous” is about 50% homologous, more preferably about 70% homologous, even more preferably about 80% homologous, more preferably about 90% homologous, even more preferably, about 95% homologous, and even more preferably about 99% homologous to SEQ ID NO: 1 or SEQ ID NO:2.

In a specific embodiment, the ssRNA construct comprises a structure of mGmGmUmCmUrCrCrArArGrGrUrGrArAmCmAmGmCmC (SEQ ID NO: 8).

In some embodiments, the ssRNA construct inhibits polyGR-rRNA interaction.

In some embodiments, subjects with a disease or condition characterized by elevated expression of dipeptide repeat proteins and/or the aggregates thereof (e.g., amyotrophic lateral sclerosis or frontotemporal dementia) that are treated with the ssRNA construct will exhibit one or more signs of improved ribosomal homeostasis and function selected from the group consisting of a increased total rRNA level, a decreased nucleocytoplasmic (N/C) ratio of rRNA, or a increased level of de novo protein translation as compared to that observed prior to the treatment.

In some embodiments, the treatment with the ssRNA construct prevent, ameliorate, or delay the onset of one or more of the symptoms of amyotrophic lateral sclerosis selected from the group consisting of uscle twitches in the arm, leg, shoulder, or tongue; muscle cramps; tight and stiff muscles (spasticity); muscle weakness affecting an arm, a leg, the neck, or diaphragm; slurred and nasal speech; difficulty chewing or swallowing; and dicciculty moving, swallowing (dysphagia), speaking or forming words (dysarthria), or breathing (dyspnea).

In some embodiments, the treatment with the ssRNA construct prevent, ameliorate, or delay the onset of one or one more of the symptoms of frontotemporal dementia selected from the group consisting of behavior and/or dramatic personality changes, such as swearing, stealing, increased interest in sex, or a deterioration in personal hygiene habits; socially inappropriate, impulsive, or repetitive behaviors; impaired judgment; apathy; lack of empathy; decreased self awareness; loss of interest in normal daily activities; emotional withdrawal from others; loss of energy and motivation; inability to use or understand language (e.g., difficulty naming objects, expressing words, or understanding the meanings of words); hesitation when speaking; less frequent speech; distractibility; trouble planning and organizing; frequent mood changes; agitation; and increasing dependence.

In some embodiments, the ssRNA construct is administered orally, topically, intranasally, systemically, intravenously, subcutaneously, intraperitoneally, intradermally, intraocularly, iontophoretically, transmucosally, or intramuscularly.

In some embodiments, the method further comprises separately, sequentially or simultaneously administering one or more additional therapeutic agents to the subject. Nonlimiting examples of the additional therapeutic agents include C9-HRE antisense oligonucleotides (ASOs), antioxidants, or the combination thereof.

Another aspect of the present technology provides a method for inhibiting arginine-rich dipeptide repeat (R-DPR) protein-ribosomal RNA (rRNA) interaction in a cell or a subject, comprising administering to the cell or the subject a therapeutically effective amount of a ssRNA construct comprising a sequence that is identical to the sequence of a fragment of a ribosomal RNA (rRNA) or a rRNA precursor. The ssRNA construct may be any of the foregoing ssRNA constructs.

Another aspect of the present technology provides a method for restoring impaired ribosomal homeostasis and function associated with dipeptide repeat (DPR) protein expression, comprising administering to the subject a therapeutically effective amount of a ssRNA construct that inhibits arginine-rich dipeptide repeat (R-DPR) protein-ribosomal RNA (rRNA) interaction, wherein the ssRNA construct comprises a sequence that is identical to the sequence of a fragment of a ribosomal RNA (rRNA) or a rRNA precursor. The ssRNA construct may be any of the foregoing ssRNA constructs.

Another aspect of the present technology provides a method a method of determining whether a subject with amyotrophic lateral sclerosis or frontotemporal dementia is responding to treatment with a arginine-rich dipeptide repeat (R-DPR) protein-ribosomal RNA (rRNA) interaction inhibitor, comprising
detecting in a post-treatment sample obtained from a subject with amyotrophic lateral sclerosis or frontotemporal dementia that has been administered at least one dose of R-DPR protein-rRNA interaction inhibitor one or more of total rRNA level, nucleocytoplasmic (N/C) ratio of rRNA, or level of de novo protein translation, and
comparing one or more of the total rRNA level, the nucleocytoplasmic (N/C) ratio of rRNA, or the protein translation to a baseline level of total rRNA, nucleocytoplasmic (N/C) ratio of rRNA, or level of de novo protein translation from a sample obtained from the same subject before treatment was commenced,
wherein one or more of an increased total rRNA level, an decreased nucleocytoplasmic (N/C) ratio of rRNA, or an increased protein translation is indicative of the subject responding to treatment.

### Modes of Administration and Effective Dosages

Any method known to those in the art for contacting a cell, organ or tissue with a ssRNA may be employed. Suitable methods include in vitro, ex vivo, or in vivo methods. In vivo methods typically include the administration of the ssRNA construct to a mammal, suitably a human. When used in vivo for therapy, the ssRNA construct described herein are administered to the subject in effective amounts (i.e., amounts that have desired therapeutic effect). The dose and dosage regimen will depend upon the degree of the disease state of the subject, the characteristics of the particular ssRNA construct used, e.g., its therapeutic index, and the subject's history.

The effective amount may be determined during pre-clinical trials and clinical trials by methods familiar to physicians and clinicians. An effective amount of the ssRNA construct useful in the methods may be administered to a mammal in need thereof by any of a number of well-known methods for administering pharmaceutical compounds. The inhibitors may be administered systemically or locally.

The the ssRNA construct described herein can be incorporated into pharmaceutical compositions for administration, singly or in combination, to a subject for the treatment or prevention of amyotrophic lateral sclerosis or frontotemporal dementia. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injections use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having the ssRNA construct disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the ssRNA construct disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, the ssRNA construct concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of the ssRNA construct may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Kits

The present disclosure also provides kits comprising any of the foregoing ssRNA construct. Optionally, the above described components of the kits of the present technology are packed in suitable containers and labeled for the prevention and/or treatment of amyotrophic lateral sclerosis or frontotemporal dementia.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

Unless defined otherwise, all technical and scientific terms used herein have the same meaning as commonly understood by one of ordinary skill in the art to which this present invention belongs. Although any methods and materials similar or equivalent to those described herein can also be used in the practice or testing of the present invention, representative illustrative methods and materials are described herein

### EXAMPLES

### Example 1: Methods

All-Atoms Molecular Dynamics Simulations. Peptide structures were built in Avogadro (Hanwell et al., 2012) and the ribosome structure was obtained from Oryctolagus cuniculus (PDB ID: 5LZS) (Shao et al., 2016). All simulations were carried out in GROMACS 5.0.4. (Abraham et al., 2015) using Chemistry at Harvard Macromolecular Mechanics (CHARMM) force field for all-atomistic dynamic simulations that represent all the atoms of the system (Best et al., 2012; Brooks et al., 2009; Denning et al., 2011; MacKerell et al., 1998; MacKerell et al., 2000; Vanommeslaeghe et al., 2010). Force field has been widely used for simulation studies of proteins, peptides and nucleic acids (Babaian et al., 2020; Cook et al., 2020; Kognole and MacKerell, 2020; Suomivuori et al., 2020; Zhu et al., 2012). The experimental system was set up with a constant number of molecules, pressure and temperature, in a 100 mM NaCl environment with TIP3P water (Jorgensen et al., 1983). The simulation box size was set up allowing a margin of 2 nm at each side of the ribosome or of stretched DPR. DPRs simulations were done for 60 ns. Ribosome was simulated for 100 ns. Combined DPR-ribosome systems were set up placing the equilibrated DPR of 50 repeats close to the area of interest of the equilibrated ribosome and simulated for 60 ns. Molecular projections do not show the backbone nor the hydrogens which allows a better visualization of the molecular conformation and side chains. However, the multiple calculations took into account all the molecular components. All visualizations were rendered using Visual Molecular Dynamics (VMD) package (Humphrey et al., 1996).

The following analyses were also carried out using GROMCAS 5.0.4: R_{g} (radius of gyration)≈peptide extension≈1/folding.

SASA (solvent accessible surface area): Measure the exposure of the molecule to the solvent. AP=SASA(0)/SASA(i); is a normalization to show the tendency of the distinct DPRs to aggregate (AP=1. means fully soluble, AP>1 means aggregated).

RMSD (root mean square deviation) measures deviations in the structure with time respect to a reference structure (the initial conformation). Higher RMSD values involve bigger differences respect to the initial structure. Constant RMSD means stable structures and, hence, reaching a plateau is an indicative of equilibrated simulations.

RMSF (root mean square fluctuations) measures the fluctuations of atoms in nanometers respect to a reference structure in a given time range. Measured through 10 ns in the equilibrated region gives a value of the mobility of atoms. Lower RMSF involves lower mobility and, hence, higher stability of the selected atoms in the reference position.

Simulation Procedure. All the systems were minimized using steepest decent for 50000 steps or until forces on atoms converged below 1000 pN. The systems were equilibrated in NVT ensemble at 300 K for 100 ps, and then in NPT at 300 K and 1 atm for 1 ns, adding constrains in backbone atoms. Simulations were then run for the specified time (100 ns ribosomes and 60 ns DPRs and DPR-ribosome) systems in NPT ensemble. Equilibrations and simulations used 2 fs timestep and periodic boundary conditions in the three spatial coordinates. Verlet cut-off scheme was employed for non-bonded interactions with a cut-off radius of 1.2 nm (shifting van der Waals to zero from 1.0 nm) (Verlet, 1967) and Particle Mesh Ewald for long-range electrostatics (Darden et al., 1993). Temperature was controlled using velocity rescaling algorithm (T_{T}=0.1 pS) (Bussi et al., 2007). Pressure was kept constant using Berendsen algorithm for the NPT equilibration (Berendsen et al., 1984) and Parrinello-Rahman in the simulations (both Tₚ=2 ps)(Parrinello and Rahman, 1981).

Coarse-Grained Molecular Dynamics Simulations. Aggregation propensities (AP) of the different DPRs were calculated from coarse-grained molecular dynamics simulations using the MARTINI force field (version 2.2) with coil input secondary structure (de Jong et al., 2013; Marrink et al., 2007; Monticelli et al., 2008). This model maps up to 4 heavy atoms to 1 bead in order to speed up the simulations. This force field has been previously employed to measure peptide AP (Frederix et al., 2015; Frederix et al., 2011). The result for each DPR is the average of two independent simulations at same concentration but different simulations size to account for size dependence. Systems were built with constant number of amino acids 1200 or 2400 in a cubic box of 17.1 nm or 21.6 nm of side, respectively. Final concentrations are 22, 11, 7 and 4 mM for 10, 20, 30 and 50 number of repeats, respectively. Simulation procedure. All systems were minimized using steepest descent for 5000 steps or until forces converged below 200 pN. The systems were equilibrated in NPT ensemble at 303 K and 1 atm for 1000 steps using sequentially 1, 5, 10 and 20 fs timestep. Aggregation simulations were then run for 5 μs in the same ensemble with periodic boundary conditions in the three spatial coordinates. A 1.1 nm cut-off was applied for non-bonded interactions using potential-shift for Lennard-Jones and reaction field, with a dielectric constant of 15, for electrostatics (de Jong et al., 2016). V-rescale and Berendsen algorithms were used to keep temperature (T_{T}=1.0 ps) and pressure (Tₚ=6 ps), respectively, constant (Bussi et al., 2007).

Peptide synthesis. Peptides were synthesized via standard 9-fluorenyl methoxycarbonyl (Fmoc) solid-phase peptide chemistry on Wang resin using a CEM Liberty Blue automated microwave peptide synthesizer.

Automated coupling reactions were performed using 4 eq. of Fmoc-protected amino acid, 4 eq. of N,N′-diisopropylcarbodiimide (DIC), and 8 eq. of ethyl(hydroxyimino)cyanoacetate (Oxyma pure) and removal of Fmoc groups was achieved with 20% 4-methylpiperidine in DMF. Peptides were cleaved from the resin using standard solutions of 95% TFA, 2.5% water, 2.5% triisopropylsilane (TIS) and then precipitated with cold ether to yield the crude peptide product. The crude product was purified by preparative reverse-phase high-performance liquid chromatography (RP-HPLC) using a Phenomenex Kinetex column (C18 stationary phase, 5 μm, 100 Å pore size, 30×150 mm) on a Shimadzu model prominence modular HPLC system equipped with a DGU-20A5R degassing unit, two LC-20AP solvent delivery units, a SPD-M20A diode array detector and a FRC-10A fraction collector, using H₂O/CH₃CN gradient containing 0.1% CF₃COOH (v/v) as an eluent at a flow rate of 25.0 mL/min.

Liquid Chromatography-Mass Spectrometry (LC-MS). Analytical RP-HPLC was performed at 40° C. using a Phenomenex Jupiter 4 μm Proteo 90 Å column (C12 stationary phase, 4 μm, 90 Å pore size, 1×150 mm) on an Agilent model 1200 Infinity Series binary LC gradient system, using H2O/CH3CN gradient containing 0.1% CF₃COOH (v/v) as an eluent at a flow rate of 50 μL/min. Electrospray ionization mass (ESI-mass) spectrometry was performed in positive scan mode on an Agilent model 6510 Quadrupole Time-of-Flight LC/MS spectrometer using direct injection.

Optical Density measurements. Optical density (O.D.) measurements were performed on a BioTek model Cytation 3 cell imaging multi-mode reader.

Circular Dichroism (CD). CD spectra were recorded in a Jasco J-815 spectropolarimeter using quartz cells of 100 μm pathlength. Spectra were background subtracted and are the average of three scans using continuous scanning mode at a speed of 100 nm/min and standard sensitivity. Final spectra are normalized to concentration.

Fourier-transform infrared spectroscopy (FTIR). FTIR spectra were recorded on a Bruker Tensor 37 FTIR spectrometer. Spectra shown are the average of 25 scans with a resolution of 1 cm-1. Samples were prepared in deuterated water (D₂O) to displace its vibrations from the region of interest. Liquid samples were placed between two CaF₂ windows with 50 μm pathlength and background subtracted using the solvent. Solid FTIR was measured using attenuated total reflectance (ATR) module on lyophilized samples and using background subtraction to remove signals from atmospheric H₂O and CO₂.

RNA-dipeptide repeats binding assay. The RNA solutions were diluted with HEPES buffer ([phosphate]=200 μM) and incubated for 30 min at 25° C. A HEPES buffer solution of dipeptide repeats (10 mM) was then added to the RNA solutions, and the mixture was pipetted 30 times. 50 μL of these suspensions were put into triplicate wells of a 96-well plate, and their optical density at 600 nm were recorded.

Estimation of the content of phosphates in the RNA sample. The content of phosphates in the RNA sample was estimated based on assumptions that the RNA is 100% pure and its counter cation is sodium. Molecular weight of the RNA repeating unit (343.43 g/mol) was calculated by averaging the molecular weights of adenine (351.19 g/mol), guanine (367.19 g/mol), cytosine (327.17 g/mol) and uracil (328.15 g/mol). As the concentration of RNA was shown to be 5.4 μg/μL, the concentration of phosphates was therefore estimated to be 15.7 mM.

Cell cultures and DPR overexpression system. HEK-293FT cells were grown in DMEM (Corning) supplemented with Glutamax (Gibco) and 10% fetal bovine serum (FBS, Gibco). HEK-293FT cells were dissociated by incubating for 5 min with Trypsin-EDTA (Gibco) at 37° C. Cells were maintained at 37° C., 5% CO2 without antibiotics and tested on a monthly basis for mycoplasma.

For overexpression experiments, 40% confluent HEK-293 cells were transfected with HilyMax transfection reagent (Dojindo Molecular Technologies) according to manufacturer guidelines. Briefly, DNA was mixed with HilyMax (1 μg DNA:3 μL HilyMax ratio) in Opti-MEM medium (Gibco) and incubated for 15 min at room temperature (RT) before being added to cells. Cells were incubated with transfection mixture for 4 hrs at 37° C. and then media was replaced. Analyses made on transfected cells were performed 48 to 72 hrs after transfection. For overexpression experiments, 40% confluent HEK-293 cells were transfected with HilyMax transfection reagent (Dojindo Molecular Technologies) according to manufacturer guidelines. Briefly, DNA was mixed with HilyMax (1 μg DNA:3 μL HilyMax ratio) in Opti-MEM medium (Gibco) and incubated for 15 min at room temperature (RT) before being added to cells. Cells were incubated with transfection mixture for 4 hrs at 37° C. and then media was replaced. Analyses made on transfected cells were performed 48 to 72 hrs after transfection.

Plasmids. HEK-293 cells were transfected with the pcDNA3.1 plasmid containing GFP, (GP)×10-, or (GR)×50-GFP, in which alternative codons were used to generate DPRs without generating the (GGGGCC)n transcript. These constructs were made and kindly shared by Petrucelli's lab (Zhang et al., 2014).

CLIP-Seq. CLIP experiments were performed according to Huppertz et. al. (Methods. 2014 February; 65(3): 274-287. doi: 10.1016/j.ymeth.2013.10.011). Briefly, cells were crosslinked in a Spectroline UV crosslinker using 100 J/cm² UVC (254 nm). Cells were lysed in 50 mM Tris-HCl pH 7.4, 150 mM NaCl, 1% NP-40, 0.5% sodium deoxycholate, 0.1% SDS with EDTA-free complete mini protease inhibitor cocktail (Roche) and 1 mM phenylmethylsulfonyl fluoride (PMSF). After sonicating on ice using a Branson sonifier, lysates were incubated with 4 U/ml Turbo DNase (Invitrogen) and 0.2 to 0.002 units/ml of RNase I (Invitrogen). Immunoprecipitation was performed by incubating the lysates with GFP-Trap Magnetic beads (Chromotek cat: gtd-10) for 1.5 hours at 4° C. Afterwards, beads were washed twice with high salt buffer (50 mM Tris-HCl pH 7.4, 1 M NaCl, 1% NP-40, 0.5% sodium deoxycholate, 0.1% SDS) and twice with PNK buffer (20 mM Tris-HCl pH 7.4, 10 mM MgCl2, 0.2% Tween-20). RNA 3′ ends were dephosphorylated with PNK as described (Huppertz et al.), followed by two washes each with high salt and PNK buffers. 3′ linker ligation was performed on beads overnight at 16° C. followed by two additional washes each with high salt and PNK buffer. After labeling with [γ-³²P]-ATP and T4 RNA ligase, RNPs were resolved on NuPAGE 4-12% Bis-Tris gels (Invitrogen) run in NuPAGE MOPS-SDS running buffer (Invitrogen). After gel fractionation, RNPs were transferred to Amersham Protran 0.45 uM nitrocellulose membranes (Cytiva) and labeled complexes visualized using a storage phosphor screen and developed on a Typhoon FLA 9000 scanner. RNA-protein complexes of the expected size were excised from the nitrocellulose membranes, along with the same size region from GFP, GFP-(GP)₁₀, or untransfected control experiments. Associated proteins were removed by digesting with 1 mg/ml proteinase K (Invitrogen) for 20 minutes at 37° C. in PK buffer (100 mM Tris-HCl pH7.4, 50 mM NaCl, 10 mM EDTA) followed by a second digestion for 20 minutes at 37° C. in the presence of 3.5 M urea. Afterwards, RNA was extracted with phenol chloroform and reverse transcription performed using Superscript III (Invitrogen) using the following primers: library 1 GPF—Rt1clip, GFP-(GR)₅₀—Rt6clip; library 2 untransfected—Rt1clip, GFP-(GP)₁₀—Rt6clip, GFP-(GR)₅₀—Rt9clip. cDNA was size selected and circularized with Circligase II (Epicentre). Circularized cDNA was cut with BamHI and amplified using Accuprime Supermix I (Invitrogen). The PCR cycle number was optimized to prevent over amplification of the library. Amplified samples were sequenced on the Illumina MiSeq platform in single end read mode with 110 nt reads.

CLIP-Seq bioinformatics. For mapping to the whole genome, barcoded sequencing libraries were demultiplexed allowing 1 nt barcode mismatch, and adapter sequences and low-quality bases were filtered using iCount (version 2.0) (https://github.com/tomazc/iCount). Trimmed, single-end reads were aligned to the hg38 genome (Gencode V32) using Novoalign (version 4.03.03). Parameters for alignment included: score threshold (-t) set to 15.3, the minimum number of bases for alignment (-l) set to 20, the gap penalty (-x) set to 4, gap opening penalty (-g) set to 20, trimming step size (-s) set to 1, score difference (-R) set to 0 and multimapping reporting (-R) set to ALL. The quality of the sequenced libraries was assessed per sample using FastQC (version 0.11.5) https://www.bioinformatics.babraham.ac.uk/projects/fastqc/, FastQ Screen (version 2) (Wingett and Andrews, 2018) and samtools (version 1.13) (Li et al., 2009). Samples were deduplicated using UMI-tools (Smith et al., 2017), with multi-mapping detection. On average 5.33 unique UMIs were detected per position.

To determine the location of CLIP peaks, overlapping reads were collapsed to create a list of genomic regions using bedtools (version 2.30.0) (Quinlan and Hall, 2010). Peaks found within 50 nt of one another were combined to a single feature. Reads associated with CLIP peaks were counted using FeatureCounts (version 2.0.2) (Liao et al., 2014) with the following parameters: the number of reads supporting each exon-exon junction was included, the minimum number of overlapping bases in a read required for assignment as 1, and strand specific read counting was performed. Analysis was performed without multi-mapped reads (unique reads only) and with multi-mapped reads included (unique+multi-mapped reads). Multi-mapped reads were assigned a fractional count of 1/x, where x is the total number of alignments reported for the same read. CLIP peaks with >5 unique+fractional multi-mapped reads were annotated with overlapping genomic features. Protein coding and ncRNA features were identified from the Gencode hg38 V32 while ncRNA and repeat regions were identified from hg38/GRCh38 Repeatmasker annotations and rRNA annotations were identified from RefSeq GRCh38.p13 (GCF_000001405.39). Representative peaks with multiple annotations were initially examined manually to determine the correctness of the annotations. Based on this analysis, preference was given to annotations as follows: rRNA>ncRNA>protein coding: exon>repeat element>pseudogene>lncRNA: exon>antisense feature>protein coding: intronic>lncRNA: intronic>no feature.

To identify specific sites of RNA binding, MAnorm (version 1.1.4) (Shao et al., 2012) was used to identify enriched CLIP peaks in GFP-(GR)₅₀ vs GFP samples. Default parameters were used except for the following: shift size for both inputs (--s1,--s2,) was set to 0 to keep the peak binding site at the 5′ end and the summit-to-summit distance cutoff for common peaks (-d) was set to 25 to ensure only overlapping peaks between samples were compared. 10,000 simulations (-n) were performed to test enrichment. CLIP peaks with a fold-change >2 and a p-value <0.05 were identified as significant binding events.

For mapping to the RNA45SN1 locus, PCR duplicates were removed by collapsing identical sequences using the FastX_collapser. PhiX spike in control reads were removed by mapping to Coliphage phi-X174, complete genome (NCBI Reference Sequence: NC_001422.1). The remaining reads were demultiplexed based on barcodes using FastX_splitter, then adapter and UMI sequences were removed with fastX_trimmer and fastX_clipper, respectively (Fastxtoolkit version 0.0.14) (http://hannonlab.cshl.edu/fastx_toolkit/). Bowtie2-build was used to generate a genomic index from the RNA45SN1 (NC_000021.9) sequence. Reads were mapped to this index using Bowtie2 (version 2.4.4) (http://bowtie-bio.sourceforge.net/bowtie2/index.shtml) with the -- very-sensitive-local option. Unmapped reads were extracted using Samtools-view. These unmapped reads were then mapped to the hg38 human genome using STAR. Reads mapping to 45S were counted based on the regions with which they overlapped. Bedtools intersect was used to count reads in the various regions. For precursor regions (5′ETS, ITS1, ITS2 and 3′ETS) reads with at least one nucleotide in these regions were counted. For mature rRNA regions (18S, 5.8S and 28S) only reads mapping entirely within these regions were counted. The percentage of reads mapping to each region were represented as a percentage of the total collapsed read number.

RNA immunoprecipitations (RIP) followed by detection of RNAs by Northern blot (NB) and RT-qPCR. HEK-293 cells expressing GFP- or GFP-(GR)₅₀ were lysed in NET-2 (50 mM Tris pH 7.5, 150 mM NaCl, 2.5 mM MgCl2, 0.5% NP-40) and 1 mM phenylmethylsulfonyl fluoride (PMSF). After sonicating in a Bioruptor Plus on high (30 seconds on, 30 seconds off) for 1 minute at 4 C, lysates were sedimented at 16,000×g for 10 minutes. Cleared lysates were incubated with 25μ of GFP-Trap magnetic beads for 1.5 hours at 4 C with rotation. Beads were washed twice in NET-2, transferred to a fresh tube and washed two additional times. Beads were resuspended in 400 ul NET-2 and extracted with an equal volume of acid-phenol:chloroform (Invitrogen AM9722). After centrifugation at 16,000×g for 15 minutes, RNA was precipitated from the aqueous phase by adding 1/10^{th} volume sodium acetate and 2.5 volumes 100% ethanol. After precipitation, RNA was fractionated in a 5% polyacrylamide/7M urea gel (to detect RNAs of less than 500 nts) or an 0.8% agarose/formaldehyde gel using the Tricine/Triethanolamine buffer system described by (Mansour and Pestov, 2013) to detect larger RNAs. RNA was transferred from polyacrylamide gels to Hybond-N (Cytiva) in 0.5×TBE for 16 hours at 150 mA. RNA was transferred from agarose gels to Hybond-N by capillary transfer overnight using 10×SSC (1.5 M NaCl, 150 mM sodium citrate pH 7). RNA was crosslinked to membranes using a Spectroline UV crosslinker and hybridized in modified (Church and Gilbert, 1984) hybridization buffer (1% BSA, 2 mM EDTA, 200 mM NaHlPO4 pH 7.2, 15% DI formamide, 7% SDS) using 5′-³²P labeled oligonucleotides at 28 C. Northern probe sequences used in this study:

5.8S-
(SEQ ID NO: 27)
GTGTCGATGATCAATGTGTCCTGCAATTCA
18S-
(SEQ ID NO: 28)
CGCTCCACCAACTAAGAACG
28S
(SEQ ID NO: 29)
CCTGGTTAGTTTCTTCTCCTCC
7SL-
(SEQ ID NO: 30)
CCATATTGATGCCGAACTTAGTGC
RPPH1-
(SEQ ID NO: 31)
CTGTTCCAAGCTCCGGCAAA
and
(SEQ ID NO: 32)
AATGGGCGGAGGAGAGTAGT

For RT-qPCR analyses, the RNA was reverse transcribed using the iSCRIPT cDNA Synthesis Kit (Bio-Rad) and qPCR was performed using iTaq Universal SYBR Green Supermix (biorad). Samples were run on a Bio-Rad CFX96 Real Time PCR System and analyzed using Maestro software (Bio-Rad).

Primer sequences used in this study:

28S_F-
(SEQ ID NO: 33)
GGAGGAGAAGAAACTAACCAGG
28S R-
(SEQ ID NO: 34)
GTCTTCCGTACGCCACATGTC
18S F-
(SEQ ID NO: 35)
CTCAACACGGGAAACCTCAC
18S R-
(SEQ ID NO: 28)
CGCTCCACCAACTAAGAACG
5′ ETS F-
(SEQ ID NO: 36)
TCTAGCGATCTGAGAGGCGT
5′ ETS R-
(SEQ ID NO: 37)
CAGCGCTACCATAACGGAGG
ITS1_F-
(SEQ ID NO: 38)
CAACCCCCTCTCCTCTTGGG
ITS1_R-
(SEQ ID NO: 39)
GAGGTCGATTTGGCGAGGG
Y4_F-
(SEQ ID NO: 40)
GGCTGGTCCGATGGTAGTGG
Y4_R-
(SEQ ID NO: 41)
AAAGCCAGTCAAATTTAGCAGTGGG
Y5_F-
(SEQ ID NO: 42)
AGTTGGTCCGAGTGTTGTGGG
Y5_R-
(SEQ ID NO: 43)
AAAACAGCAAGCTAGTCAAGCGCG
tRNA-Glu_F-
(SEQ ID NO: 44)
TCCCACATGGTCTAGCGG
tRNA-Glu_R-
(SEQ ID NO: 45)
TTCCCACACCGGGAGT

Immunoprecipitation (IP) followed by western blot (WB) analysis. Cells were harvested in IP buffer (10 mM Hepes pH 7.6, 100 mM NaCl, 1 mM EDTA, 1 mM NaF, 2 mM Na₃VO₄, 1 mM DTT, 1 mM PMSF, 1% sodium deoxycholate, 10% glycerol, 0.1% SDS, 1% Triton X-100 and 1× protease inhibitor cocktail). Lysates were sonicated and protein concentrations determined with a BCA kit (Pierce). GFP and GFP-tagged proteins were immunoprecipitated from 1 mg of protein/sample with anti-GFP antibody (Abcam). Immunoprecipitation of the target antigen was performed using Dynabeads® Protein A (Novex, life technologies) following the manufacturer's protocol (www.thermofisher.com/document-connect/document-connect.html?url=https%3A%2F%2Fassets.thermofisher.com%2FTFS-Assets%2FLSG%2Fmanuals%2FDynabeadsProteinA_man.pdf&title=RHluYWJlYWRzI FByb3Rl aW4gQQ==). Eluted proteins were separated by SDS-PAGE followed by electrotransfer to a nitrocellulose membrane (Bio-Rad). The membranes were blocked in Tris-buffered saline (TBS, 50 mM Tris, 150 mM NaCl, HCl to pH 7.6)+0.1% Tween 20 (Bio-Rad)+5% non-fat dry milk (LabScientific) and incubated overnight at 4° C. with primary antibodies: GAPDH (rabbit, 1:1000, Cell Signaling), GFP (goat, 1:1000, Abcam), RPL7A (rabbit, 1:1000, Cell Signaling Technology). Primary antibodies were diluted in TBS+0.1% Tween+5% BSA (Calbiochem). After several washes in TBS+0.1% Tween, membranes were incubated with their corresponding secondary HRP-conjugated antibodies (1:5000, LI-COR Biotechnology). Protein signals were detected by a ChemiDoc™ XRS+ (Bio-Rad), using the SuperSignal West Pico chemiluminescent system (Thermo Scientific).

Immunocytochemistry. Cells were fixed with 4% paraformaldehyde for 20 min, washed with PBS and permeabilized/blocked for 1 h in PBS containing 10% normal donkey serum (Jackson ImmunoResearch) and 0.2% Triton. Samples were then incubated overnight at 4° C. with primary antibodies: puromycin (mouse, 1:5000, Millipore), rRNA (mouse, 1:1000, Novus Biologicals), fibrillarin (rabbit, 1:2000, Abcam), NPM1 (mouse, 1:500, Santa Cruz Biotechnology). The next day, PBS+0.1% Triton was applied for several washes. Samples were then incubated with the appropriate secondary antibodies conjugated to Alexa488, Alexa555 or Alexa647 fluorophores (1:500 to 1:1000 Molecular Probes) for 1 h at RT. Cell nuclei were labeled using Hoechst 33342 (Life Technologies) to stain DNA. Immunolabeled samples were blinded upon mounting for subsequent imaging analysis.

De novo protein translation analysis. For single-cell protein translation analysis, we utilized a puromycin-based method termed SUnSET (Schmidt et al., 2009) that labels newly synthesized proteins. In short, cell cultures were pulsed for 5-10 min with puromycin (20 μM) at 37° C. Cells were then fixed and immunocytochemistry with anti-puromycin antibody was carried out as described above.

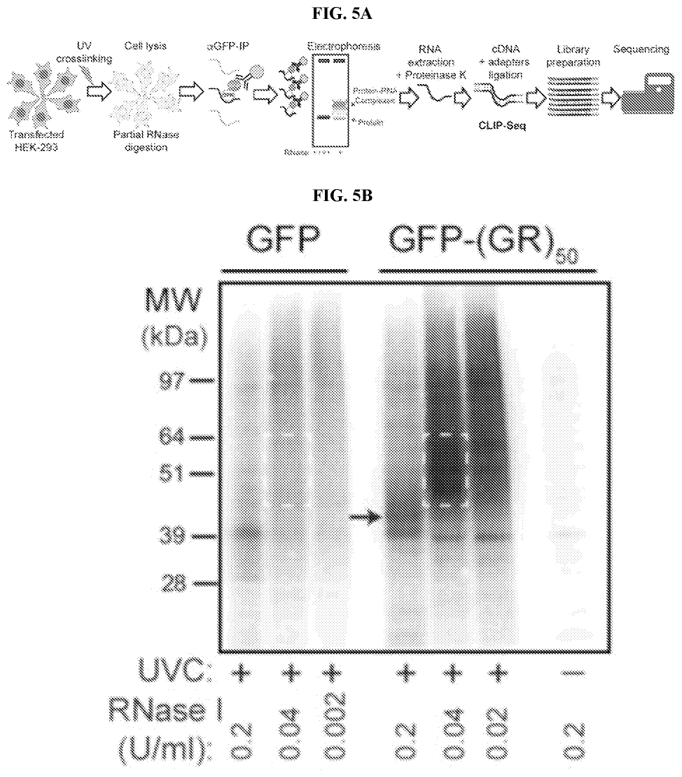

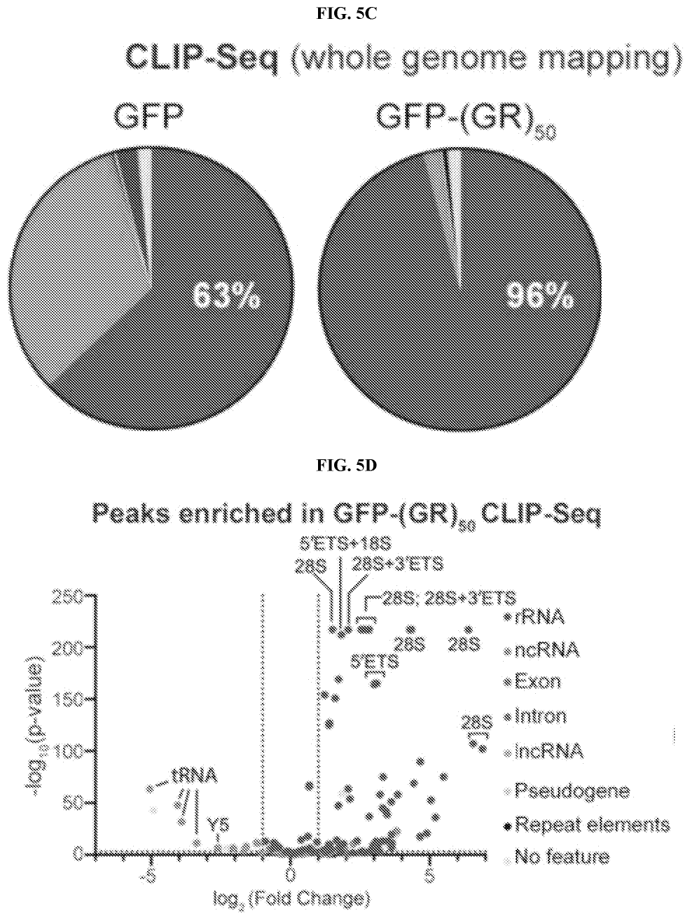

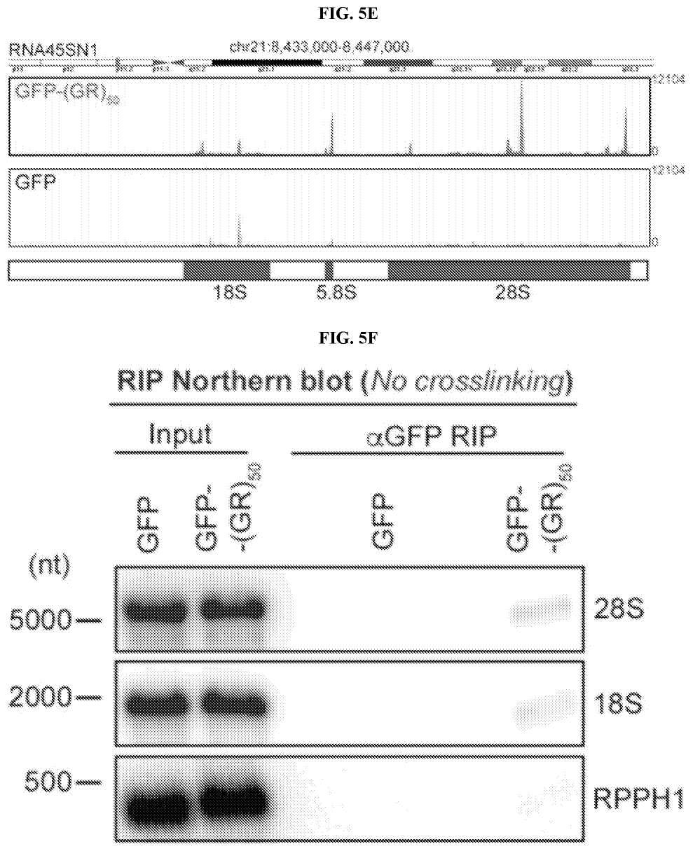

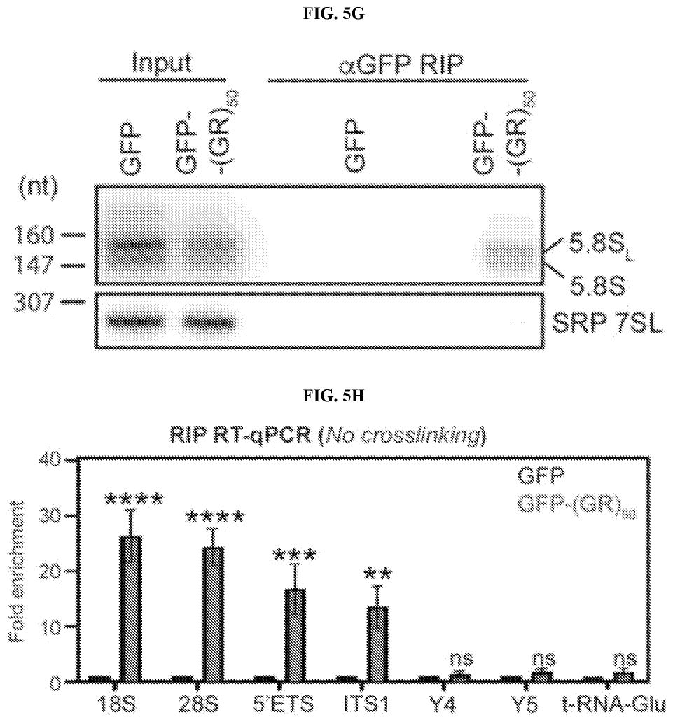

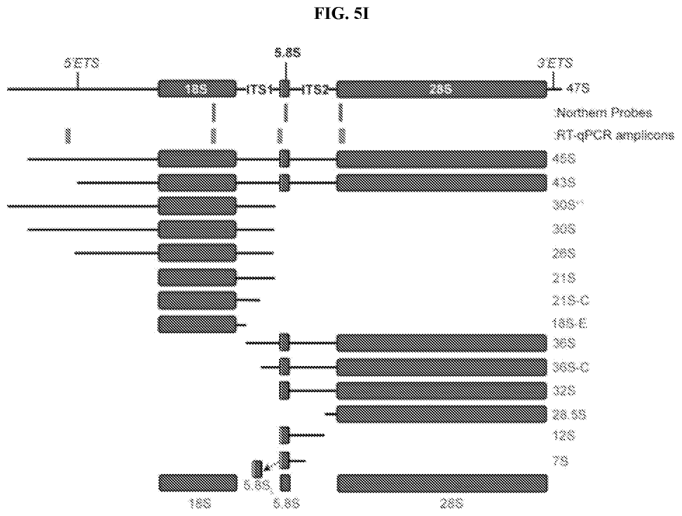

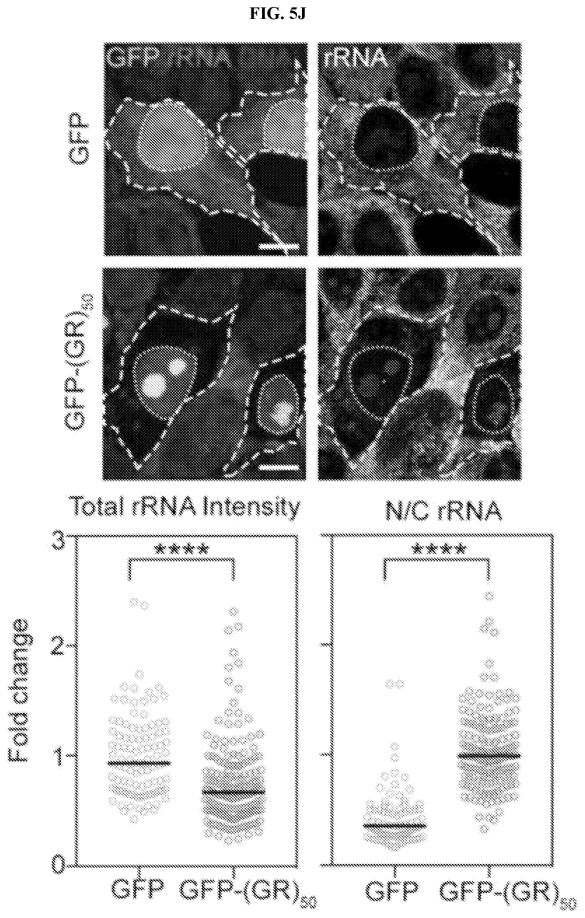

rRNA bait design. We design 20 nucleotide RNA baits based on the (GR)₅₀-interacting 28S rRNA sequence identified by CLIP-Seq (FIGS. 5A-5J). We introduced 2′-O-methylations in the five nucleotides at the 5′ and 3′ ends to improve their stability (mGmGmUmCmUrCrCrArArGrGrUrGrArAmCmAmGmCmC (SEQ ID NO: 15)). Using the same numbers of each of the 4 ribonucleotides we designed a scrambled sequence through the InvivoGen webtool www.invivogen.com/sirnawizard/scrambled.php (mGmCmAmCmUrGrArCrGrArUrGrCrGrCmUmAmGmCmA (SEQ ID NO: 46)). RNA baits were delivered with Lipofectamine RNAiMAX transfection Reagent in Opti-MEM medium (Invitrogen) for 24 hours following manufacturer instructions. To evaluate cellular internalization of the RNA baits, we added a Cy3™ fluorophore at the 3′ end of the ribonucleotide (mGmGmUmCmUrCrCrArArGrGrUrGrArAmCmAmGmCmC/3Cy3Sp/ (SEQ ID NO: 47)). All the RNA oligonucleotides utilized in this study were synthesized by Integrated DNA Technologies.

Quantitative image acquisition and analysis. Images used for quantification were acquired at matched exposure times or laser settings and processed using identical settings. Quantifications were normalized within each respective experiment with n≥3 independent experiments unless otherwise specified in figure legends. Image acquisition for HEK-293 experiments was performed on a Leica DMI4000B laser scanning confocal microscope (Leica, Buffalo Grove, IL) or with Leica DMi8 microscope (Leica, Buffalo Grove, IL) using a C10600-ORCA-R2 digital CCD camera (Hamamatsu Photonics, Japan), and processed with Fiji. For high-resolution images and 3D reconstructions Nikon W1 Dual CAM spinning Disk and Imaris Cell Imaging software were used.

Quantification and statistical analysis. All statistical analyses were done with Prism 7 software (GraphPad Software). Individual values were usually displayed by dots in the graphs, and represent all values measured in the study. The sample size (n) of each specific experiment are provided in the results section and the statistical test performed for each specific experiment is defined in the corresponding figure legend. For each statistical analysis, we first tested whether sample data fit into Gaussian distribution using the D'Agostino-Pearson omnibus normality test. To compare two experimental conditions, either a student's t test (parametric) or a Mann-Whitney U test (non-parametric) was performed. To compare ≥3 experimental conditions, either a One-Way or Two-Way ANOVA followed by a Bonferroni post-hoc test (parametric) or a Kruskal-Wallis rank test followed by a two-stage linear step-up procedure of Benjamini, Krieger and Yekutieli (non-parametric) was performed.

### Example 2: Characterization of the Physicochemical Features of C9 Arginine (R)-DPRs

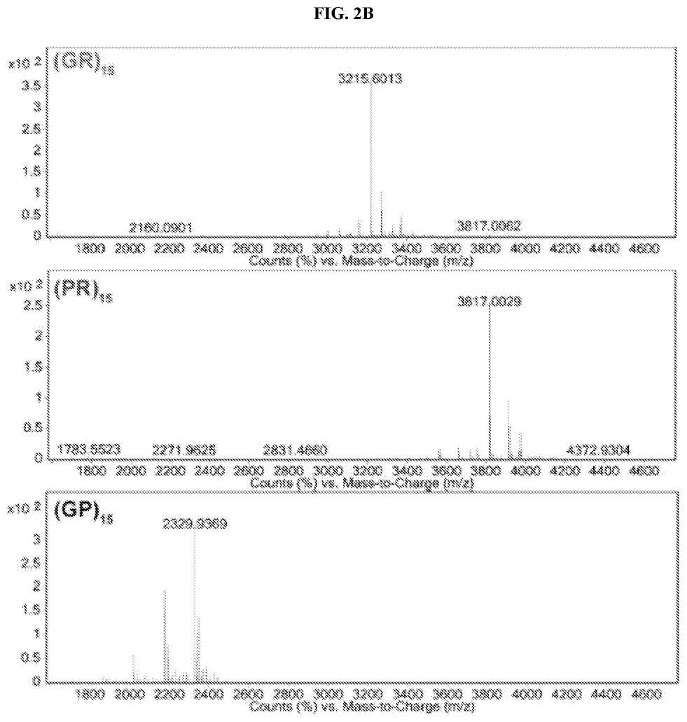

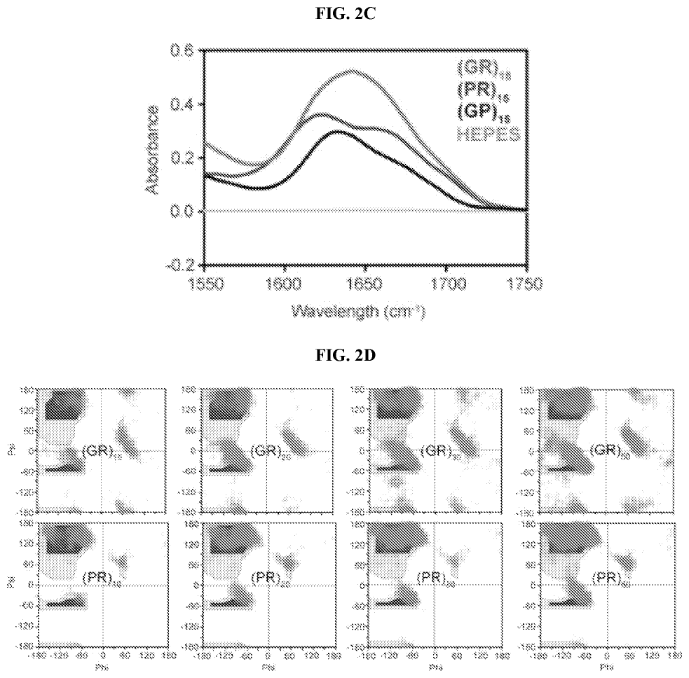

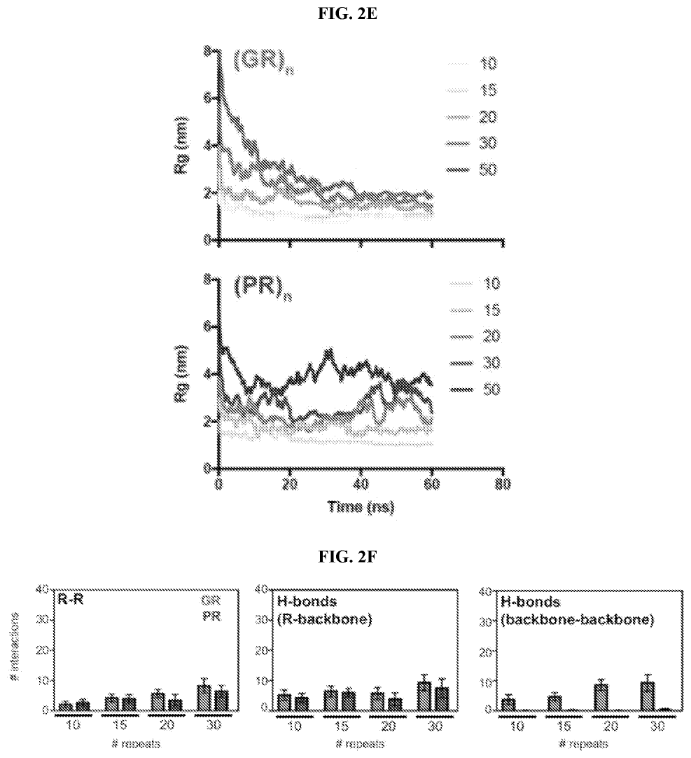

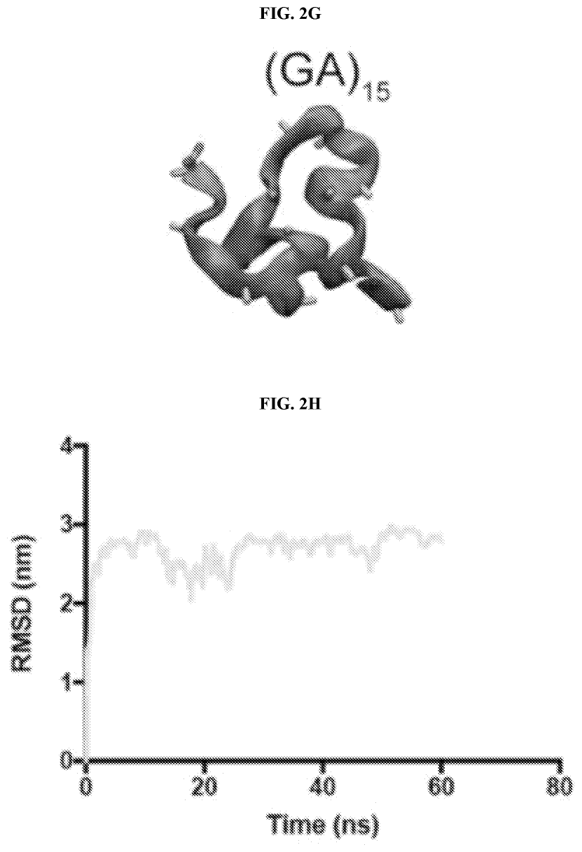

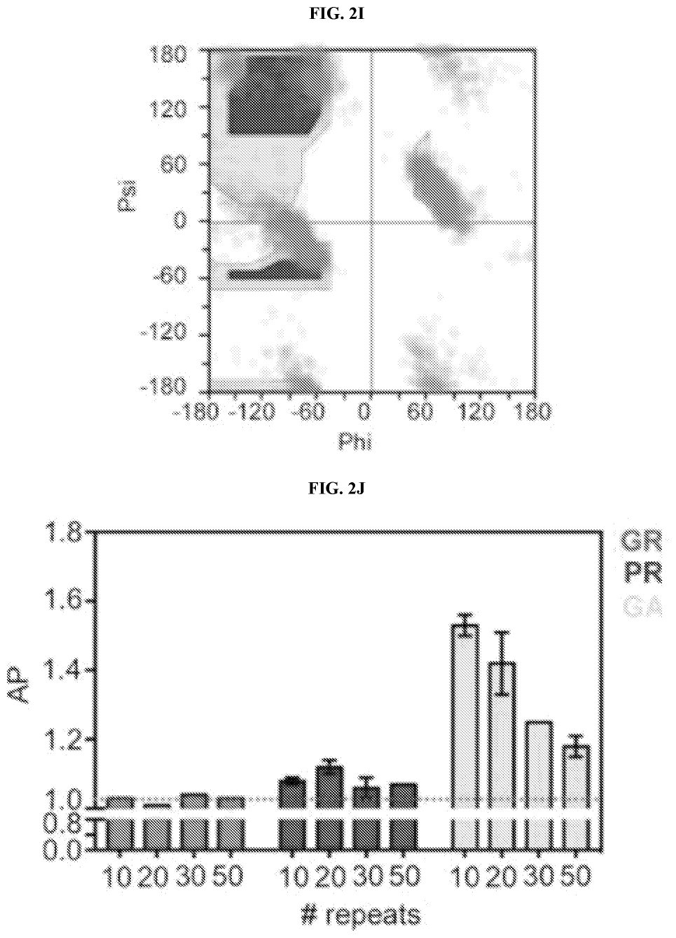

To interrogate the role of RNA in DPR pathophysiology we first characterized the structural and chemical features of (R)-DPRs. We specifically examined the secondary structure of the R-rich, highly toxic poly-GR and poly-PR by all atoms molecular dynamics (MD) simulations using CHARMM force field (FIGS. 1A-1B). As a control, we looked at the non-toxic poly-GP DPR. We modeled DPRs with 15 repeats, which represents the maximum length of peptides we could synthesize with high purity and corresponds to half of the minimum number of hexanucleotide (GGGGCC)n (SEQ ID NO: 48) repeats that is considered to be neurotoxic (n=30). While both (GP)₁₅ (SEQ ID NO: 10) and (GR)₁₅ (SEQ ID NO: 11) showed a highly coiled conformation, (PR)₁₅ (SEQ ID NO: 12) displayed a more stretched structure (FIGS. 1A-1B and 2A). The differential physicochemical properties of proline and glycine likely impact the differential conformation of DPRs with proline favoring a rigid backbone, and glycine conferring higher flexibility (Jafarinia et al., 2020). Along these lines, the calculation of torsional angles in the different DPR residues by Ramachandran plots showed that (PR)₁₅ (SEQ ID NO: 12) has a high enrichment in β-sheet conformations, while (GR)₁₅ (SEQ ID NO: 11), and (GP)₁₅ (SEQ ID NO: 10) displayed a more diverse profile of different secondary conformations in their structures (FIG. 1C). This correlates with a higher extended conformation observed in (PR)₁₅ (SEQ ID NO: 12), while (GP)₁₅ (SEQ ID NO: 10) and (GR)₁₅ (SEQ ID NO: 11) show more convoluted structures (FIG. 1B). To validate the results obtained by MD simulations we synthesized highly pure DPRs with 15 repeats (FIG. 2B) and characterized them by circular dichroism (CD). While (PR)₁₅ (SEQ ID NO: 12) showed β-sheet spectra, (GP)₁₅ (SEQ ID NO: 10) and (GR)₁₅ (SEQ ID NO: 11) displayed α-helix and random coiled spectra, respectively (FIG. 1D). Since solvent composition can impact CD analysis, we performed liquid and solid sampling of the C9 DPRs and performed Fourier-transform infrared spectroscopy (FTIR), which allows for collecting high-resolution spectral data. FTIR analysis of the peptides in aqueous solution showed that while all three exhibited a signal at 1675 cm-1, typical of rigid turns, this was strongly enhanced in R-DPRs (FIG. 1E). The stronger signal likely reflects the additional intramolecular R-R interactions that R-DPRs form as shown in the MD simulations. The signals at 1585 and 1610 cm⁻¹ for (GR)₁₅ (SEQ ID NO: 11) are caused by the guanidines of R side chains. The latter was enhanced and shifted to higher frequencies for (PR)₁₅ (SEQ ID NO: 12) (at around 1615 cm⁻¹, blue arrow), as it mixes with a low frequency amide I vibration, indicative of a β-sheet. The amide I was shifted to higher frequencies (red arrow) in the case of (GR)₁₅ (SEQ ID NO: 11), indicative of more coiled structures (FIG. 1E) (Barth, 2000; Barth and Zscherp, 2002). (GP)₁₅ (SEQ ID NO: 10) displayed a sum of amide I vibrations with similar intensity suggesting that it exhibits a combination of β-sheet, coil and turn conformations. In solid FTIR analysis, the guanidine vibrations disappear, validating the β-sheet nature of the (PR)₁₅ (SEQ ID NO: 12) amide I vibration around 1620 cm⁻¹ (FIG. 2C). In contrast, (GR)₁₅ (SEQ ID NO: 11) and (GP)₁₅ (SEQ ID NO: 10) exhibited higher amide I frequencies, indicative of a coiled structure (FIG. 2C).

We next asked how the number of repeats would impact the structure of the DPRs using MD simulations. Ramachandran plots of both GR and PR with 10 (SEQ ID NOS 50 and 53, respectively), 20 (SEQ ID NOS 51 and 16, respectively), 30 (SEQ ID NOS 52 and 9, respectively) and 50 (SEQ ID NOS 17 and 18, respectively) repeats showed the same secondary structure patterns, irrespective of length (FIGS. 1F and 2D). Importantly, the higher flexibility of the poly-GR backbone favors a more folded conformation compared to the poly-PR backbone, which exhibited an extended conformation (FIGS. 1F and 2E). Accordingly, the higher level of folding observed in poly-GR peptides led to a size-dependent increase in the number of R-R and H-bond interactions within the peptide backbone relative to poly-PR (FIGS. 1G-1H and 2F). We also performed MD simulations to interrogate the intrinsic aggregation propensity (AP) of different size R-DPRs. As a positive control we used poly-GA, because of its well-described tendency to self-aggregate (Chang et al., 2016; Freibaum and Taylor, 2017; Jafarinia et al., 2020) (FIGS. 2G-2I). Although R-DPRs displayed similar AP profiles compared to poly-GA, values in poly-PR were slightly higher than poly-GR (FIG. 2J). Collectively, our computational and empirical analyses indicate that GR and PR exhibit differential secondary structures and aggregation propensities that appear to be only mildly affected by repeat length and could lead to differential pathophysiological effects.

### Example 3: Characterization of the Interaction Between C9 R-DPRs and RNA In Vitro

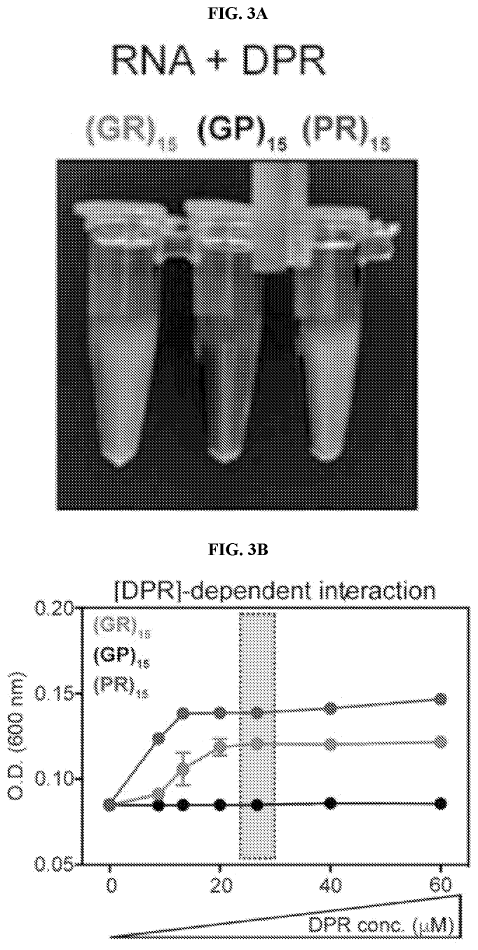

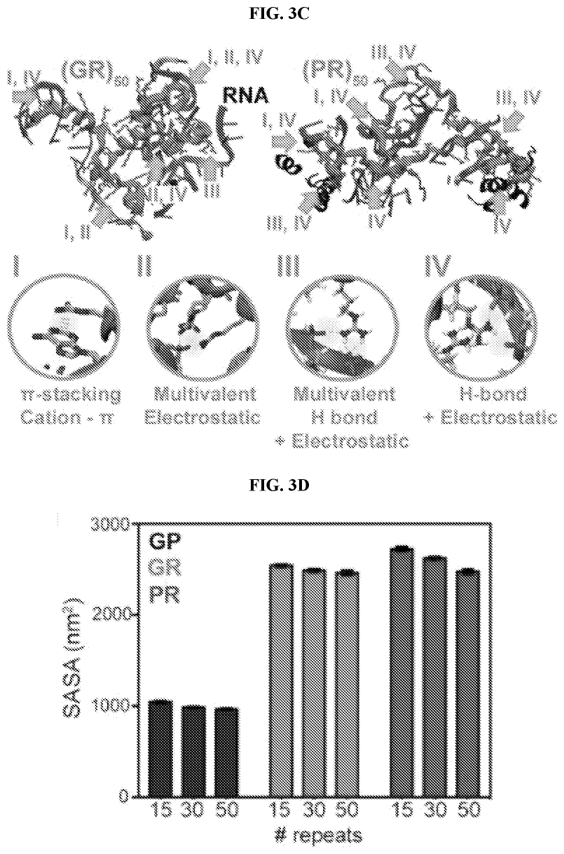

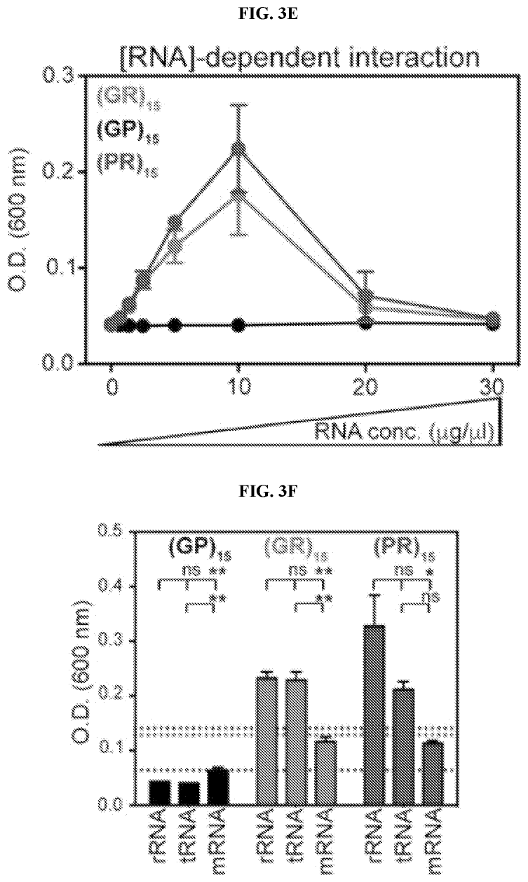

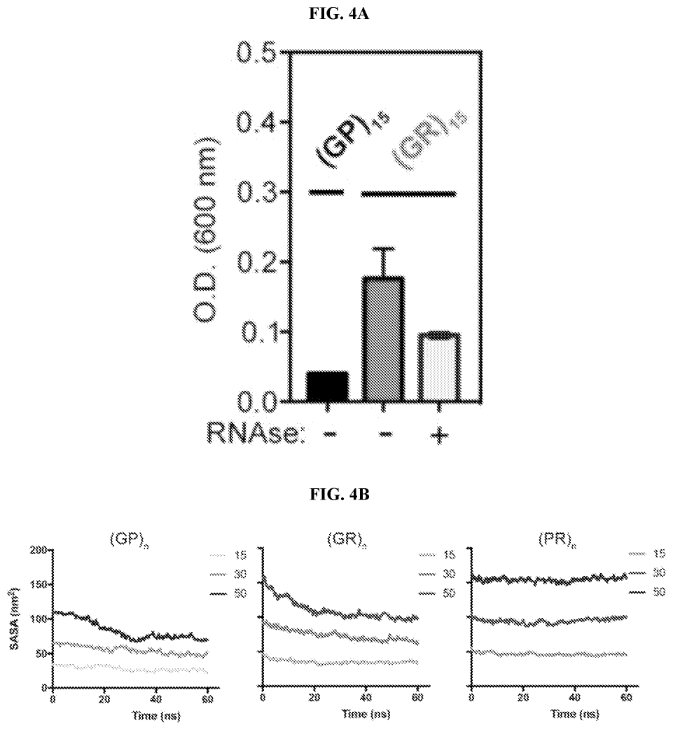

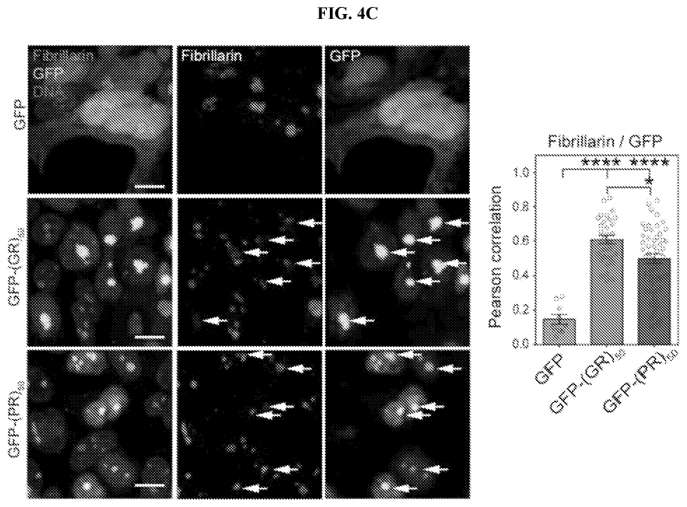

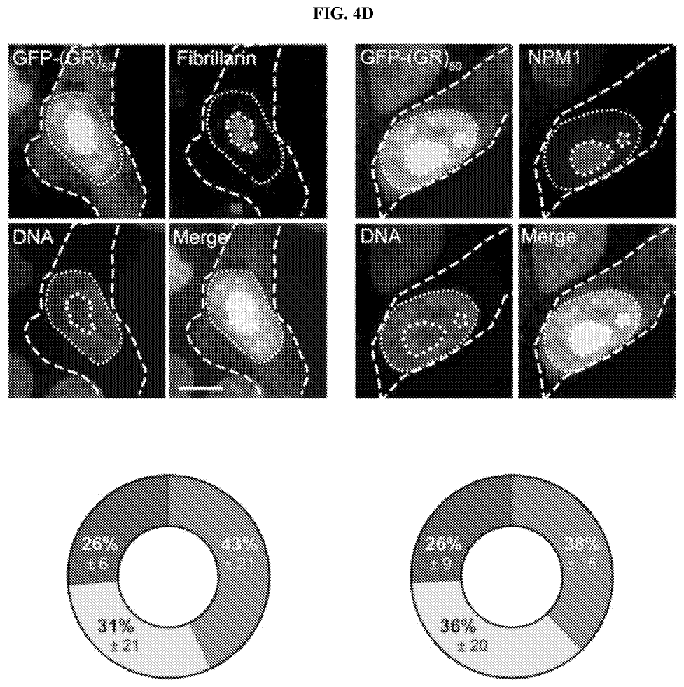

Previous studies have suggested that R-DPRs can bind to RNA molecules through ionic and cation-pi interactions (Boeynaems et al., 2017; Kanekura et al., 2016; White et al., 2019). Thus, we sought to assess whether the differential physicochemical properties of poly-GR and poly-PR DPRs would impact their binding to RNA molecules. We first incubated total human RNA with synthetic DPRs in vitro and measured optical density as an indicator of RNA-DPR interaction and aggregation (FIGS. 3A-3B, 4A). While (GP)₁₅ (SEQ ID NO: 10) had no effect, both R-DPRs aggregated with RNA in a concentration-dependent manner (FIGS. 3A-3B). A dose response experiment with increasing concentrations of DPRs showed that the interaction of (GR)₁₅ (SEQ ID NO: 11) with RNA peaked at 20 μM, while (PR)₁₅ (SEQ ID NO: 12) peaked at 15 μM, suggesting a stronger potential for PR to bind and precipitate RNA (FIG. 3B). This is in accordance with its expanded structural conformation (FIGS. 1A-1H and 2A-2J) and by extension, the higher number of cations available for multiple electrostatic or cation-pi interactions with RNAs (FIG. 3C). Indeed, calculation of the solvent-accessible surface area (SASA) of both R-DPRs and poly-GP indicated higher values in poly-(PR) than the other DPRs, particularly for the 15 and 30 repeat dipeptides (FIGS. 3D and 4B). We next interrogated how the concentration of RNA would impact the propensity for R-DPRs to aggregate. We observed that while the gradual increase in RNA concentration of up to 10 μg/l increased the OD values, higher concentrations reduced aggregation (FIG. 3E), suggesting that the interaction is both RNA and R-DPR concentration-dependent. Finally, we asked whether the R-DPRs differentially interact with distinct types of RNA, including ribosomal (rRNA), transfer (tRNA) and messenger RNA (mRNA) in vitro. We found that both R-DPRs interacted more strongly with rRNA than mRNA (FIG. 3F), correlating with the fact that in cells they accumulate in nucleoli (FIGS. 4C-4D). Moreover, (GR)₁₅ (SEQ ID NO: 11) exhibited a stronger interaction with tRNA than mRNA (FIG. 3F). Collectively, our findings are in line with previous studies in demonstrating that poly R-DPRs bind RNA molecules in vitro (Boeynaems et al., 2017; Kanekura et al., 2016), and also suggest that R-DPRs may preferentially recognize structured RNAs, such as tRNA and rRNA.

### Example 4: Poly-GR Binds to Ribosomal RNA In Vivo

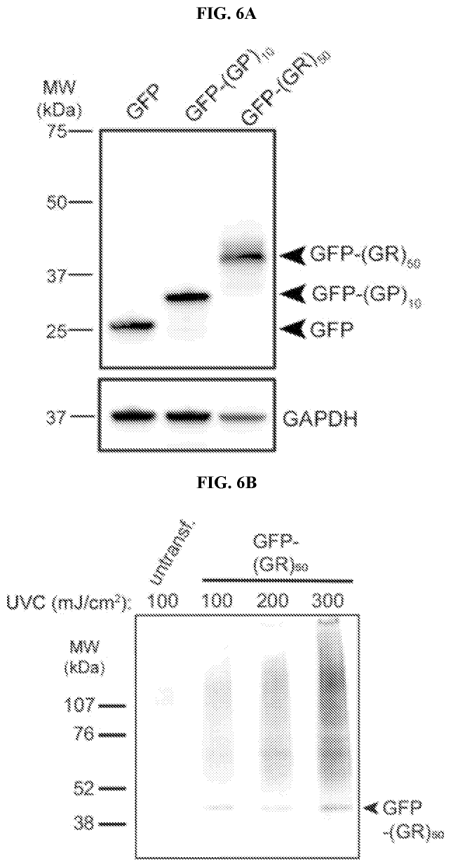

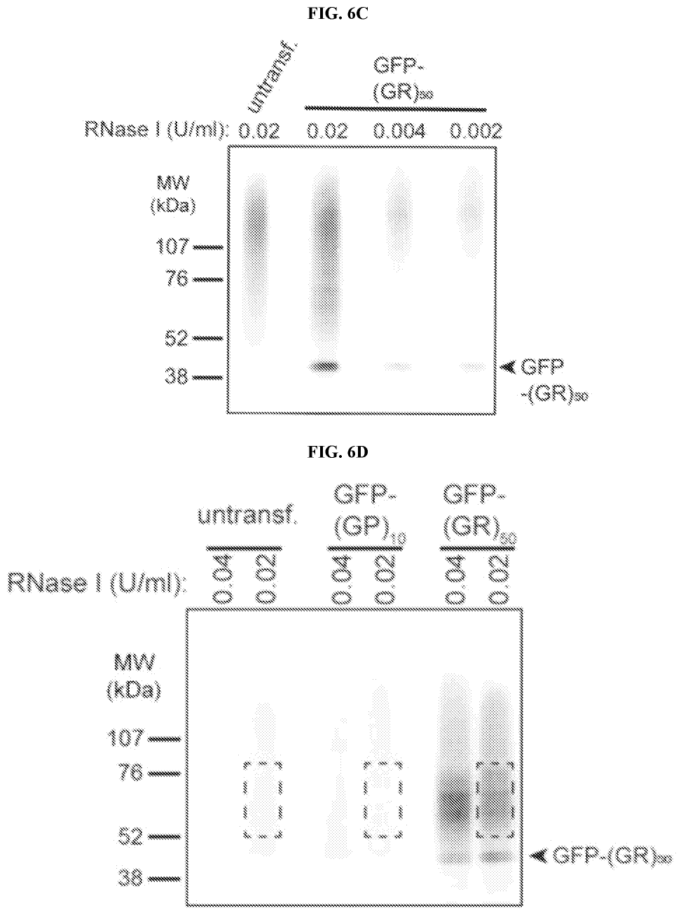

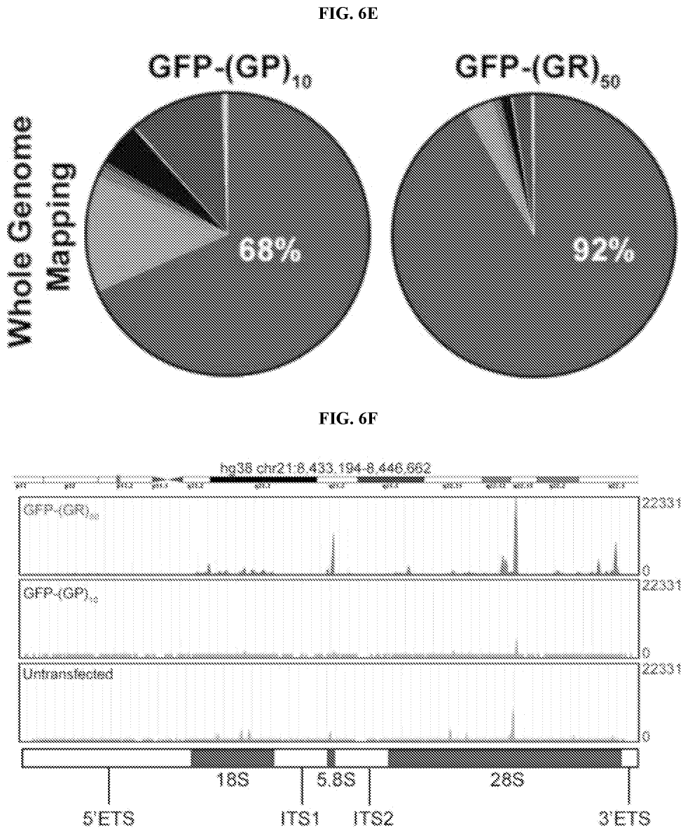

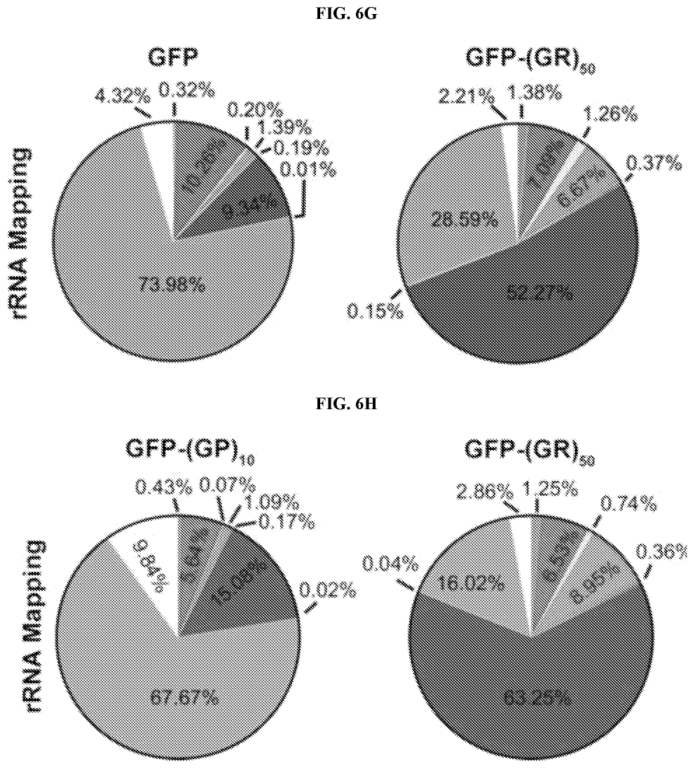

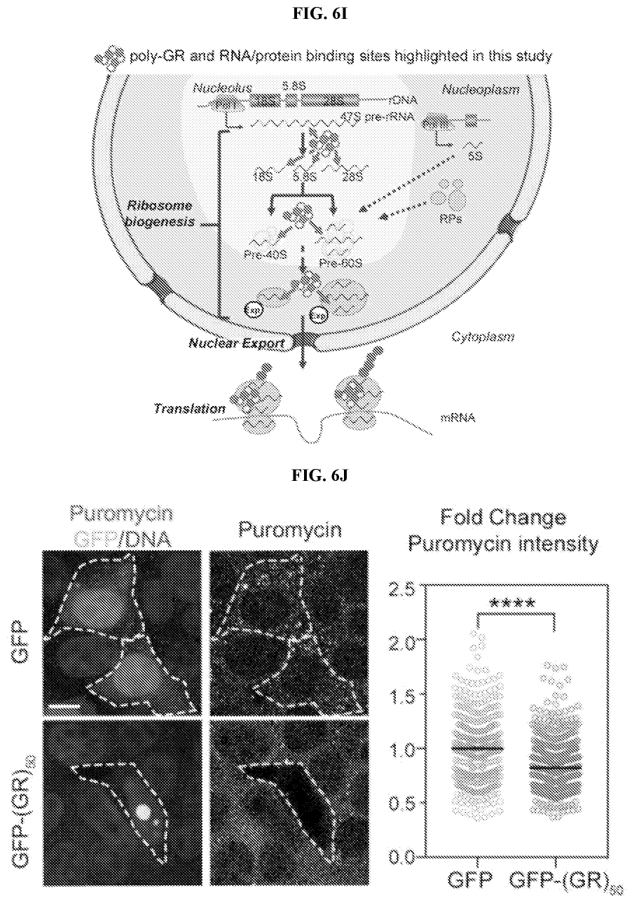

We next sought to identify the RNAs bound by poly-GR in vivo. We focused our analysis on poly-GR since it is more abundant than poly-PR in C9-ALS/FTD patient tissue (Gendron et al., 2013; Gomez-Deza et al., 2015; Mackenzie et al., 2015; Mori et al., 2013), and its abundance has been associated with affected brain areas in patients (Saberi et al., 2018). We used cross-linking immunoprecipitation followed by high-throughput sequencing (CLIP-Seq) (Darnell et al., 2018) to identify targets of poly-GR on a transcriptome-wide scale. In CLIP-Seq, UV light is used to cross-link proteins to RNAs that are in direct contact in vivo. After immunoprecipitating the protein of interest and harsh washing to remove noncovalently associated RNA, cDNA is prepared from the cross-linked RNA and sequenced (FIG. 5A). In preliminary experiments, we confirmed that, following transfection into HEK-293 cells, GFP-(GR)₅₀ could be crosslinked to RNA (FIGS. 6A-6C). To identify specific targets, we transfected HEK-293 cells with GFP-(GR)₅₀, using GFP (FIG. 5B) or GFP-(GP)₁₀ and untransfected cells (FIG. 6D) as controls. Following UV-crosslinking, immunoprecipitation and cDNA sequencing, we found that GFP-(GR)₅₀ interacted predominantly with rRNA. In two biological replicates, 96% and 92% of the GFP-(GR)₅₀ CLIP peaks, defined as five or more overlapping sequencing reads at a specific genomic locus, mapped to ribosomal DNA (rDNA) (FIGS. 5C and 6E). Additionally, while 63% and 68% of the CLIP peaks from the control samples also mapped to rDNA, the rRNA-derived peaks were strongly enriched in GFP-(GR)₅₀, while tRNAs and Y RNAs were de-enriched in GFP-(GR)₅₀ (FIG. 5D). Close inspection of the RNA45SN1 locus revealed that the GFP-(GR)₅₀-derived peaks were not distributed evenly across the rDNA, but rather occurred in specific regions (FIGS. 5E and 6F).

Human rDNA exists as hundreds of copies of tandem repeats on five chromosomes (Potapova and Gerton, 2019). Although these repeats are not included in the assembled hg38 genome, five complete rDNA sequences that exist outside these clusters are included (RNA45SN1-5). Additionally, the genome is littered with divergent and truncated rRNA sequences that do not code for bonafide rRNA (Lander et al., 2001). Thus, to simplify quantification of the rRNA-derived reads, we remapped data from the CLIP experiments to a single full-length rDNA sequence, the RNA45SN1 locus (NCBI Accession NR 146117.1). We found that 69% and 81% of the total reads aligned to rDNA for the GFP-(GR)₅₀ libraries (FIGS. 6G-611). In contrast, only 22% of the reads in the GFP control and 23% of the GFP-(GP)₁₀ control libraries mapped to rDNA (FIGS. 6G-611). Interestingly, the overwhelming majority of GFP-(GR)₅₀ reads were derived from the 28S region of the rDNA, with 52% and 63% of the total reads in the two libraries aligning to this region. Within the 28S rRNA, a prominent peak was located between nucleotides 2744 and 2810. This 66 nucleotide region was the most highly enriched site in both CLIP experiments accounting for 30% and 45% of total reads in the GFP-(GR)₅₀ samples. An additional 57-nucleotide peak at the 3′ end of the 5.8S rRNA accounted for 6% and 9% of the reads in the two GFP-(GR)₅₀ datasets.

Since the production of mature rRNA involves a complex series of processing steps that occur almost entirely within nuclei, the GFP-(GR)₅₀ CLIP peaks that we detected mapping to the 28S and 5.8S sites in the rDNA could be derived from nuclear precursors such as 47S, 45S, 30S, or 32S, and/or the mature cytoplasmic forms (5.8S, 28S) (FIG. 5I). To distinguish precursors from mature rRNAs, we used ribonucleoprotein immunoprecipitation (RIP), followed by Northern blot (NB) analysis, to further characterize the bound rRNAs. These experiments revealed that mature 28S, 18S and 5.8S rRNAs were all detected in GFP-(GR)₅₀, but not in GFP immunoprecipitates, as would be expected if mature cytoplasmic ribosomes were targets (FIGS. 5F-5G). Consistent with specific binding, two other abundant RNAs, the SRP 7SL RNA and the RNase P RNA RPPH1, were not detected in these immunoprecipitates. While these data demonstrate an association between GFP-(GR)₅₀ and mature ribosomes, some CLIP reads could only have originated from immature rRNA (FIGS. 6G-6I). The 5′ ETS and ITS1 regions were the most abundant precursor regions detected, each accounting for 0.7 to 1.3% of the reads in the GFP-(GR)₅₀ CLIP datasets. Because these pre-rRNAs were not abundant enough to detect by NB, we assayed for their presence in our RIP experiment using RT-qPCR. Consistent with our NB results, RT-qPCR showed strong enrichment of mature 28S and 18S rRNAs in the GFP(GR)₅₀ immunoprecipitates. This assay also demonstrated enrichment of sequences from the 5′ ETS and ITS1 regions of the pre-rRNA (FIG. 5H). Noncoding Y RNAs and tRNA-Glu were selected as controls since these RNAs were not enriched in the GFP-(GR)₅₀ CLIP-Seq dataset (FIG. 5D). As expected, these RNAs showed no significant enrichment in the GFP-(GR)₅₀ RIP (FIG. 5H).

Collectively, these results demonstrate that poly-GR binds to multiple rRNA species, including precursors found in the nucleolus and mature RNAs found in both the nucleolus and within fully assembled cytoplasmic ribosomes (FIG. 6I). These findings are well-aligned with the subcellular localization of GR, a proportion of which accumulates within both the nucleolus and cytoplasm (FIG. 4D). These interactions could potentially impact ribosomal assembly, homeostasis and/or function. In line with previous studies (Lee et al., 2016; White et al., 2019), immunolabeling to detect rRNA revealed reduced levels of total rRNA in cells transfected with poly-GR (FIG. 5J). Importantly, we found a significant shift in the nucleocytoplasmic (N/C) ratio of rRNA signal in cells transfected with GFP-(GR)₅₀ (FIG. 5J). This increased N/C ratio in vivo, which suggests impaired ribosomal homeostasis, likely contributes to the previously described reduced level of translation in poly-GR-containing cells (FIG. 6J) (Hartmann et al., 2018; Lee et al., 2016; Zhang et al., 2018b).

### Example 5: The Interaction of Poly-GR with rRNA Strongly Contributes to Ribosomal Binding

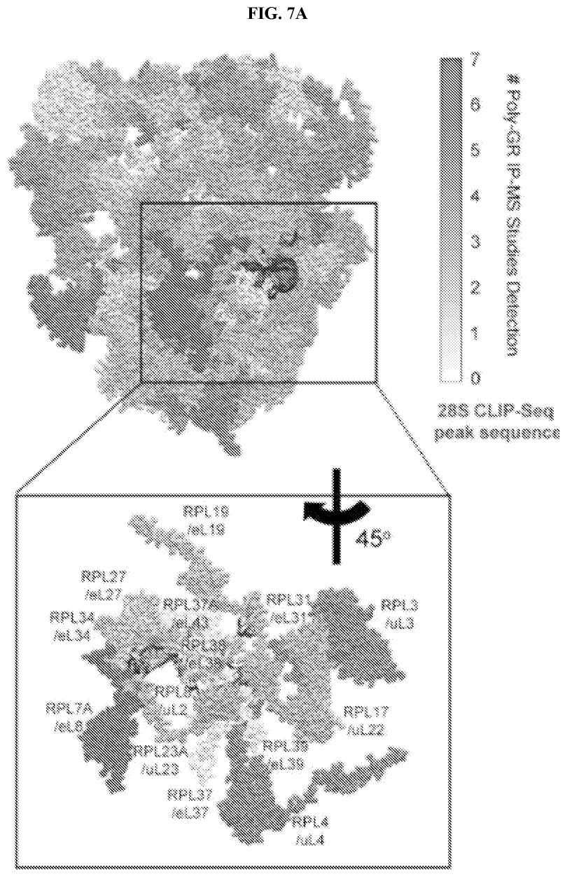

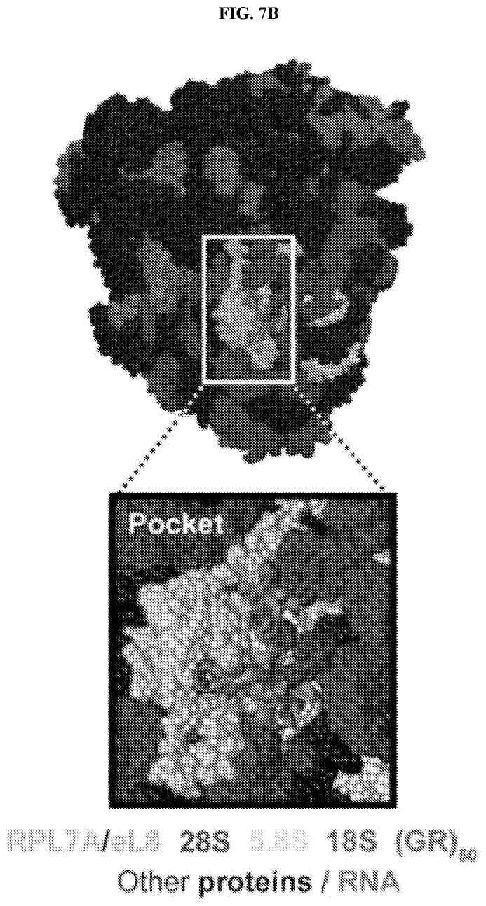

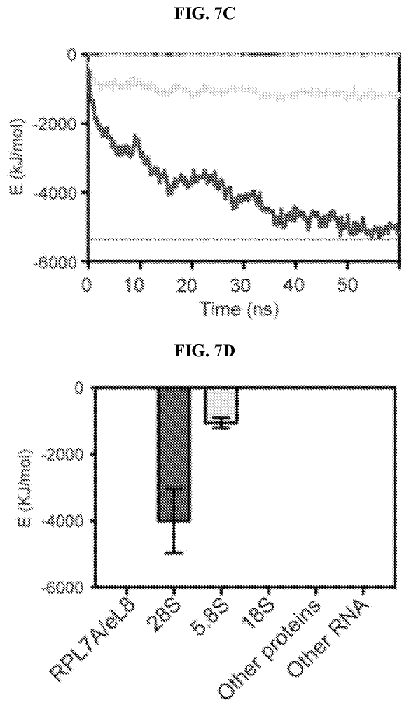

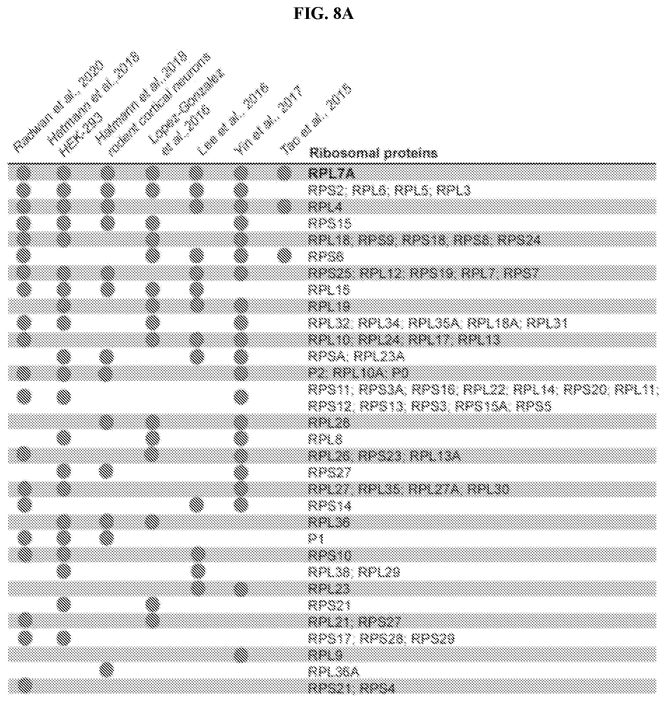

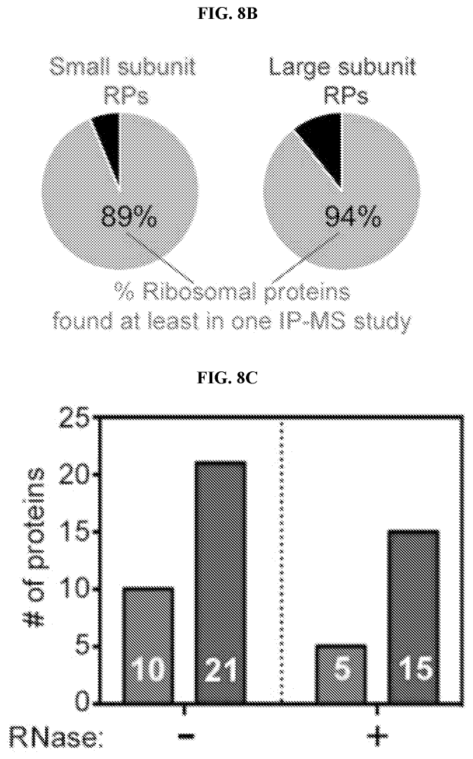

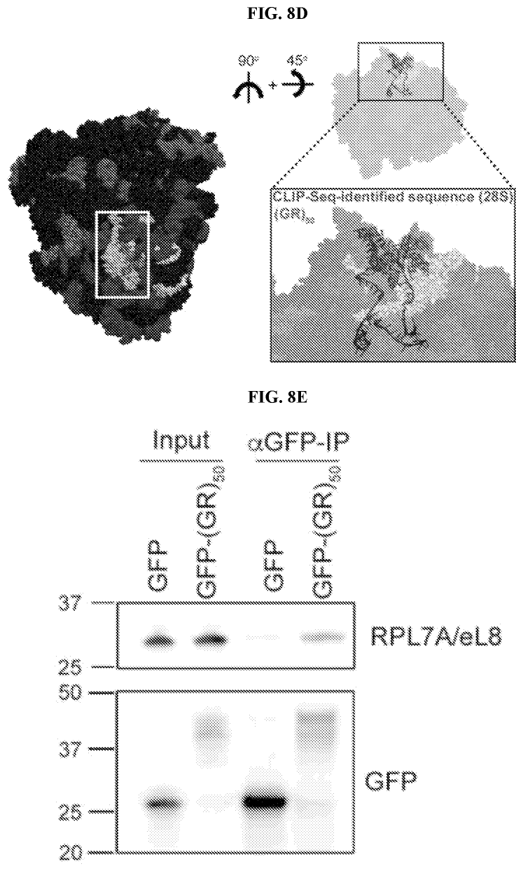

The identification of rRNA as a binding partner for poly-GR in vivo is well aligned with previous mass spectrometry (MS)-based studies, that have consistently uncovered ribosomal proteins as GR interactors after immunoprecipitation (FIG. 8A) (Hartmann et al., 2018; Lee et al., 2016; Lopez-Gonzalez et al., 2016; Radwan et al., 2020; Tao et al., 2015; Yin et al., 2017). In fact, 89% and 94% of all ribosomal proteins of the small and large ribosomal subunits respectively, have been reported to precipitate along with poly-GR in at least one of the previously reported interactomes (FIGS. 8A-8B). Intriguingly, one study showed that more than 40% of interactions with ribosomal protein subunits disappear after RNA degradation by RNase treatment, suggesting that rRNA might be playing a critical role in the physical association of the ribosome with C9 poly-GR DPR (FIG. 8C) (Lopez-Gonzalez et al., 2016). In order to more directly investigate the significance of the GR-rRNA interaction in vivo, we sought to model the poly-GR-ribosome complex. We used cryo-EM-based structural information of the mammalian ribosome (PDB ID: 5LZS; (Shao et al., 2016), and observed that some regions of the 5.8S and 28S rRNA, which were crosslinked to poly-GR in vivo, are exposed to the surface of the ribosome (FIGS. 7A-7B). Interestingly, one of the closest ribosomal subunit proteins within this region is RPL7A/eL8, which is the only subunit that has been identified in all seven published GR interactome experiments, and one that we validated in our model system (FIGS. 7A and 8A, 8D-8E) (Hartmann et al., 2018; Lee et al., 2016; Lopez-Gonzalez et al., 2016; Radwan et al., 2020; Tao et al., 2015; Yin et al., 2017).

Based on this converging evidence from proteomic and transcriptomic experiments we established an in-silico model and mapped one, of likely many regions that poly-GR can bind to, on the surface of the ribosome (FIGS. 7B and 9A-9L). This potential binding “pocket” within the modelled poly-GR-ribosome assembly, accommodates the physicochemical interaction of poly-GR with both rRNA and ribosomal proteins (RPs). To better understand the dynamics of this interaction we performed molecular dynamics (MD) simulations. We specifically asked how strongly the different ribosomal components that are aligned either within the pocket (RPL7A/eL8, 5.8S, 28S and 18S), or in close proximity to the pocket (other proteins and RNA), contribute to the binding with (GR)₅₀ (SEQ ID NO: 17) by comparing their energy of interaction (E<0 attractive and E>0 repulsive) (FIGS. 7B-7D). Intriguingly, this analysis showed that the 28S rRNA species had the highest energy of interaction with (GR)₅₀ (SEQ ID NO: 17) (E=−4004.79 kJ/mol), followed by 5.8S (E=−1059.01 KJ/mol), while RPL7A/eL8 and 18S exhibited negligent E values (FIGS. 7C-7D). We also simulated the interaction of (GR)₅₀ (SEQ ID NO: 17) in a different ribosomal region, where there are RPs that have never been immunoprecipitated with poly-GR (no GR-RPs interaction control region) (FIGS. 9D-9I). We found that (GR)₅₀ (SEQ ID NO: 17) interacted exclusively with 18S rRNA in this region, although with weaker energy of interaction relative to the one exhibited for 28S rRNA in the pocket region (FIGS. 7C and 9I). Of note, (GR)₅₀ (SEQ ID NO: 17) acquires a more folded configuration within the pocket than within the control region (FIGS. 9J-9K), suggesting a higher level of conformational adaptation in the pocket, likely due to its stronger interaction with the 28S rRNA. Thus, our computational and CLIP-Seq data (FIGS. 5A-5J and 7A-7D) suggest that interactions between poly-GR and rRNA contribute to ribosomal binding.

### Example 6: Custom rRNA Oligonucleotides Bind Poly-GR and Inhibit the Toxic Effects of Poly-GR on Ribosomal Homeostasis

Collectively, our data indicate that the physicochemical properties of poly-GR promote strong interactions with RNA, while independent transcriptomic and proteomic analysis suggest that GR-DPR can interact with multiple ribosomal RNA species and protein subunits (FIGS. 5A-5J, 7A-7D and 8A-8E, 9A-9L). Importantly, these interactions can take place with both assembling ribosomal subunits in the nucleus and mature cytoplasmic ribosomes, and likely contribute to an impairment in ribosomal homeostasis and function (FIGS. 5J and 6I-6J). Moreover, our dynamic simulation models suggest that the strong rRNA-GR binding may mediate the interaction between the R-DPR and the ribosome. We thus reasoned that an RNA molecule of the right sequence and structure could act as a “bait” for poly-GR, binding to it and sequestering it away from pathological interactions with other proteins.

To test this hypothesis, we used the 28S rRNA sequence that was highly enriched in our CLIP-Seq experiments and designed an oligonucleotide RNA bait with 2′-O-methyl modifications in both the 5′ and 3′ ends to enhance its stability and binding properties (FIG. 10A and Methods). We first confirmed that the bait could interact with synthetic GR₁₅ in vitro (FIG. 11A). We then transfected mammalian cells with fluorescent tagged-oligoribonucleotide bait and established that it was effectively internalized (FIG. 11B). We also observed that the RNA bait showed higher colocalization with GFP-(GR)₅₀ than with GFP, and accordingly, accumulated in the nucleus of (GR)₅₀ (SEQ ID NO: 17)-transfected cells at higher levels (FIG. 11C). Next, we delivered it to cells expressing GFP-(GR)₅₀ and monitored several metrics related to poly-GR and its adverse effects on ribosomal homeostasis. As a control, we used a scrambled RNA molecule of the same length and percentage of nucleotide composition. Critically, we did not observe any adverse toxic effects in cells treated with either the 28S rRNA-based or scrambled-RNA bait (FIG. 11D). The 28S rRNA bait resulted in a moderate but significant reduction in the N/C ratio of the GFP-GR signal relative to the untreated control, suggesting that binding to the bait affected the localization of GR (n=443 cells for no RNA bait; 572 cells for +Scr. bait; 491 cells for +28S rRNA bait; p<0.001) (FIG. 11E). As we described earlier, cells expressing GFP-(GR)₅₀ exhibited a significant reduction in total levels of rRNA and increased rRNA N/C ratio (n=157 cells for GFP; 213 cells for GFP-(GR)₅₀; p<0.0001), as well as a decrease in protein translation (n=178 cells for GFP; 153 cells for GFP-(GR)₅₀; p<0.0001) (FIGS. 10B-10E). Importantly, these deficits were significantly inhibited by the presence of the 28S-based bait, which restored rRNA levels and subcellular localization (n=186 cells; p<0.01 for total rRNA levels and p>0.99 for N/C rRNA ratio in comparison to GFP control; and p<0.0001 in comparison to untreated control), as well as protein translation, to levels that were equal to the ones seen in GFP-control cells (n=306 cells; p>0.99 in comparison to GFP control and p<0.0001 in comparison to untreated control) (FIGS. 10B-10E). Moreover, this protective effect was specific to the 28S bait, as its beneficial effects were significantly different relative to cells treated with the scrambled control molecule (n=269 cells; p<0.001 for both total rRNA levels and N/C rRNA ratio; n=303 cells; p<0.0001 for protein translation) (FIGS. 10B-10E). These experiments suggest that an rRNA bait molecule can effectively protect cells from the detrimental effects of poly-GR.

We next interrogated the effects of the 28S bait on a neuronal model of poly-GR toxicity. We differentiated healthy control iPSCs into spinal motor neurons (MNs) using a well characterized protocol (Ziller et al., 2018), and transduced the cultures with a lentivirus expressing GFP or GFP-(GR)₅₀. We used live cell imaging analysis to track individual neurons over the course of 90 days and found that poly-GR overexpressing MNs exhibited significantly reduced survival relative to GFP-expressing MNs (n=221 GFP MNs; and n=223 GFP-(GR)₅₀ MNs; 8-10% reduction, p=0.0038) (FIG. 13A-13I). The majority of degenerating neurons were characterized by the accumulation of poly-GR-GFP nuclear aggregates, and nuclear aggregation strongly predisposed MNs to degeneration (FIG. 13B-13C). Importantly, MNs with poly-GR-GFP nuclear aggregates progressively exhibited a significant reduction in total rRNA levels, accumulation of rRNA signal within the nucleus, and reduced protein translation (FIG. 13D). Treating these MN cultures with a single dose of 28S rRNA led to a significant increase in survival that was critically associated with a significant reduction in the percent of poly-GR-GFP expressing MNs with nuclear aggregates, and the MNs that have a 24% reduction of their mortality (FIG. 13E-13G). In contrast, the scrambled control had only a minor effect on survival and did not rescue the accumulation of nuclear poly-GR-aggregates. To assess if the 28S bait can successfully mitigate the toxic effects of the C9orf72 hexanucleotide repeat expansion (C9-HRE) in a more disease-relevant, physiological system, we used fibroblast-induced MNs (iMNs) (Shi et al., 2018), derived from C9-HRE ALS patients or healthy control subjects (FIG. 13H, top). Strikingly, administration of 28S rRNA significantly improved survival and reduced the hazard ratio in iMNs derived from three distinct C9 ALS patients, while the scrambled control RNA had no substantial effect (FIG. 13H-13I).

Lastly, to assess the ability of the rRNA bait molecule to ameliorate poly-GR toxicity in an intact nervous system in vivo, we utilized two Drosophila models of poly-GR overexpression (FIG. 12A and FIG. 14A-14C). Motor neuronal expression of (GR)₅₀-EGFP is lethal during development, with ˜99% of mutant flies dying at pupal stages and failing to eclose (n=161 flies; FIGS. 12A-12D). We reasoned that this highly toxic in vivo model system would represent a stringent platform to test for any beneficial effects of the modified 28S RNA molecule on GR-DPR toxicity. We first ensured that the administration of Cy3-conjugated bait in feeding media during early larval stages, lead to its sufficient uptake within larval brain cells (FIGS. 12A-12B). Importantly, we found that the 28S rRNA-based bait had no effect on control GFP flies, while it significantly mitigated GR toxicity in (GR)₅₀-EGFP mutant flies with up to 9.3% of animals effectively eclosing and reaching adult stages (FIG. 12D) (n=151-164 flies per group; p<0.001). We obtained similar results using an alternative fly model, were (GR)₃₆ (SEQ ID NO: 49) is specifically expressed in the fly eye. Treatment with 28S rRNA-based bait, had a moderate but highly significant effect, reducing the severity of (GR)₃₆ (SEQ ID NO: 49)-dependent eye degeneration and the appearance of necrotic patches (FIG. 14A-14C). Collectively, these experiments demonstrate that administration of a modified 28S rRNA bait molecule can significantly meliorate poly-GR-dependent toxicity in multiple model systems in vitro and in vivo.

### Example 7: Discussion

The discovery of the C9-HRE as the most prevalent genetic driver of ALS/FTD has stimulated intense interest in deciphering the pathophysiology associated with this mutation. Several studies have shown that C9-DPR proteins have detrimental effects in cellular systems and model organisms (Choi et al., 2019; Freibaum et al., 2015; Hao et al., 2019; Hartmann et al., 2018; Jovicic et al., 2015; Kwon et al., 2014; Lee et al., 2016; Mizielinska et al., 2014b; Tao et al., 2015; Wen et al., 2014; Zhang et al., 2018b; Zhang et al., 2019). We combined computational and experimental approaches to better understand how the interaction of R-DPR proteins with RNA contributes to their toxicity. We found that poly-GR directly binds to multiple rRNA species in cells and impedes ribosomal homeostasis. We showcased the strength of the poly-GR/rRNA interaction by using a custom rRNA-based oligonucleotide, which prevented the malignant effects of poly-GR on ribosomal localization and function. These findings reinforce the importance of ribosomal impairment in C9-ALS/FTD and highlight a novel approach for protecting against R-DPR pathological mechanisms.

The characterization of the physicochemical features of poly-GR and poly-PR underscored a number of similarities, as well as critical structural differences that likely define their localization, molecular interaction profile and toxic potential. Poly-GR acquires a random coiled conformation, while poly-PR is highly enriched in β-sheets because of the higher rigidity of prolines compared to glycines. This secondary configuration confers a more stretched conformation, allowing more pronounced exposure of positive charges and a distinct adaptability to interact with complex molecular geometries such as the ones that are required during phase separation (Boeynaems et al., 2017; Flores et al., 2016; Jafarinia et al., 2020; Kanekura et al., 2018; Lee et al., 2016). While the particular size of native DPR proteins produced in physiological models remains unknown, our analysis suggests that their structural features are principally maintained irrespective of repeat number. This finding supports the notion that C9-HRE toxicity is threshold dependent and does not strongly correlate with repeat size (Cammack et al., 2019; Gendron et al., 2017; Gijselinck et al., 2016; Suh et al., 2015; van Blitterswijk et al., 2013).

Our work strongly suggests that poly-GR compromises ribosomal homeostasis and impedes the ability of ribosomes to mediate protein translation. While the precise mechanism of translation inhibition remains unclear, we specifically observed that poly-GR affected the subcellular distribution of rRNA, leading to a high N/C ratio. We hypothesize that this effect is likely the result of poly-GR binding to multiple rRNA species found in both the nucleus and the cytoplasm. This finding is well-aligned with previously described defects in ribosomal biogenesis in the nucleus and protein translation in the cytosol (Hartmann et al., 2018; Kwon et al., 2014; Lee et al., 2016; Moens et al., 2019; Wen et al., 2014; White et al., 2019; Zhang et al., 2018a). Although this rRNA shift could also be attributed to a previously described interaction of R-DPRs with nuclear pore proteins (Jovicic et al., 2015; Lee et al., 2016; Shi et al., 2017; Zhang et al., 2018b), the effects of these interactions on nucleocytoplasmic transport of rRNA-protein complexes remain unclear (Hayes et al., 2020; Vanneste et al., 2019; Zhang et al., 2018a).

CLIP-Seq analysis revealed that rRNA was the major RNA target of poly-GR in cells. While the potential for R-DPRs to interact with negatively charged molecules such as RNA had been established (Boeynaems et al., 2017; Boeynaems et al., 2019; Jafarinia et al., 2020; Kanekura et al., 2016; White et al., 2019), the identity of interacting RNAs in vivo was not known. The fact that rRNA was the predominant target correlates with the localization of ˜40% of all poly-GR in the nucleolus (FIG. 4D). At the same time, our analyses suggest that poly-GR can bind to both immature RNA species that are found in the nucleolus, as well as fully processed rRNA found within cytosolic ribosomes. The predominant precipitation of multiple rRNA species was somewhat unexpected, since the physicochemical properties of poly-GR suggest that it should exhibit rather promiscuous affinity for RNA molecules. However, ribosomes are highly abundant in the cell and rRNA is topologically exposed all around the ribosomal surface along with RPs, a lot of which contain LCDs (Uversky, 2013). This would likely explain the recurrent precipitation of ribosomal proteins in multiple IP-MS studies (Hartmann et al., 2018; Lee et al., 2016; Lopez-Gonzalez et al., 2016; Radwan et al., 2020; Tao et al., 2015; Yin et al., 2017). Importantly, our dynamic simulations predicted that it is the RNA that mediates the binding of R-DPRs to the ribosome, at least in the specific region modelled. This region likely represents a physiological binding site as it accommodates both exposed rRNA and ribosomal protein subunits shown to bind to poly-GR (Hartmann et al., 2018; Lee et al., 2016; Lopez-Gonzalez et al., 2016; Radwan et al., 2020; Tao et al., 2015; Yin et al., 2017). However, it is likely that poly-GR interacts with ribosomes at multiple locations. In fact, a recent study based on cryo-EM analysis describes the accumulation of short synthetic R-(DPRs)₂₀ within the polypeptide tunnel of assembling ribosomes in vitro, and suggests that this accumulation perturbs protein translation (Loveland et al., 2020). Altogether, our results and the the different experimental approaches mentioned above, indicate that R-DPRs can bind to multiple ribosomal regions and at different maturation stages (FIG. 6I).

Research studies around the C9 mutation have highlighted the non-canonical translation of DPR proteins as a pathway that can be targeted therapeutically. While there are several efforts focused on identifying the molecular factors that specifically mediate the production of all DPR proteins (Cheng et al., 2018; Cheng et al., 2019; Green et al., 2017; Moens et al., 2019; Sonobe et al., 2018; Tabet et al., 2018; Westergard et al., 2019; Yamada et al., 2019), this may prove challenging. Alternatively, individual DPRs can be targeted by specific antibodies (Nguyen et al., 2020; Zhou et al., 2017), or as we propose here, by RNA oligonucleotides. The identification of a specific RNA target that natively interacts with poly-GR provided us with a unique opportunity to design a bait ribonucleotide molecule and assess its ability to protect cells by sequestering away poly-GR from its pathological interactions. Indeed, this molecule restored ribosomal homeostasis in (GR)₅₀-transfected cells. While we did not evaluate the potential of the bait to block poly-PR associated defects, our computational models suggest it likely will (FIGS. 10E-10K, 10L). This could be important, as accumulation of poly-PR has been additionally observed in spinocerebellar ataxia type 36 (SCA36) (McEachin et al., 2020), while therapeutically relevant C9 antisense oligonucleotides only target and degrade the sense transcript, and thus would not alleviate PR toxicity.

The mitigating effects of RNA molecules have been recently explored in the context of other ALS/FTD model systems. Specifically, total RNA has been shown to alter the phase transition of C9 R-DPRs (Boeynaems et al., 2017; Boeynaems et al., 2019), as well as to alleviate some of the pathophysiological mechanisms associated with R-DPR overexpression (Hayes et al., 2020). Moreover, RNA oligonucleotides of known TDP-43 target sequences can prevent inclusions and rescue mutant TDP-43 neurotoxicity (Mann et al., 2019). Although more work is required to understand how the binding of the 28S rRNA bait to poly-GR alleviates its pathophysiology, the promising results we present here support the notion that using bait RNAs is not only useful to study RNA-protein interactions (Jazurek et al., 2016), but also to protect neurons from the detrimental effects of mutant or aberrant proteins (Odeh and Shorter, 2020).

### REFERENCES

Abraham, M. J., Murtola, T., Schulz, R., Pill, S., Smith, J. C., Hess, B., and Lindahl, E. (2015). GROMACS: High performance molecular simulations through multi-level parallelism from laptops to supercomputers. SoftwareX 1-2, 19-25.
Babaian, A., Rothe, K., Girodat, D., Minia, I., Djondovic, S., Milek, M., Spencer Miko, S. E., Wieden, H. J., Landthaler, M., Morin, G. B., et al. (2020). Loss of m(1)acp(3)Ψ Ribosomal RNA Modification Is a Major Feature of Cancer. Cell Rep 31, 107611.
Balendra, R., and Isaacs, A. M. (2018). C9orf72-mediated ALS and FTD: multiple pathways to disease. Nat Rev Neurol 14, 544-558.
Barth, A. (2000). The infrared absorption of amino acid side chains. Progress in Biophysics and Molecular Biology 74, 141-173.
Barth, A., and Zscherp, C. (2002). What vibrations tell us about proteins. Q Rev Biophys 35, 369-430.
Berendsen, H. J. C., Postma, J. P. M., Gunsteren, W. F. v., DiNola, A., and Haak, J. R. (1984). Molecular dynamics with coupling to an external bath. The Journal of Chemical Physics 81, 3684-3690.
Best, R. B., Zhu, X., Shim, J., Lopes, P. E. M., Mittal, J., Feig, M., and MacKerell, A. D. (2012). Optimization of the Additive CHARMM All-Atom Protein Force Field Targeting Improved Sampling of the Backbone ϕ, ψ and Side-Chain χ1 and χ2 Dihedral Angles. Journal of Chemical Theory and Computation 8, 3257-3273.
Boeynaems, S., Bogaert, E., Kovacs, D., Konijnenberg, A., Timmerman, E., Volkov, A., Guharoy, M., De Decker, M., Jaspers, T., Ryan, V. H., et al. (2017). Phase Separation of C9orf72 Dipeptide Repeats Perturbs Stress Granule Dynamics. Mol Cell 65, 1044-1055 e1045.
Boeynaems, S., Holehouse, A. S., Weinhardt, V., Kovacs, D., Van Lindt, J., Larabell, C., Van Den Bosch, L., Das, R., Tompa, P. S., Pappu, R. V., et al. (2019). Spontaneous driving forces give rise to protein-RNA condensates with coexisting phases and complex material properties. Proc Natl Acad Sci USA 116, 7889-7898.
Brooks, B. R., Brooks III, C. L., Mackerell Jr., A. D., Nilsson, L., Petrella, R. J., Roux, B., Won, Y., Archontis, G., Bartels, C., Boresch, S., et al. (2009). CHARMM: The biomolecular simulation program. Journal of Computational Chemistry 30, 1545-1614.
Bussi, G., Donadio, D., and Parrinello, M. (2007). Canonical sampling through velocity rescaling. The Journal of Chemical Physics 126, 014101.
Cammack, A. J., Atassi, N., Hyman, T., van den Berg, L. H., Harms, M., Baloh, R. H., Brown, R. H., van Es, M. A., Veldink, J. H., de Vries, B. S., et al. (2019). Prospective natural history study of <em>C9orf72</em> ALS clinical characteristics and biomarkers. Neurology 93, e1605-e1617.
Chang, Y. J., Jeng, U. S., Chiang, Y. L., Hwang, I. S., and Chen, Y. R. (2016). The Glycine-Alanine Dipeptide Repeat from C9orf72 Hexanucleotide Expansions Forms Toxic Amyloids Possessing Cell-to-Cell Transmission Properties. J Biol Chem 291, 4903-4911.
Cheng, W., Wang, S., Mestre, A. A., Fu, C., Makarem, A., Xian, F., Hayes, L. R., Lopez-Gonzalez, R., Drenner, K., Jiang, J., et al. (2018). C9ORF72 GGGGCC repeat-associated non-AUG translation is upregulated by stress through eIF2α phosphorylation. Nature Communications 9, 51.
Cheng, W., Wang, S., Zhang, Z., Morgens, D. W., Hayes, L. R., Lee, S., Portz, B., Xie, Y., Nguyen, B. V., Haney, M. S., et al. (2019). CRISPR-Cas9 Screens Identify the RNA Helicase DDX3X as a Repressor of C9ORF72 (GGGGCC)ₙ Repeat-Associated Non-AUG Translation. Neuron 104, 885-898.e888.
Choi, S. Y., Lopez-Gonzalez, R., Krishnan, G., Phillips, H. L., Li, A. N., Seeley, W. W., Yao, W. D., Almeida, S., and Gao, F. B. (2019). C9ORF72-ALS/FTD-associated poly(GR) binds Atp5a1 and compromises mitochondrial function in vivo. Nature neuroscience 22, 851-862.
Church, G. M., and Gilbert, W. (1984). Genomic sequencing. Proceedings of the National Academy of Sciences 81, 1991-1995.
Cook, N. J., Li, W., Berta, D., Badaoui, M., Ballandras-Colas, A., Nans, A., Kotecha, A., Rosta, E., Engelman, A. N., and Cherepanov, P. (2020). Structural basis of second-generation HIV integrase inhibitor action and viral resistance. Science 367, 806-810.
Darden, T., York, D., and Pedersen, L. (1993). Particle mesh Ewald: An N-log(N) method for Ewald sums in large systems. The Journal of Chemical Physics 98, 10089-10092.
Darnell, J. C., Mele, A., Hung, K. Y. S., and Darnell, R. B. (2018). Immunoprecipitation and SDS-PAGE for Cross-Linking Immunoprecipitation (CLIP). Cold Spring Harb Protoc 2018.
de Jong, D. H., Baoukina, S., Ingólfsson, H. I., and Marrink, S. J. (2016). Martini straight: Boosting performance using a shorter cutoff and GPUs. Computer Physics Communications 199, 1-7.
de Jong, D. H., Singh, G., Bennett, W. F. D., Arnarez, C., Wassenaar, T. A., Schafer, L. V., Periole, X., Tieleman, D. P., and Marrink, S. J. (2013). Improved Parameters for the Martini Coarse-Grained Protein Force Field. Journal of Chemical Theory and Computation 9, 687-697.
DeJesus-Hernandez, M., Mackenzie, I. R., Boeve, B. F., Boxer, A. L., Baker, M., Rutherford, N. J., Nicholson, A. M., Finch, N. A., Flynn, H., Adamson, J., et al. (2011). Expanded GGGGCC hexanucleotide repeat in noncoding region of C9ORF72 causes chromosome 9p-linked FTD and ALS. Neuron 72, 245-256.
Denning, E. J., Priyakumar, U. D., Nilsson, L., and Mackerell Jr., A. D. (2011). Impact of 2′-hydroxyl sampling on the conformational properties of RNA: Update of the CHARMM all-atom additive force field for RNA. Journal of Computational Chemistry 32, 1929-1943.
Donnelly, C. J., Zhang, P. W., Pham, J. T., Haeusler, A. R., Mistry, N. A., Vidensky, S., Daley, E. L., Poth, E. M., Hoover, B., Fines, D. M., et al. (2013). RNA toxicity from the ALS/FTD C9ORF72 expansion is mitigated by antisense intervention. Neuron 80, 415-428.
Flores, B. N., Dulchavsky, M. E., Krans, A., Sawaya, M. R., Paulson, H. L., Todd, P. K., Barmada, S. J., and Ivanova, M. I. (2016). Distinct C9orf72-Associated Dipeptide Repeat Structures Correlate with Neuronal Toxicity. PLoS One 11, e0165084.
Frederix, P. W., Scott, G. G., Abul-Haija, Y. M., Kalafatovic, D., Pappas, C. G., Javid, N., Hunt, N. T., Ulijn, R. V., and Tuttle, T. (2015). Exploring the sequence space for (tri-)peptide self-assembly to design and discover new hydrogels. Nat Chem 7, 30-37.
Frederix, P. W. J. M., Ulijn, R. V., Hunt, N. T., and Tuttle, T. (2011). Virtual Screening for Dipeptide Aggregation: Toward Predictive Tools for Peptide Self-Assembly. The Journal of Physical Chemistry Letters 2, 2380-2384.
Freibaum, B. D., Lu, Y., Lopez-Gonzalez, R., Kim, N. C., Almeida, S., Lee, K. H., Badders, N., Valentine, M., Miller, B. L., Wong, P. C., et al. (2015). GGGGCC repeat expansion in C9orf72 compromises nucleocytoplasmic transport. Nature 525, 129-133.
Freibaum, B. D., and Taylor, J. P. (2017). The Role of Dipeptide Repeats in C9ORF72-Related ALS-FTD. Front Mol Neurosci 10, 35.
Gendron, T. F., Bieniek, K. F., Zhang, Y. J., Jansen-West, K., Ash, P. E., Caulfield, T., Daughrity, L., Dunmore, J. H., Castanedes-Casey, M., Chew, J., et al. (2013). Antisense transcripts of the expanded C9ORF72 hexanucleotide repeat form nuclear RNA foci and undergo repeat-associated non-ATG translation in c9FTD/ALS. Acta Neuropathol 126, 829-844.
Gendron, T. F., Chew, J., Stankowski, J. N., Hayes, L. R., Zhang, Y. J., Prudencio, M., Carlomagno, Y., Daughrity, L. M., Jansen-West, K., Perkerson, E. A., et al. (2017). Poly(GP) proteins are a useful pharmacodynamic marker for C9ORF72-associated amyotrophic lateral sclerosis. Sci Transl Med 9.
Gijselinck, I., Van Mossevelde, S., van der Zee, J., Sieben, A., Engelborghs, S., De Bleecker, J., Ivanoiu, A., Deryck, O., Edbauer, D., Zhang, M., et al. (2016). The C9orf72 repeat size correlates with onset age of disease, DNA methylation and transcriptional downregulation of the promoter. Molecular Psychiatry 21, 1112-1124.
Gomez-Deza, J., Lee, Y. B., Troakes, C., Nolan, M., Al-Sarraj, S., Gallo, J. M., and Shaw, C. E. (2015). Dipeptide repeat protein inclusions are rare in the spinal cord and almost absent from motor neurons in C9ORF72 mutant amyotrophic lateral sclerosis and are unlikely to cause their degeneration. Acta Neuropathol Commun 3, 38.
Green, K. M., Glineburg, M. R., Kearse, M. G., Flores, B. N., Linsalata, A. E., Fedak, S. J., Goldstrohm, A. C., Barmada, S. J., and Todd, P. K. (2017). RAN translation at C9orf72-associated repeat expansions is selectively enhanced by the integrated stress response. Nature Communications 8, 2005.
Haeusler, A. R., Donnelly, C. J., and Rothstein, J. D. (2016). The expanding biology of the C9orf72 nucleotide repeat expansion in neurodegenerative disease. Nat Rev Neurosci 17, 383-395.
Hanwell, M. D., Curtis, D. E., Lonie, D. C., Vandermeersch, T., Zurek, E., and Hutchison, G. R. (2012). Avogadro: an advanced semantic chemical editor, visualization, and analysis platform. Journal of Cheminformatics 4, 17.
Hao, Z., Liu, L., Tao, Z., Wang, R., Ren, H., Sun, H., Lin, Z., Zhang, Z., Mu, C., Zhou, J., et al. (2019). Motor dysfunction and neurodegeneration in a C9orf72 mouse line expressing poly-PR. Nature communications 10, 2906.
Hartmann, H., Hornburg, D., Czuppa, M., Bader, J., Michaelsen, M., Farny, D., Arzberger, T., Mann, M., Meissner, F., and Edbauer, D. (2018). Proteomics and C9orf72 neuropathology identify ribosomes as poly-GR/PR interactors driving toxicity. Life Sci Alliance 1, e201800070.
Hayes, L. R., Duan, L., Bowen, K., Kalab, P., and Rothstein, J. D. (2020). C9orf72 arginine-rich dipeptide repeat proteins disrupt karyopherin-mediated nuclear import. eLife 9, e51685.
Henras, A. K., Plisson-Chastang, C., O'Donohue, M. F., Chakraborty, A., and Gleizes, P. E. (2015). An overview of pre-ribosomal RNA processing in eukaryotes. Wiley Interdiscip Rev RNA 6, 225-242.
Humphrey, W., Dalke, A., and Schulten, K. (1996). VMD: Visual molecular dynamics. Journal of Molecular Graphics 14, 33-38.
Jafarinia, H., van der Giessen, E., and Onck, P. R. (2020). Phase Separation of Toxic Dipeptide Repeat Proteins Related to C9orf72 ALS/FTD. Biophysical Journal 119, 843-851.
Jazurek, M., Ciesiolka, A., Starega-Roslan, J., Bilinska, K., and Krzyzosiak, W. J. (2016). Identifying proteins that bind to specific RNAs—focus on simple repeat expansion diseases. Nucleic acids research 44, 9050-9070.
Jorgensen, W. L., Chandrasekhar, J., Madura, J. D., Impey, R. W., and Klein, M. L. (1983). Comparison of simple potential functions for simulating liquid water. The Journal of Chemical Physics 79, 926-935.
Jovicic, A., Mertens, J., Boeynaems, S., Bogaert, E., Chai, N., Yamada, S. B., Paul, J. W., 3rd, Sun, S., Herdy, J. R., Bieri, G., et al. (2015). Modifiers of C9orf72 dipeptide repeat toxicity connect nucleocytoplasmic transport defects to FTD/ALS. Nat Neurosci 18, 1226-1229.
Kanekura, K., Harada, Y., Fujimoto, M., Yagi, T., Hayamizu, Y., Nagaoka, K., and Kuroda, M. (2018). Characterization of membrane penetration and cytotoxicity of C9orf72-encoding arginine-rich dipeptides. Sci Rep 8, 12740.
Kanekura, K., Yagi, T., Cammack, A. J., Mahadevan, J., Kuroda, M., Harms, M. B., Miller, T. M., and Urano, F. (2016). Poly-dipeptides encoded by the C9ORF72 repeats block global protein translation. Hum Mol Genet 25, 1803-1813.
Kognole, A. A., and MacKerell, A. D. (2020). Mg2+ Impacts the Twister Ribozyme through Push-Pull Stabilization of Nonsequential Phosphate Pairs. Biophysical Journal 118, 1424-1437.
Kwon, I., Xiang, S., Kato, M., Wu, L., Theodoropoulos, P., Wang, T., Kim, J., Yun, J., Xie, Y., and McKnight, S. L. (2014). Poly-dipeptides encoded by the C9orf72 repeats bind nucleoli, impede RNA biogenesis, and kill cells. Science 345, 1139-1145.
Lander, E. S., Linton, L. M., Birren, B., Nusbaum, C., Zody, M. C., Baldwin, J., Devon, K., Dewar, K., Doyle, M., FitzHugh, W., et al. (2001). Initial sequencing and analysis of the human genome. Nature 409, 860-921.
Lee, K.-H., Zhang, P., Kim, H. J., Mitrea, D. M., Sarkar, M., Freibaum, B. D., Cika, J., Coughlin, M., Messing, J., Molliex, A., et al. (2016). C9orf72 Dipeptide Repeats Impair the Assembly, Dynamics, and Function of Membrane-Less Organelles. Cell 167, 774-788.e717.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Liao, Y., Smyth, G. K., and Shi, W. (2014). featureCounts: an efficient general purpose program for assigning sequence reads to genomic features. Bioinformatics 30, 923-930.
Lin, Y., Mori, E., Kato, M., Xiang, S., Wu, L., Kwon, I., and McKnight, S. L. (2016). Toxic PR Poly-Dipeptides Encoded by the C9orf72 Repeat Expansion Target LC Domain Polymers. Cell 167, 789-802.e712.
Lopez-Gonzalez, R., Lu, Y., Gendron, T. F., Karydas, A., Tran, H., Yang, D., Petrucelli, L., Miller, B. L., Almeida, S., and Gao, F. B. (2016). Poly(GR) in C9ORF72-Related ALS/FTD Compromises Mitochondrial Function and Increases Oxidative Stress and DNA Damage in iPSC-Derived Motor Neurons. Neuron 92, 383-391.
Loveland, A. B., Svidritskiy, E., Susorov, D., Lee, S., Park, A., Demo, G., Gao, F.-B., and Korostelev, A. A. (2020). Ribosome inhibition by &lt;em&gt;C9ORF72&lt;/em&gt;-ALS/FTD-associated poly-PR and poly-GR proteins revealed by cryo-EM. bioRxiv, 2020.2008.2030.274597.
Mackenzie, I. R., Frick, P., Grasser, F. A., Gendron, T. F., Petrucelli, L., Cashman, N. R., Edbauer, D., Kremmer, E., Prudlo, J., Troost, D., et al. (2015). Quantitative analysis and clinico-pathological correlations of different dipeptide repeat protein pathologies in C9ORF72 mutation carriers. Acta Neuropathol 130, 845-861.
MacKerell, A. D., Bashford, D., Bellott, M., Dunbrack, R. L., Evanseck, J. D., Field, M. J., Fischer, S., Gao, J., Guo, H., Ha, S., et al. (1998). All-Atom Empirical Potential for Molecular Modeling and Dynamics Studies of Proteins. The Journal of Physical Chemistry B 102, 3586-3616.
MacKerell, A. D., Jr., Banavali, N., and Foloppe, N. (2000). Development and current status of the CHARMM force field for nucleic acids. Biopolymers 56, 257-265.
Mann, J. R., Gleixner, A. M., Mauna, J. C., Gomes, E., DeChellis-Marks, M. R., Needham, P. G., Copley, K. E., Hurtle, B., Portz, B., Pyles, N. J., et al. (2019). RNA Binding Antagonizes Neurotoxic Phase Transitions of TDP-43. Neuron 102, 321-338 e328.
Mansour, F. H., and Pestov, D. G. (2013). Separation of long RNA by agarose-formaldehyde gel electrophoresis. Anal Biochem 441, 18-20.
Marrink, S. J., Risselada, H. J., Yefimov, S., Tieleman, D. P., and de Vries, A. H. (2007). The MARTINI force field: coarse grained model for biomolecular simulations. J Phys Chem B 111, 7812-7824.
McEachin, Z. T., Gendron, T. F., Raj, N., Garcia-Murias, M., Banerjee, A., Purcell, R. H., Ward, P. J., Todd, T. W., Merritt-Garza, M. E., Jansen-West, K., et al. (2020). Chimeric Peptide Species Contribute to Divergent Dipeptide Repeat Pathology in c9ALS/FTD and SCA36. Neuron 107, 292-305.e296.
Mizielinska, S., Gronke, S., Niccoli, T., Ridler, C. E., Clayton, E. L., Devoy, A., Moens, T., Norona, F. E., Woollacott, I. O., Pietrzyk, J., et al. (2014a). C9orf72 repeat expansions cause neurodegeneration in Drosophila through arginine-rich proteins. Science 345, 1192-1194.
Mizielinska, S., Gronke, S., Niccoli, T., Ridler, C. E., Clayton, E. L., Devoy, A., Moens, T., Norona, F. E., Woollacott, I. O. C., Pietrzyk, J., et al. (2014b). C9orf72 repeat expansions cause neurodegeneration in Drosophila through arginine-rich proteins. Science 345, 1192-1194.
Moens, T. G., Niccoli, T., Wilson, K. M., Atilano, M. L., Birsa, N., Gittings, L. M., Holbling, B. V., Dyson, M. C., Thoeng, A., Neeves, J., et al. (2019). C9orf72 arginine-rich dipeptide proteins interact with ribosomal proteins in vivo to induce a toxic translational arrest that is rescued by eIF1A. Acta Neuropathol 137, 487-500.
Monticelli, L., Kandasamy, S. K., Periole, X., Larson, R. G., Tieleman, D. P., and Marrink, S.-J. (2008). The MARTINI Coarse-Grained Force Field: Extension to Proteins. Journal of Chemical Theory and Computation 4, 819-834.
Mori, K., Arzberger, T., Grasser, F. A., Gijselinck, I., May, S., Rentzsch, K., Weng, S. M., Schludi, M. H., van der Zee, J., Cruts, M., et al. (2013). Bidirectional transcripts of the expanded C9orf72 hexanucleotide repeat are translated into aggregating dipeptide repeat proteins. Acta Neuropathol 126, 881-893.
Nguyen, L., Montrasio, F., Pattamatta, A., Tusi, S. K., Bardhi, O., Meyer, K. D., Hayes, L., Nakamura, K., Banez-Coronel, M., Coyne, A., et al. (2020). Antibody Therapy Targeting RAN Proteins Rescues C9 ALS/FTD Phenotypes in C9orf72 Mouse Model. Neuron 105, 645-662.e611.
Odeh, H. M., and Shorter, J. (2020). Arginine-rich dipeptide-repeat proteins as phase disruptors in C9-ALS/FTD. Emerg Top Life Sci 4, 293-305.
Parrinello, M., and Rahman, A. (1981). Polymorphic transitions in single crystals: A new molecular dynamics method. Journal of Applied Physics 52, 7182-7190.
Potapova, T. A., and Gerton, J. L. (2019). Ribosomal DNA and the nucleolus in the context of genome organization. Chromosome Res 27, 109-127.
Quinlan, A. R., and Hall, I. M. (2010). BEDTools: a flexible suite of utilities for comparing genomic features. Bioinformatics 26, 841-842.
Radwan, M., Ang, C. S., Ormsby, A. R., Cox, D., Daly, J. C., Reid, G. E., and Hatters, D. M. (2020). Arginine in C9ORF72 Dipolypeptides Mediates Promiscuous Proteome Binding and Multiple Modes of Toxicity. Mol Cell Proteomics 19, 640-654.
Renton, A. E., Majounie, E., Waite, A., Simon-Sanchez, J., Rollinson, S., Gibbs, J. R., Schymick, J. C., Laaksovirta, H., van Swieten, J. C., Myllykangas, L., et al. (2011). A hexanucleotide repeat expansion in C9ORF72 is the cause of chromosome 9p21-linked ALS-FTD. Neuron 72, 257-268.
Saberi, S., Stauffer, J. E., Jiang, J., Garcia, S. D., Taylor, A. E., Schulte, D., Ohkubo, T., Schloffman, C. L., Maldonado, M., Baughn, M., et al. (2018). Sense-encoded poly-GR dipeptide repeat proteins correlate to neurodegeneration and uniquely co-localize with TDP-43 in dendrites of repeat-expanded C9orf72 amyotrophic lateral sclerosis. Acta Neuropathol 135, 459-474.
Schmidt, E. K., Clavarino, G., Ceppi, M., and Pierre, P. (2009). SUnSET, a nonradioactive method to monitor protein synthesis. Nature Methods 6, 275.
Shao, S., Murray, J., Brown, A., Taunton, J., Ramakrishnan, V., and Hegde, R. S. (2016). Decoding Mammalian Ribosome-mRNA States by Translational GTPase Complexes. Cell 167, 1229-1240 e1215.
Shao, Z., Zhang, Y., Yuan, G. C., Orkin, S. H., and Waxman, D. J. (2012). MAnorm: a robust model for quantitative comparison of ChIP-Seq data sets. Genome Biol 13, R16.
Shi, K. Y., Mori, E., Nizami, Z. F., Lin, Y., Kato, M., Xiang, S., Wu, L. C., Ding, M., Yu, Y., Gall, J. G., et al. (2017). Toxic PR<sub>n</sub> poly-dipeptides encoded by the <em>C9orf72</em>repeat expansion block nuclear import and export. Proceedings of the National Academy of Sciences 114, E1111-E1117.
Shi, Y., Lin, S., Staats, K. A., Li, Y., Chang, W. H., Hung, S. T., Hendricks, E., Linares, G. R., Wang, Y., Son, E. Y., et al. (2018). Haploinsufficiency leads to neurodegeneration in C9ORF72 ALS/FTD human induced motor neurons. Nature medicine 24, 313-325. 10.1038/nm.4490.
Smith, T., Heger, A., and Sudbery, I. (2017). UMI-tools: modeling sequencing errors in Unique Molecular Identifiers to improve quantification accuracy. Genome Res 27, 491-499.
Sonobe, Y., Ghadge, G., Masaki, K., Sendoel, A., Fuchs, E., and Roos, R. P. (2018). Translation of dipeptide repeat proteins from the C9ORF72 expanded repeat is associated with cellular stress. Neurobiol Dis 116, 155-165.
Suh, E., Lee, E. B., Neal, D., Wood, E. M., Toledo, J. B., Rennert, L., Irwin, D. J., McMillan, C. T., Krock, B., Elman, L. B., et al. (2015). Semi-automated quantification of C9orf72 expansion size reveals inverse correlation between hexanucleotide repeat number and disease duration in frontotemporal degeneration. Acta Neuropathol 130, 363-372.
Suomivuori, C.-M., Latorraca, N. R., Wingler, L. M., Eismann, S., King, M. C., Kleinhenz, A. L. W., Skiba, M. A., Staus, D. P., Kruse, A. C., Lefkowitz, R. J., et al. (2020). Molecular mechanism of biased signaling in a prototypical G protein-coupled receptor. Science 367, 881-887.
Tabet, R., Schaeffer, L., Freyermuth, F., Jambeau, M., Workman, M., Lee, C. Z., Lin, C. C., Jiang, J., Jansen-West, K., Abou-Hamdan, H., et al. (2018). CUG initiation and frameshifting enable production of dipeptide repeat proteins from ALS/FTD C9ORF72 transcripts. Nat Commun 9, 152.
Tao, Z., Wang, H., Xia, Q., Li, K., Li, K., Jiang, X., Xu, G., Wang, G., and Ying, Z. (2015). Nucleolar stress and impaired stress granule formation contribute to C9orf72 RAN translation-induced cytotoxicity. Hum Mol Genet 24, 2426-2441.
Uversky, V. N. (2013). Disorder in the lifetime of a protein. Intrinsically Disord Proteins 1, e26782-e26782.
van Blitterswijk, M., DeJesus-Hernandez, M., Niemantsverdriet, E., Murray, M. E., Heckman, M. G., Diehl, N. N., Brown, P. H., Baker, M. C., Finch, N. A., Bauer, P. O., et al. (2013). Association between repeat sizes and clinical and pathological characteristics in carriers of C9ORF72 repeat expansions (Xpansize-72): a cross-sectional cohort study. Lancet Neurol 12, 978-988.
Vanneste, J., Vercruysse, T., Boeynaems, S., Sicart, A., Van Damme, P., Daelemans, D., and Van Den Bosch, L. (2019). C9orf72-generated poly-GR and poly-PR do not directly interfere with nucleocytoplasmic transport. Scientific Reports 9, 15728.
Vanommeslaeghe, K., Hatcher, E., Acharya, C., Kundu, S., Zhong, S., Shim, J., Darian, E., Guvench, O., Lopes, P., Vorobyov, I., et al. (2010). CHARMM general force field: A force field for drug-like molecules compatible with the CHARMM all-atom additive biological force fields. Journal of Computational Chemistry 31, 671-690.
Verlet, L. (1967). Computer “Experiments” on Classical Fluids. I. Thermodynamical Properties of Lennard-Jones Molecules. Physical Review 159, 98-103.
Wen, X., Tan, W., Westergard, T., Krishnamurthy, K., Markandaiah, S. S., Shi, Y., Lin, S., Shneider, N. A., Monaghan, J., Pandey, U. B., et al. (2014). Antisense proline-arginine RAN dipeptides linked to C9ORF72-ALS/FTD form toxic nuclear aggregates that initiate in vitro and in vivo neuronal death. Neuron 84, 1213-1225.
Westergard, T., McAvoy, K., Russell, K., Wen, X., Pang, Y., Morris, B., Pasinelli, P., Trotti, D., and Haeusler, A. (2019). Repeat-associated non-AUG translation in C9orf72-ALS/FTD is driven by neuronal excitation and stress. EMBO Mol Med 11.
White, M. R., Mitrea, D. M., Zhang, P., Stanley, C. B., Cassidy, D. E., Nourse, A., Phillips, A. H., Tolbert, M., Taylor, J. P., and Kriwacki, R. W. (2019). C9orf72 Poly(PR) Dipeptide Repeats Disturb Biomolecular Phase Separation and Disrupt Nucleolar Function. Mol Cell 74, 713-728 e716.
Wingett, S. W., and Andrews, S. (2018). FastQ Screen: A tool for multi-genome mapping and quality control. F1000Res 7, 1338.
Yamada, S. B., Gendron, T. F., Niccoli, T., Genuth, N. R., Grosely, R., Shi, Y., Glaria, I., Kramer, N. J., Nakayama, L., Fang, S., et al. (2019). RPS25 is required for efficient RAN translation of C9orf72 and other neurodegenerative disease-associated nucleotide repeats. Nature Neuroscience 22, 1383-1388.
Yin, S., Lopez-Gonzalez, R., Kunz, R. C., Gangopadhyay, J., Borufka, C., Gygi, S. P., Gao, F. B., and Reed, R. (2017). Evidence that C9ORF72 Dipeptide Repeat Proteins Associate with U2 snRNP to Cause Mis-splicing in ALS/FTD Patients. Cell Rep 19, 2244-2256.
Zhang, K., Daigle, J. G., Cunningham, K. M., Coyne, A. N., Ruan, K., Grima, J. C., Bowen, K. E., Wadhwa, H., Yang, P., Rigo, F., et al. (2018a). Stress Granule Assembly Disrupts Nucleocytoplasmic Transport. Cell 173, 958-971.e917.
Zhang, Y. J., Gendron, T. F., Ebbert, M. T. W., O'Raw, A. D., Yue, M., Jansen-West, K., Zhang, X., Prudencio, M., Chew, J., Cook, C. N., et al. (2018b). Poly(GR) impairs protein translation and stress granule dynamics in C9orf72-associated frontotemporal dementia and amyotrophic lateral sclerosis. Nat Med 24, 1136-1142.
Zhang, Y. J., Guo, L., Gonzales, P. K., Gendron, T. F., Wu, Y., Jansen-West, K., O'Raw, A. D., Pickles, S. R., Prudencio, M., Carlomagno, Y., et al. (2019). Heterochromatin anomalies and double-stranded RNA accumulation underlie C9orf72 poly(PR) toxicity. Science (New York, NY) 363.
Zhou, Q., Lehmer, C., Michaelsen, M., Mori, K., Alterauge, D., Baumjohann, D., Schludi, M. H., Greiling, J., Farny, D., Flatley, A., et al. (2017). Antibodies inhibit transmission and aggregation of C9orf72 poly-GA dipeptide repeat proteins. EMBO Molecular Medicine 9, 687-702.
Zhu, Q., Jiang, J., Gendron, T. F., McAlonis-Downes, M., Jiang, L., Taylor, A., Diaz Garcia, S., Ghosh Dastidar, S., Rodriguez, M. J., King, P., et al. (2020). Reduced C9ORF72 function exacerbates gain of toxicity from ALS/FTD-causing repeat expansion in C9orf72. Nat Neurosci 23, 615-624.
Zhu, X., Lopes, P. E. M., and MacKerell Jr, A. D. (2012). Recent developments and applications of the CHARMM force fields. WIREs Computational Molecular Science 2, 167-185.
Zilller, M. J., Ortega, J. A., Quinlan, K. A., Santos, D. P., Gu, H., Martin, E. J., Galonska, C., Pop, R., Maidl, S., Di Pardo, A., et al. (2018). Dissecting the Functional Consequences of De Novo DNA Methylation Dynamics in Human Motor Neuron Differentiation and Physiology. Cell stem cell 22, 559-574.e559. 10.1016/j.stem.2018.02.012.

1. A single-stranded RNA (ssRNA) construct comprising a sequence that is identical to the sequence of a fragment of a ribosomal RNA (rRNA) subunit selected from the group consisting of the 28S, 18S, and 5.8S subunits, or a fragment of the 5′ETS or ITS1 region of the 47S rRNA precursor, wherein the ssRNA construct comprises one or more modified nucleotides.

2. The ssRNA construct of claim 1, wherein the ssRNA construct comprises a sequence that is identical to the sequence of a fragment of the rRNA 28S subunit.

3. The ssRNA construct of claim 1, wherein the fragment of the rRNA 28S subunit comprises all or part of the sequence of SEQ ID NO: 1.

4. The ssRNA construct of claim 1, wherein the fragment of the rRNA 28S subunit includes nucleotides 2740 and 2820 of SEQ ID NO: 3.

5. The ssRNA construct of claim 1, wherein the ssRNA construct comprises a sequence that is identical to the sequence of a fragment of the rRNA 5.8S subunit.

6. The ssRNA construct of claim 5, wherein the fragment of the rRNA 5.8S subunit comprises all or part of the sequence SEQ ID NO: 2.

7. The ssRNA construct of claim 1, wherein the fragment consists of up to 30 nucleotides.

8. The ssRNA construct of claim 1, wherein the modified nucleotides comprise one or more 2′-modified ribose sugars.

9. The ssRNA construct of claim 1, wherein the modified nucleotides are 2′-O-alkyl nucleotides, 2′-deoxy-2′-fluoro nucleotides, 2′-deoxy nucleotides, 2′-H (deoxyribonucleotides), or a combination thereof.

10. The ssRNA construct of claim 1, wherein the modified nucleotides are 2′-O-methyl nucleotides.

11. The ssRNA construct of claim 1, wherein the modified nucleotides are at the 5′ end and/or the 3′ end.

12. The ssRNA construct of claim 1 comprising a structure of mGmGmUmCmUrCrCrArArGrGrUrGrArAmCmAmGmCmC (SEQ ID NO: 8).

13. (canceled)

14. (canceled)

15. A method for treating or preventing amyotrophic lateral sclerosis or frontotemporal dementia in a subject in need thereof, comprising administering to the subject a therapeutically effective amount of an ssRNA construct that inhibits dipeptide repeat (DPR) protein-ribosomal RNA (rRNA) interaction, wherein the ssRNA construct comprises a sequence that is identical to the sequence of a fragment of a ribosomal RNA (rRNA) or a rRNA precursor.

16. The method of claim 15, wherein the subject harbors a heterozygous intronic hexanucleotide (GGGGCC)ₙ repeat expansion in C9ORF72 (C9-HRE) gene.

17. The method of claim 15, wherein the subject has an increased expression of one or more of poly-glycine-proline (polyGP), poly-glycine-alanine (polyGA), poly-glycine-arginine (polyGR), poly-proline-arginine (polyPR), and poly-proline-alanine (polyPA) as compared to that observed in a healthy subject.

18. The method of claim 15, wherein the subject has an increased expression of poly-GR and/or poly-PR.

19. The method of claim 15, wherein the subject exhibits one or more signs of impaired ribosomal homeostasis and function selected from the group consisting of a reduced total rRNA level, an increased nucleocytoplasmic (N/C) ratio of rRNA, or a decreased level of de novo protein translation as compared to that observed in a healthy subject.

20. The method of claim 15, wherein the ssRNA comprises one or more modified nucleotides.

21. The method of claim 15, wherein the modified nucleotides comprise 2′-modified ribose sugars.

22. The method of claim 15, wherein the modified nucleotides are 2′-O-alkyl nucleotides, 2′-deoxy-2′-fluoro nucleotides, 2′-deoxy nucleotides, 2′-H (deoxyribonucleotides), or a combination thereof.

23. (canceled)
